SEQUENCE LISTING (3 sequences)

SEQ ID NO: 1 - protein, 92 aa, artificial
  bis(azide) di-functionalized polypeptide
  MOD_RES (1)..(1): Xaa is Gly modified with azide
  MOD_RES (92)..(92): Xaa is Gly modified with azide
XGKEKEKEKEKEKEKEKEKEKEKEKEKEKEKEKEKEKEKEKEGPQGIWGQKEKEKEKEKEKEKEKEKEKEKEKEKEKEKEKEKEKEKEKEGX

SEQ ID NO: 2 - protein, 47 aa, artificial
  synthetic peptide poly(KE)1 of disclosure
CGGKEKEKEKEKEKEKEKEKEKEKEKEKEKEKEKEKEKEKEKEGPQG

SEQ ID NO: 3 - protein, 47 aa, artificial
  synthetic peptide poly(KE)2 of disclosure
CGGKEKEKEKEKEKEKEKEKEKEKEKEKEKEKEKEKEKEKEKEIWGQ

(19) United States
(12) Patent Application Publication
(10) Pub. No.: US 2020/0009194 A1
(43) Pub. Date: Jan. 9, 2020

(54) SYSTEMS AND METHODS FOR HEMATOPOIETIC CELL EXPANSION UTILIZING HYDROGELS

(71) Applicant: Fred Hutchinson Cancer Research Center, Seattle, WA (US)

(72) Inventor: Colleen Delaney, Seattle, WA (US)

(73) Assignee: Fred Hutchinson Cancer Research Center, Seattle, WA (US)

(21) Appl. No.: 16/480,481

(22) PCT Filed: Jan. 24, 2018
(86) PCT No.: PCT/US18/15101

§ 371 Date: Jul. 24, 2019

Related U.S. Application Data
(60) Provisional application No. 62/449,998, filed on Jan. 24, 2017.

(51) Int. Cl. A61K 35/28; C12N 5/0789

(52) U.S. Cl. CPC A61K 35/28; C12N 5/0647; C12N 2501/125; C12N 2501/26; C12N 2510/00; C12N 2501/2306; C12N 2501/2303; C12N 2533/30; C12N 2533/50; C12N 2501/145

(57) ABSTRACT

Systems and methods to expand hematopoietic stem cell (HSC) using zwitterionic hydrogels (ZTG) are described. Expansion using the disclosed systems and methods results in HSC populations with (i) an increased proportion of HSC versus partially or fully differentiated cells, (ii) proportionally lower cell surface expression levels of differentiation/maturation markers, (iii) reduced metabolic rates following expansion, and/or (iv) a greater proportion of quiescent cells following expansion, as compared to currently available clinical expansion methods.

### CROSS REFERENCE TO RELATED APPLICATION

This application claims priority to U.S. Provisional Patent Application No. 62/449,998 filed on Jan. 24, 2017, which is incorporated herein by reference in its entirety as if fully set forth herein.

### FIELD OF THE DISCLOSURE

The current disclosure provides ex vivo systems and methods to expand CD34+ hematopoietic cells using ultra-low fouling hydrogels, such as zwitterionic hydrogels (ZTG). CD34+ hematopoietic cell populations cultured using these systems and methods have, following expansion, an increased proportion of hematopoietic stem cells (HSC) versus partially or fully differentiated cells, proportionally lower cell surface expression levels of differentiation/maturation markers, reduced metabolic rates, and/or a greater proportion of quiescent cells, as compared to currently available clinical expansion methods.

### BACKGROUND OF THE DISCLOSURE

Hematopoietic cell transplantation (HCT) is an effective and widely used therapy with curative intent for a number of hematologic malignancies and non-malignant disorders. A total of 88,063 hematopoietic cell transplants were performed in the USA between 2009 and 2013, with 37,768 of these being from allogeneic related and unrelated stem cell donors (Center for International Blood and Marrow Transplant Research, Transplant Activity Report Covering 2009-2013). However, the majority of patients in need of an allogeneic HCT will not have the preferred donor, namely an HLA-matched related donor.

In addition, only 50% of Caucasian patients and far fewer patients of mixed race/minority backgrounds will be able to identify an unrelated, suitably matched and available adult donor (Gragert et al., N Engl J Med, 371(4):339 (2014)). For these patients, umbilical cord blood (CB) is now commonly used as a source of hematopoietic stem and progenitor cells (HSPC) for allogeneic HCT. Cord blood has the distinct advantage of less stringent HLA-matching requirements between donor and host, allowing nearly all patients to identify cord blood donors without an increased risk of graft-versus-host disease, even in the setting of mismatched donors. Cord blood transplant (CBT) recipients have also been shown to have lower disease relapse rates post-transplant compared to those receiving conventional unrelated donor bone marrow (BM) or peripheral blood stem cell transplants. Furthermore, a recent study evaluated the impact of pre-transplant minimal residual disease in patients undergoing a first allogeneic stem cell transplant. This study demonstrated that cord blood transplant recipients had a survival advantage compared to matched and mismatched unrelated donor transplant recipients (Milano et al., N Engl J Med, 375(10):944 (2016)). Despite these advantages, the low HSPC dose available in a single- or double-unit CBT significantly delays hematopoietic recovery and results in a higher risk of graft failure and early transplant-related mortality, limiting the more widespread use of this stem cell source (Wagner et al., Blood, 100:1611 (2002); Ballen, Blood, 122:491 (2013); Smith & Wagner, Br J Haematol, 147:246 (2009)).

To increase the absolute numbers of HSPC available for clinical applications in the setting of HCT, researchers have long sought to identify culture conditions suitable for ex vivo expansion of HSPC populations (Dahlberg, Blood, 117:6083 (2011)). Early cytokine-mediated expansion strategies promote HSPC proliferation, but notably also trigger cell differentiation, diminishing their therapeutic value (Murray et al., Exp Hematol, 27:1019 (1999), Ratajczak, Curr Opin Hematol, 15:293 (2008)).

More recent efforts include targeted ex vivo manipulation of molecular pathways known to play a role in hematopoietic stem cell (HSC) fate (Pineault, Exp Hematol, 43:498 (2015)). For example, HSPC populations exposed to an optimized balance of stimulatory and inhibitory factors in a fed-batch reactor have exhibited promising expansion rates and repopulation properties (Csaszar et al., Cell Stem Cell, 10:218 (2012)). The use of an engineered Notch ligand (Delta1) to activate endogenous Notch signaling in CB-derived HSPC, thereby inhibiting ex vivo differentiation and resulting in a significant increase in the absolute number of CD34+ cells (e.g., HSPCs) available for clinical applications, has also been used. This approach has been evaluated in a number of clinical trials and shown to be safe and reduce the time to hematopoietic recovery in the myeloablative CBT setting (Delaney et al., Nat Med, 16:232 (2010), Delaney et al., Lancet Haematology, (2016)). Other more recent efforts include preparation of HSPC products using small molecules, such as products prepared by methods employing the aryl hydrocarbon antagonist SR1 and the pyrimidoindole derivatives (UM family of molecules) (Boitano et al., Science, 329:1345 (2010), Fares et al., Science, 345:1509 (2014)). These methods have also been shown to promote the expansion of CB-derived HSPC populations that possess robust repopulation activity (e.g., more rapid neutrophil and/or platelet count recovery). These notable advances have led to improvements in short-term hematopoietic reconstitution, such as a reduced time to neutrophil recovery in a number of CBT trials. However, these state-of-the-art culture strategies continue to result in significant HSC differentiation into progenitor cells. Pineault, Exp. Hematol, 43:498 (2015). While the currently available culture strategies that result in significant differentiation to progenitor cells provide benefits for short-term engraftment, this differentiation can negatively affect long-term reconstitution. Thus, culture strategies that further reduce HSC differentiation are needed for cell products intended to provide long-term hematopoietic reconstitution, for example, for correction of genetic diseases such as sickle cell and thalassemia.

### SUMMARY OF THE DISCLOSURE

Provided here are culture systems and methods to expand CD34+ hematopoietic stem progenitor cell (HSPC) populations using ultra-low fouling hydrogels, such as zwitterionic hydrogels (ZTG). Expansion using these systems and methods results in cell populations with an increased proportion of hematopoietic stem cells (HSC) versus partially or fully differentiated cells, proportionally lower cell surface expression levels of differentiation/maturation markers, reduced metabolic rates, and/or a greater proportion of quiescent cells, as compared to currently available clinical expansion methods. Each of these characteristics is beneficial for cell populations expanded for research or therapeutic uses requiring long-term hematopoietic reconstitution, such as those related to correction of genetic diseases (e.g., sickle cell and thalassemia and others described herein).

In particular embodiments, the hydrogel includes a ZTG. In particular embodiments, the ZTG includes a zwitterionic polymer. In particular embodiments, the zwitterionic polymer includes a four-arm poly(carboxybetaine acrylamide) tetracyclooctyne. In particular embodiments, the ZTG includes a zwitterionic polymer crosslinker that is degraded by a product released by the expanding cell population. In particular embodiments, the crosslinker is a peptide. In particular embodiments, the peptide includes a poly(EK) crosslinker. In particular embodiments, the poly(EK) crosslinker includes a bis(azide) di-functionalized polypeptide. In particular embodiments, the bis(azide) di-functionalized polypeptide includes Azide-GG-(KE)₂₀-GPQGIWGQ-(KE)₂₀GG-Azide (SEQ ID NO: 1).

### BRIEF DESCRIPTION OF ABBREVIATED EXPERIMENTAL CONDITIONS

ZTGₒₚₜ: a zwitterionic hydrogel (ZTG) hardness of 0.7 kPa; a seeding density of 1.2 million cells/ml; a medium including human stem cell factor (SCF), FMS-like tyrosine kinase 3 ligand (FLT3), thrombopoietin (TPO), interleukin-6 (IL-6), and interleukin-3 (IL-3); 14 days for a first passage and 10 days for a second passage; cells cultured within the three-dimensional (3D) ZTG environment.

ZTG₁₄: the same conditions as ZTGₒₚₜ, but the first 14 day culture only (no second passage).

ZTG_{2D}: an essentially flat ZTG without cell encapsulation. Cells for expansion are added on top of the culture well after the ZTG_{2D} has been formed. All references to ZTG refer to a 3D ZTG unless specifically denoted to be in 2D form.

### BRIEF DESCRIPTION OF THE FIGURES

FIGS. 1A-1E. Construction of biodegradable zwitterionic hydrogel (ZTG) allows the expansion of CD34+ cord blood (CB) cell populations. Chemical structures of the hydrogel components including: (FIG. 1A) a four-armed poly(carboxybetaine acrylamide) tetracyclooctyne and (FIG. 1B) a crosslinker with a metalloproteinase-cleavable motif. (FIG. 1C) a Huisgen cycloaddition used to form a 3D ideal network hydrogel through a step-growth polymerization mechanism. (FIG. 1D) Molecular description of prepared star-shaped pCBAA polymers. Summary of functionalized polymer weight detected by NMR or GPC and conversion rate of each step. (FIG. 1E) Synthesis of zwitterionic star-shaped polymer. N₃-terminated star-shaped pCBAA was produced by atom-transfer radical-polymerization and subsequent azide substitution. Then, the azide groups on pCBAA were converted into NH₂ groups using a ‘click’ reaction. Finally, DIFO₃ was functionalized to the end of pCBAA polymer via an EDC/NHS reaction.

FIG. 2. Zwitterionic polymer and peptide are ultra-low fouling in complex protein solutions. Total protein adsorption was measured on an surface plasmon resonance (SPR) sensor after injection of cell culture medium and fresh HSPC lysates on polystyrene, pCBAA and poly(KE)₁: CGG(KE)₂₀GPQG (SEQ ID NO: 2) and poly(KE)₂: CGG(KE)₂₀IWGQ (SEQ ID NO: 3) films.

FIGS. 3A-3D. ZTG culture prolongs the viability of HSPCs but cell division in ZTG requires growth factors (GFs). (FIG. 3A) Viability of and (FIG. 3B) Total nucleated cell (TNC) fold expansion of ZTG-encapsulated cord blood CD34⁺ HSPCs, cultured in SFEM II medium with (triangle) or without (circle) 5 growth factors, while cells cultured in control conditions with (square) or without (diamond) growth factors were set as controls. Mean±SEM is shown; n=3 independent experiments. (FIG. 3C) Fold expansion of CD34+ cells between 0 and 14 days of culturing in ZTG or bare flask conditions. ZTG encapsulated HSPCs were cultured in SFEM II medium and were analyzed (square data points show addition of five growth factors; circular data points show no additional growth factors). Bare flask cell culture with SFEM II medium were used as controls (triangle (point upwards) data points show addition of growth factors; inverted triangle (point downwards) data points show no additional growth factors). (FIG. 3D) Representative carboxyfluorescein succinimidyl ester (CFSE) cell labeling profiles of CD34+ CFSE populations in ZTG after 7 days with 0 growth factors (GF) and with 5 growth factors, as well as cultured in TOPS after 4 days with 5 growth factors.

FIGS. 4A, 4B. Examination of CD34+ HSPC population seeding density for ZTG culture. (FIG. 4A) Kinetics of TNC expansion at different seeding densities in ZTG culture (squares: 0.6×10⁶ cells/ml; circles: 1.2×10⁶ cells/ml; triangles: 2.5×10⁶ cells/ml). 1.2×10⁶ cells/ml was selected as the optimal seeding density. Mean±SEM is shown; n=3 independent experiments. (FIG. 4B) Immunofluorescence for ZTG₁₄ cells with 1.2×10⁶ cells/ml seeding density after staining with DAPI and anti-CD34 antibody.

FIGS. 5A, 5B. Effect of mechanical property on expanded cells from ZTG culture. (FIG. 5A) Dynamic total cell number increase in ZTG with different mechanical properties [ZTGₒₚₜ (0.7 kPa), ZTG_{medium} (1.9 kPa), ZTG_{high} (5 kPa); hydrogels with lower mechanical properties are too soft to handle]. (FIG. 5B) Percentage of CD34+ cells after each culture condition. ns indicates no significant difference.

FIG. 6. CD34 expression comparison between ZTG and other expansion systems. The % of CD45+ cells in the ZTG group is significantly higher than all other tested expansion systems.

FIGS. 7A, 7B. Dynamic cell cycle analysis. (FIG. 7A) Dynamic cell cycle analysis by FACS using anti-Ki-67 and Hoechst 33342 staining for cells in ZTGₒₚₜ culture. (FIG. 7B) Representative phase contrast image of ZTGₒₚₜ cell at day 24 in culture.

FIGS. 8A-8C. ZTGₒₚₜ cultured cells show delayed entry into cell cycle upon subsequent culture. Dynamic change of cell cycle subsets after transferring cells before (Fresh) and after ZTGₒₚₜ culture into control conditions. Percentage of cells in: (FIG. 8A) G₀; (FIG. 8B) G₁; and (FIG. 8C) 5-G₂M are shown. Mean±SEM is shown; n=3 independent experiments. *p<0.05, **p<0.001, ***p<0.0005 by 2-tail t-test.

FIG. 9. Schematic illustration of the ZTGₒₚₜ culture procedure and experimental outline: freshly isolated HSPCs were mixed with the click-reactive zwitterionic components to form cell-ZGT constructs, which were cultured in expansion media for 14 days (ZTG₁₄). A second expansion was performed by dividing the ZTG₁₄ population among new ZGT at an optimal seeding concentration (see FIG. 4A) and culturing them for another 10 days before final harvest for further experimentation. The resulting ZTGₒₚₜ cells were injected into NSG mice for primary and secondary transplantation.

FIGS. 10A-10D. Effect of additional passages on expanded cells from ZTG culture. (FIG. 10A) Dynamic total cell number increase in ZTG culture; (FIG. 10B) mean fluorescent intensity of (MFI) of CD34; (FIG. 100) MFI of CD45RA; (FIG. 10D) MFI of lineage (Lin) marker cocktail. NS indicates no significant difference. * indicates a significant difference (P<0.05). Values represent means±SD, n=3 independent experiments.

FIG. 11. Dynamic changes in cell counts and CD34 expression during ZTGₒₚₜ culture.

FIGS. 12A, 12B. Significant differentiation in HYSTEM® (Glycosan Biosystems, Inc., Salt Lake City, Utah) hydrogels lead to lower fold expansion of CD34+ cells after 24 days. (FIG. 12A) Percentage of CD34+ cells after culture in ZTG and HYSTEM® hydrogels for 14 days and 24 days. (FIG. 12B) Fold expansion of CD34+ cells after culture in ZTG and HYSTEM® hydrogels for 14 days and 24 days. * indicates a significant difference (P<0.05). Values represent mean±SD, n=3 independent experiments.

FIGS. 13A, 13B. (FIG. 13A) Fold increase in colony-forming units (CFUs) after first and second expansions in ZTG. (FIG. 13B) Mice were treated by injection with 200,000 cells harvested at the end of Day-14 or Day-24, which theoretically corresponded to the progeny of 10,000 and 667 Day-0 CD34+ cells, respectively. The level of long-term human engraftment (% hCD45) in mouse bone marrow 20 weeks after transplantation is shown. Horizontal lines indicate the average value for each group. * indicates a significant difference (P<0.05).

FIGS. 14A-14F. ZTG culture results in minimal differentiation of CD34⁺ CB cells during ex vivo expansion. (FIG. 14A) Photomicrograph of Wright-Giemsa-stained fresh HSPCs and cells after culture in different conditions, and average cell diameter. Scale bar: 30 um. (FIG. 14B) The distribution of and (FIG. 14C) FACS profile of fresh, control-, DXIₒₚₜ-, and ZTGₒₚₜ-cultured cells according to CD34 and lineage (CD7, CD14, CD15, CD19 and CD56) marker expression determined by flow cytometry. (FIG. 14D) ZTGₒₚₜ-culture results in cells that predominantly express cell surface markers consistent with phenotypically primitive HSC. Representative FACS profiles of the most primitive HSC phenotype subset (CD34⁺CD38⁻CD45RA⁻CD90⁺CD49f⁺) from fresh, control, DXIₒₚₜ and ZTGₒₚₜ conditions. Consecutive gating was applied from top to down. (FIG. 14E) Representative FACS profiles of CD34⁺CD45RA⁻ populations in fresh, control-, DXIₒₚₜ, and ZTGₒₚₜ-cultured cells. (FIG. 14F) Three graphs depicting cell fold expansion (CFUs) after culture in ZTG, DXI, and TOPS: (left panel) Granulocyte, erythrocyte, monocyte, megakaryocyte (GEMM), (middle panel) granulocyte, monocyte (GM), and (right panel) erythroid burst-forming units (BFU-E). In addition, as shown in FIG. 20, colony-forming unit (CFU) assays revealed that GEMM, GM and BFU-E were all enhanced when cells were cultured within ZTG. These data all suggest that cells expanded within ZTG are more primitive HSC, different than those HSPC expanded in Delta1^{ext-IgG} or TOPS systems.

FIG. 15. Fold expansion of phenotypically defined hematopoietic cell subsets after culture in each condition.

FIGS. 16A-16D. ZTG cultured cells retain their capacity for subsequent expansion. (FIG. 16A) Schematic representation of the experimental design. Cord blood CD34⁺ HSPCs were expanded in the ZTGₒₚₜ condition, after which cells were harvested and further expanded in DXIₒₚₜ and control conditions. These secondary groups of expanded cells were harvested and injected into NSG mice, with progeny doses based on a founding CD34⁺ population of 1000 for each group. (FIG. 16B) Flow cytometry analysis of subsequently expanded CD34⁺ populations. (FIG. 16C) Fold change of the subsequent expansion in DXIₒₚₜ or control conditions. (FIG. 16D) Human engraftment in NSG BM after indicated time points.

FIGS. 17A-17F. Culture of CD34⁺ CB cells in the ZTGₒₚₜ condition promotes expansion of long-term repopulating HSC (LT-HSCs). (FIG. 17A) Percent human cells in the bone marrow, with fresh cells shown with squares and ZTGₒₚₜ cells shown with circles. Mice were injected with either fresh cord blood CD34⁺ HSPCs or ZTGₒₚₜ-expanded progeny at doses normalized to their founding HSPC population. The percentage of human CD45⁺ cells in the mouse bone marrow at week 24-30 is shown. Horizontal lines indicate the average value for each group. (FIGS. 17B and 17C). Limiting dilution analysis of NSG engraftment. (FIG. 17B) Summary of primary NSG engraftment data from different time points. Cells from different expansion conditions were injected into NSG mice at different doses. Human engraftment was examined at different time intervals. Human engraftment higher than 0.1% was considered a positive response. (FIG. 17C) Poisson statistics were applied to the data in FIG. 17B and severe combined immune deficient (SCID) repopulating cell (SRC) were calculated and presented in different scenarios. Progeny: CD34⁺ starting cells (day 0 equivalent). Cell dose: Actual total cells infused. (FIG. 17D) In vivo data analyzed at indicated time post-transplantation presented as heat-map. (FIG. 17E) The average percentage of human engraftment in bone marrow from mice groups injected with various numbers of starting CD34⁺ and CD34+ cells cultured in ZTG at early (4 weeks), median (12-14 weeks), and late (24-30 weeks) time points. (FIG. 17F) SRC frequency from mice groups injected with fresh CD34⁺ and CD34+ cells cultured in ZTG, DXI, and TOPS at early (4 weeks), median (12-14 weeks), and late (24-30 weeks) time points. Error bars are shown and represent a 95% confidence interval.

FIGS. 18A, 18B. (FIG. 18A) Linear regression analysis of data from FIG. 17A. Solid lines indicate the best-fit linear regression model for each data set. Dotted lines represent 95% confidence interval. (FIG. 18B) LT-HSC numbers before and after culture in the ZTGₒₚₜ condition.

FIGS. 19A, 19B. (FIG. 19A) Levels of human engraftment in NSG mice transplanted with different cell doses. (FIG. 19B) ZTGₒₚₜ culture does not affect the lineage repopulating ability. Representative flow cytometry dot plots from BM samples flushed from transplanted mice at week 24. Pooled BM samples were analyzed (top: Pooled BM from 4 mice receiving 10,000 fresh HSPCs; bottom: Pooled BM from 5 mice receiving ZTGₒₚₜ cells expanded from 100 fresh HSPCs). Progeny: CD34⁺ starting cells (day 0 equivalent).

FIG. 20. Summary of secondary NSG engraftment plan and data.

FIGS. 21A-21D. ZTG cultures were initiated from bone marrow (BM) CD34+ HSPC (rather than CB CD34+ HSPC, as in the previous FIGs.). ZTG cultures initiated from BM CD34+ HSPC result in minimal generation of BM HSPCs during ex vivo expansion. Representative flow cytometry dot plot for (FIG. 21A) fresh and (FIG. 21B) ZTGₒₚₜ cells. FSC: forward scatter; SSC: side scatter. (FIG. 21C) Fold expansion of total and CD34+ cells after ZTGₒₚₜ culture. (FIG. 21D) CFU numbers per 1000 Fresh or ZTGₒₚₜ cells.

FIG. 22. In vivo function of expanded BM-CD34⁺ HSPCs from ZTGₒₚₜ culture. Levels of human engraftment and lineage repopulating in NSG mice transplanted with different cell doses at week 24.

FIGS. 23A-23C. ZTG culture avoids excessive ROS production. (FIG. 23A) Hydrophobicity-induced nonspecific cell-matrix/substrate interaction leads to excessive ROS production. These generated ROS can nonspecifically activate and deactivate intracellular pathways and result in defective self-renewal of HSCs. (FIG. 23B) Intracellular ROS was measured with DCFH2-DA. (FIG. 23C) Mitochondrial superoxide was measured with MITOSOX® (Life Technologies Corp., Carlsbad, Calif.) Red after 1-day culture.

FIG. 24. ZTG culture avoids excessive cellular ROS production. Representative FACS profiles of cellular ROS level from cells after 24-hour culture in fresh, ZTG, HYSTEM®_{3D}, control, DXI, ZTG_{2D} and HYSTEM®_{2D} conditions.

FIG. 25. ZTG culture avoids excessive cellular mitochondrial O₂⁻ production. Representative FACS profiles of mitochondrial O₂⁻ level from cells after 24-hour culture in fresh, ZTG, HYSTEM®_{3D}, control, DXI, ZTG_{2D} and HYSTEM®_{2D} conditions.

FIGS. 26A-26C. Nonspecific interactions between HSPC and hydrogel matrix and reduced degree of oxygen in ZTG culture. (FIG. 26A) When the TAMRA fluorophoresis presented on the matrix, 480 nm excitation yields weak 590 nm (red) emission (‘Hydrogel solution’ sample). When the HSPCs membrane is labeled with 5-hexadecanoylaminofluorescein, 480 nm excitation yields 525 nm (green) emission (‘HSPC suspension’ sample), unless TAMRA labeled components are bound to HSPCs, in which case 525 nm emission is diminished and 580 nm (red) emission is enhanced (FRET effect). (FIG. 26B) Normalized FRET of HSPCs-encapsulated ZTG and HYSTEM® hydrogels. (FIG. 26C) Measured dissolved oxygen (DO) levels after 24-hour culture in each system. Asterisks designate statistical significance as per students' t-test performed between indicated groups; N=4, p<0.05.

FIGS. 27A-27C. ZTG culture avoids nonspecific pathway activation/deactivation. Mean fluorescent intensity of (FIG. 27A) phospho-p38, (FIG. 27B) phospho-mTOR and (FIG. 27C) β-Catenin were examined after 1-day culture. ns indicates no significant difference. * indicates a significant difference (P<0.05). Values represent means±SD, n=3 independent experiments.

FIGS. 28A-28E. ZTGₒₚₜ culture results in CD34+CB cells with reduced metabolic activity. Difference of (FIG. 28A) mitochondrial mass and (FIG. 28B) membrane potential of CB cells after 14- or 24-day ex vivo culture in each condition was presented by the mean fluorescent intensity of MitoTracker red and MitoTracker green. Percentage change of (FIG. 28C) glucose consumption and (FIG. 28D) lactate secretion by CB cells after ex vivo culture in each condition. (FIG. 28E) Amino acid metabolism of CB cells before and after culture in control, ZTGₒₚₜ and DXIₒₚₜ conditions. NS indicates no significant difference. * indicates a significant difference (P<0.05). Values represent means±SD, n=3 independent experiments.

FIGS. 29A, 29B. 14.3% genes changed significantly after ZTGₒₚₜ culture. (FIG. 29A) Volcano plots of statistical significance against fold-change between cord blood CD34⁺ HSPCs cultured in ZTGₒₚₜ and fresh cord blood CD34⁺ HSPCs demonstrating that 1,704 out of 11,912 genes are found to be significantly differentially expressed. There are 778 genes up-regulated (gray, right dots) and 926 genes down regulated (gray, left dots). More than 10,000 genes (black dots) did not show significant change after ZTGₒₚₜ culture. (FIG. 29B) Top 20 up- or down-regulated genes.

FIGS. 30A-30C. Gene ontology enrichment analysis of differentially expressed genes show statistically enriched GO categories for (FIG. 30A) down-regulated and (FIG. 30B) up-regulated GO terms. (FIG. 30C) Ratios of inhibited and activated pathways.

FIG. 31. Effect of ZTGₒₚₜ culture on canonical pathways. Significantly changed canonical pathways in ZTGₒₚₜ-cultured cells compared to fresh cells were analyzed by Ingenuity Pathway Analysis (IPA). The stacked bar chart displays the percentage of genes that were upregulated (left, gray portion of bar), downregulated (right, gray portion of bar), and genes not overlapping with the data set (white) in each canonical pathway. The numerical value at the top of each bar represents the total number of genes in the canonical pathway. The secondary y-axis (right) shows the −log of P-value calculated by the Fisher method, which indicates the significance of each pathway.

FIG. 32. Cell cycle analysis by FACS using anti-Ki-67 and Hoechst 33342 staining for HSPCs before and the cells after culture in each condition. Representative cell cycle profiles of cells in each condition and the percentage of cells in each gate.

FIG. 33. In ZTG, both CD34⁺ cell population and CD34⁺CD45RA⁻ cell populations were retained when compared to fresh cells. In contrast, reduced frequency of CD34+ cells and CD34⁺CD45RA⁻ cells were observed in Delta1^{ext-IgG} and TCPS culture systems.

FIG. 34. Flow cytometry histograms comparing various cell markers between CD34+ HSPC before (filled) and after (unfilled) ZTG culture. Fold expansion of total cell and primitive subpopulation were examined, and although total expanded cell number was low in ZTG culture, the primitive phenotypes and functionally defined cells suggested improved effects of ZTG in restraining the differentiation of CD34+ cells. This finding was also confirmed by the negligible fold expansion of CD34⁻ cells and low expression of differentiation markers in ZTG.

FIG. 35. Cell composition (CD34⁻ versus CD34⁺) before and after ZTG culture.

FIGS. 36A, 36B. (FIG. 36A) flow cytometry histograms showing the difference of several cell surface markers between cells before (filled) and after (unfilled) ZTG culture. Fresh cells show a cell composition of predominantly CD34⁻ over CD34⁺. However, ZTG-cultured HSCs show a cell composition of predominantly CD34⁺ over CD34. (36B) Data underlying the findings presented in FIG. 36A.

### DETAILED DESCRIPTION

Hematopoietic cell transplantation (HCT) involves transfer of stem cells and progenitor cells, usually derived from bone marrow, peripheral blood, or cord blood. Hematopoietic stem/progenitor cells (HSPCs) obtained from cord blood have been increasingly utilized for HCT, primarily because of their ready availability, less need for human leukocyte antigen (HLA) matching, and reduced occurrence of graft-versus-host disease (GVHD). However, the cell dose of cord blood-derived stem and progenitor cell populations is limited in a cord blood unit, which delays hematopoietic recovery and restricts wider application. For example, two units of cord blood from different donors generally are required for many patients to ensure adequate provision of hematopoietic cells for successful engraftment. Because of cell dose limitations, methods to expand hematopoietic stem cell (HSC) populations have been attempted.

Previous expansion methods, including cytokine-mediated expansion, resulted in proliferation of HSPC populations, but that proliferation was accompanied by significant differentiation and maturation, leading to loss of HSC functionality and a negative impact on long-term hematopoietic reconstitution. A number of other methodologies, including use of aryl hydrocarbon antagonists and Notch ligands, as well as attempts to optimize the balance of stimulatory and inhibitory factors in culture media have been attempted. However, while some degree of HSC expansion was noted with these methods, still significant progenitor cell generation and differentiation was present.

The current disclosure provides culture systems and methods to expand CD34+ hematopoietic stem/progenitor (HSPC) cell populations using ultra-low fouling hydrogels, such as zwitterionic hydrogels (ZTG). Expansion using these culture systems and methods results in expanded cell populations with increased proportion of HSC versus partially or fully differentiated cells, proportionally lower cell surface expression levels of differentiation/maturation markers, reduced metabolic rates, and/or a greater proportion of quiescent cells, as compared to currently available clinical expansion methods. Each of these characteristics is beneficial for cell populations expanded for research or therapeutic uses requiring long-term hematopoietic reconstitution. Cell populations with increased proportion of HSC versus partially or fully differentiated cells, proportionally lower cell surface expression levels of differentiation/maturation markers, reduced metabolic rates, and/or a greater proportion of quiescent cells as compared to CD34+ HSPC expanded using a relevant control condition as described herein are referred to as “ZTG-expanded HSC populations.”

In particular embodiments, the ZTG includes a zwitterionic polymer. In particular embodiments, the zwitterionic polymer includes a four-arm poly(carboxybetaine acrylamide) tetracyclooctyne. In particular embodiments, the ZTG includes a zwitterionic polymer crosslinker that is degraded by a product released by an expanding cell population. In particular embodiments, the crosslinker is a peptide. In particular embodiments, the peptide includes a poly(EK) crosslinker. In particular embodiments, the poly(EK) crosslinker includes a bis(azide) di-functionalized polypeptide. In particular embodiments, the bis(azide) di-functionalized polypeptide includes Azide-GG-(KE)₂₀-GPQGIWGQ-(KE)₂₀GG-Azide (SEQ ID NO: 1).

In particular embodiments, the culture systems and methods result in an increased proportion of HSC versus partially or fully differentiated cells following expansion as compared to a relevant control condition. Without being limited by theory, in particular embodiments, the increased proportion of stems cells versus partially or fully differentiated cells in ZTG-expanded HSC populations occurs due to inhibited differentiation during culture while promoting expansion and maintenance of cells expressing more primitive HSC markers/phenotypes.

In particular embodiments, the increased proportion of HSC versus partially or fully differentiated cells following expansion in a ZTG-expanded HSC population is demonstrated through having, at the end of expansion, at least a 10-fold increase, at least a 15-fold increase, at least a 25-fold increase, at least a 50-fold increase, at least a 100-fold increase, at least a 150-fold increase, or at least a 200-fold increase in the most phenotypically primitive subset of HSC. In particular embodiments, the most phenotypically primitive subset is CD34+, CD38−, CD45RA−, CD49f+, CD90+. In particular embodiments, an increased proportion of HSC versus partially or fully differentiated cells in a ZTG-expanded HSC population can be demonstrated by an increased proportion of CD34+, CD38−, CD45RA−, CD49f+, CD90+ cells following expansion as compared to cells following expansion in a relevant control condition. “At the end of expansion” and “following expansion” refers to the time when an expanding cell population is removed from culture conditions intended to promote expansion (or alternatively, the culture conditions intended to promote expansion are removed from the cell population).

In particular embodiments, the increased proportion of stems cells versus partially or fully differentiated cells in a ZTG-expanded HSC population is demonstrated through having, at the end of expansion, at least 50%, at least 60%, at least 70%, at least 80%, at least 90%, or at least 95% cells that are CD34+ and Lin−. Lin refers to “lineage markers” which can be used to identify the lineage of a mature, differentiated hematopoietic cell (e.g., a myeloid cell or a lymphoid cell). Examples of markers within the Lin grouping include CD7, CD14, CD15, CD19 and CD56. In particular embodiments, HSCs are negative for these Lin markers.

In particular embodiments, the increased proportion of HSC in a ZTG-expanded HSC population is demonstrated through having, at the end of expansion, at least 20%, at least 30%, at least 40%, at least 50%, at least 60%, at least 70%, or at least 85% cells that are CD34+ and CD45RA-negative. In particular embodiments, the increased proportion of stems cells versus partially or fully differentiated cells in a ZTG-expanded HSC population can be evidenced by a reduced presence of cells with granular cytoplasm and/or irregular nuclei, as compared to a relevant control condition.

In particular embodiments, reduced metabolic rates following expansion of a ZTG-expanded HSC population are demonstrated through reduced glucose consumption, reduced lactate secretion, and/or reduced amino acid metabolism as compared to a relevant control condition. In particular embodiments, changes in metabolic rate can be measured, for example, by quantifying metabolites using mass spectrometry.

In particular embodiments, the reduced metabolic rate in a ZTG-expanded HSC population following expansion can be associated with a down-regulation of one or more gene sets associated with (i) cell differentiation, (ii) cell activation, or (iii) cytokine production. In particular embodiments, expression of genes or gene sets can be measured by RNAseq. In particular embodiments, down-regulation can refer to a lower expression level of a gene or a set of genes as compared to a relevant control condition. In particular embodiments, gene sets or pathways can be determined as down-regulated, up-regulated, or unchanged using a gene ontology analysis tool, such as the GO enrichment analysis tool provided by the Gene Ontology Consortium.

In particular embodiments, a greater proportion of quiescent cells in a ZTG-expanded HSC population can be demonstrated against a relevant control condition. In particular embodiments, a quiescent cell can be a cell that is reversibly in the G₀ stage of the cell cycle. That is, a quiescent cell can be a cell that is in G₀ phase, but is able to enter the cell cycle again. In contrast, a cell may enter the G₀ stage of the cell cycle irreversibly, for example, through senescence or differentiation. Irreversible entry into the G₀ phase may occur, for example, if a cell undergoes DNA damage, such as due to reactive oxygen species (ROS). In particular embodiments G₁ may refer to the stage of the cell cycle following G₀, in which a cell may increase in size and prepare for DNA synthesis. In particular embodiments, S-G₂M (a combination of the Synthesis, G₂, and mitosis stages) may refer to the stage in which a cell replicates its DNA and continues to grow in preparation for cell division, and next separates its two identical sets of chromosome into two nuclei. In particular embodiments, the proportion of cells in G₀, G₁ and/or G₂-S/M can be measured by staining with anti-Ki-67 and Hoechst 33342. Ki-67 is a nuclear protein associated with cell proliferation. Resting, non-cycling cells (G₀ phase) have little to no Ki-67 expression. Hoechst 33342 is a stain that binds to nucleic acids, which are in higher abundance in cells in G₂-S/M, as compared to cells in G₀ or G₁. In particular embodiments, a ZTG-expanded HSC population may produce a cell population that is at least 40% quiescent (i.e., at least 40% of the cells in the cell population are in the G₀ stage), at least 70% quiescent, or at least 90% quiescent. In particular embodiments, a ZTG-expanded HSC population may produce a cell population that is at least 40% quiescent after 10 days of expanding, at least 70% quiescent after 12 days of expanding, or at least 90% quiescent after 14 days of expanding. In particular embodiments, quiescent cells expanded in a ZTG may enter the cell cycle again (i.e., move into G₁ phase) following transfer into a non-zwitterionic culture (e.g., a two-dimensional polystyrene flask). In particular embodiments, a quiescent HSC is capable of self-renewal.

In particular embodiments, cells within a ZTG-expanded HSC population can have reduced mitochondrial mass and/or reduced mitochondrial membrane potential, as compared to cells within a relevant control condition. In particular embodiments, mitochondrial membrane potential can refer to the electrical potential across the inner mitochondrial membrane. In particular embodiments, mitochondrial membrane potential can be assayed using a dye, such as MitoTracker Red, which stains mitochondria, and its accumulation is dependent upon membrane potential. In particular embodiments, mitochondrial mass can be measured with a dye, such as MitoTracker Green, which stains mitochondria, and its accumulation is dependent on the size of the mitochondria, but not the membrane potential.

In particular embodiments, expansion using a ZTG may lead to decreased production of ROS by the expanding cell population, as compared to cells expanding in a relevant control condition. Reactive oxygen species are chemically reactive chemical species that contain an oxygen, such as peroxides, superoxides, hydroxyl radical, and singlet oxygen. In particular embodiments, cellular stress may lead to increased production of ROS. ROS may cause cellular damage, and may induce cell differentiation, DNA damage, and/or apoptosis. Without being bound by theory, a hydrophobic culture (e.g., a two-dimensional polystyrene flask) may result in excessive ROS production due to changes in protein conformation as the proteins nonspecifically interact with the hydrophobic surface, leading to disturbance of cell membranes and induction of cellular ROS production. In particular embodiments, decreased production of ROS can be demonstrated by measuring cellular and mitochondrial ROS levels, such as with a horseradish peroxidase assay to measure hydrogen peroxide. In particular embodiments, decreased production of ROS can be demonstrated by: a decrease in phospho-p38 MAPK, a decrease in phosphor-mTOR, and/or an increase in beta-catenin, as compared to the levels of these in cells expanded using a relevant control condition.

A relevant control condition is one wherein CD34+ HSPC are expanded under comparable experimental procedures, but for the variable of interest. As is understood by one of ordinary skill in the art, comparable indicates that the experimental procedures are intended to match but may include some minor unavoidable or unintended discrepancies. Within the studies described herein, the variable of interest is the substrate on or in which CD34+ HSPC are expanded. One substrate is a ZTG as disclosed herein. A control substrate is a hydrophobic polystyrene flask for two-dimensional culture of cells. Hydrophobic polystyrene flasks are commercially available from, for example, Corning, Inc. Another control substrate utilizes a Notch agonist substrate. In particular embodiments, a control substrate is a hydrophobic polystyrene flask with a surface coated with a Notch agonist substrate. As used herein, a relevant control condition utilizing a Notch agonist refers to a flask pre-coated with Delta1^{ext-IgG} at 2.5 μg/mL (a density previously been shown optimal for generation of NOD/SCI D-repopulating cells), together with 5 μg/mL of fibronectin fragment CH-296 (Takara Shuzo Co. LTD) overnight at 4° C., washed with PBS, and then blocked with PBS-2% BSA or HSA at 37° C. See, Delaney et al., Nat Med, 16(2):232 (2010). As is understood by one of ordinary skill in the art, relevant control conditions begin with comparable cell starting populations.

ZTG-expanded HSC populations are useful to treat a wide variety of adverse conditions where a patient requires or would benefit from long-term hematopoietic reconstitution. In particular embodiments, cells within a ZTG-expanded HSC population are genetically modified to support a treatment against the adverse condition.

Aspects and options of the current disclosure are now described in more detail as follows: (i) Cell Populations; (ii) Hydrogels; (iii) Genetic Modifications; (iv) Compositions; (v) Kits; (vi) Methods of Use (vii) Exemplary Embodiments; and (viii) Experimental Examples.

### (i) Cell Populations

Hematopoietic stem cells (HSCs) can self-renew and can differentiate into (i) myeloid progenitor cells, which through continued differentiation ultimately give rise to monocytes and macrophages, neutrophils, basophils, eosinophils, erythrocytes, megakaryocytes/platelets, or dendritic cells; or (ii) lymphoid progenitor cells which ultimately give rise to T-cells, B-cells, and lymphocyte-like cells called natural killer cells (NK cells). For a general discussion of hematopoiesis and HSC differentiation, see Chapter 17, Differentiated Cells and the Maintenance of Tissues, Alberts et al., 1989, Molecular Biology of the Cell, 2nd Ed., Garland Publishing, New York, N.Y.; Chapter 2 of Regenerative Medicine, Department of Health and Human Services, Aug. 5, 2006, and Chapter 5 of Hematopoietic Stem Cells, 2009, Stem Cell Information, Department of Health and Human Services.

HSCs can be positive for a specific marker expressed in increased levels on HSC relative to other types of hematopoietic cells. For example, such markers include CD34, CD43, CD45RO, CD59, CD90, CD109, CD117, CD133, CD166, HLA DR, or a combination thereof. Also, HSCs can be negative for an expressed marker relative to other types of hematopoietic cells. For example, HSC can be negative for Lin marker groupings (which indicate differentiation), CD38, CD45RA or a combination thereof. Examples of markers within Lin groupings include CD7, CD14, CD15, CD19 and CD56. HSCs can be negative for these Lin markers. Preferably, cells of cell populations expanded with the teachings of the current disclosure are CD34+. In particular embodiments, an HSC can be CD34+ and CD38−, whereas an HSPC can be CD34+ and CD38+. As previously stated, the most primitive HSCs are associated with the CD34+, CD83−, CD45R−, CD90+, CD49f+ profile.

Sources of hematopoietic cell populations including HSPC are umbilical cord blood, placental blood, and peripheral blood (see U.S. Pat. Nos. 5,004,681; 7,399,633; and U.S. Pat. No. 7,147,626; Craddock et al., Blood, 90(12):4779 (1997); Jin et al., J Transl Med, 6:39 (2008); Pelus, Curr Opin Hematol, 15(4):285 (2008); Papayannopoulou et al., Blood, 91(7):2231 (1998); Tricot et al., Haematologica, 93(11):1739 (1998); and Weaver et al., Bone Marrow Transplantation, 27(2):523 (2001)). Methods regarding collection, anti-coagulation and processing, etc. of blood samples are well known in the art. See, for example, Alsever et al., NY St J Med, 41:126 (1941); De Gowin, et al., J Am Med Assoc, 114:850 (1940); Smith, et al., J Thorac Cardiovasc Surg, 38:573 (1959); Rous and Turner, J Exp Med, 23:219 (1916); and Hum, Storage of Blood, Academic Press, New York, pp. 26-160 (1968). Sources of HSPC populations also include bone marrow (see Kodo et al., J Clin Invest, 73:1377 (1984)), embryonic cells, aortal-gonadal-mesonephros derived cells, lymph, liver, thymus, and spleen from age-appropriate donors. All collected samples of cell populations can be screened for undesirable components and discarded, treated, or used according to accepted current standards at the time.

CD34+ hematopoietic cells can be collected and isolated from a sample using any appropriate technique. Appropriate collection and isolation procedures include, for example, magnetic separation; fluorescence-activated cell sorting (FACS; Williams et al., J Immunol, 135:1004 (1985); Lu et al., Blood, 68(1):126 (1986)); affinity chromatography; cytotoxic agents joined to a monoclonal antibody or used in conjunction with a monoclonal antibody, e.g., complement and cytotoxins; “panning” with antibody attached to a solid matrix (Broxmeyer et al., J Clin Invest, 73:939 (1984)); selective agglutination using a lectin such as soybean (Reisner et al., PNAS, 77:1164 (1980)); etc.

In particular embodiments, a sample (for example, a fresh cord blood unit) can be processed to select/enrich for CD34+ cells using anti-CD34 antibodies directly or indirectly conjugated to magnetic particles in connection with a magnetic cell separator, for example, the CLINIMACS® Cell Separation System (Miltenyi Biotec, Bergisch Gladbach, Germany). See also, sec. 5.4.1.1 of U.S. Pat. No. 7,399,633 which describes enrichment of CD34+ HSPC from 1-2% of a normal bone marrow cell population to 50-80% of the population.

Similarly, HSPCs expressing CD43, CD45RO, CD45RA, CD59, CD90, CD109, CD117, CD133, CD166, HLA DR, or a combination thereof, can be enriched for using antibodies against these antigens. U.S. Pat. No. 5,877,299 describes additional appropriate hematopoietic antigens that can be used to isolate, collect, and enrich HSPC cells from samples.

Once HSPC have been collected (and optionally isolated, such as by using the above techniques), expansion of the cells in an ultra-low fouling hydrogel, such as a ZTG, can be performed. In particular embodiments, in the process of gelation, CD34⁺ HSPC, may be added before or during gelation, and allowed to become encapsulated within the hydrogel as it forms.

### (ii) Hydrogels

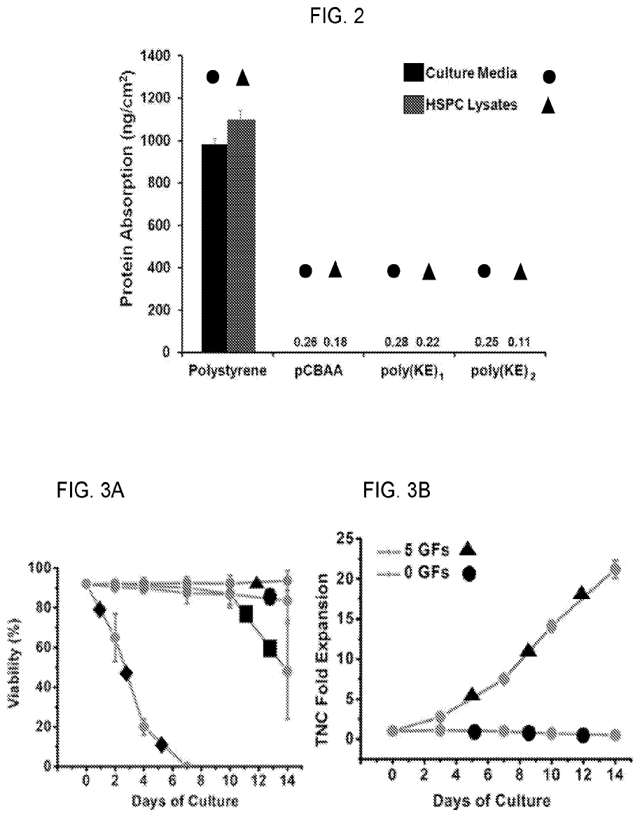

A “hydrogel” refers to a network of polymer chains that are hydrophilic in which water or an aqueous medium is the dispersion medium. Particular embodiments disclosed herein utilize a ZTG for expansion. Exemplary ZTG useful according to the disclosed methods are described in, for example, International Patent Publication No. WO2016/040489 A1. In particular embodiments, ZTG include a zwitterionic polymer and a biodegradable zwitterionic peptide used as a crosslinker. Zwitterionic refers to the property of overall charge neutrality while having both a positive and a negative electrical charge. As is understood by one of ordinary skill in the art, absolute charge neutrality is not required. In particular embodiments, a hydrogel is considered zwitterionic within the current disclosure if it shows ultra-low fouling in complex protein solutions in a manner that is not statistically significantly different than that as measured and demonstrated in FIG. 2.

A “zwitterionic polymer” refers to a polymer or copolymer having zwitterionic monomers. Zwitterionic monomers have pendant groups (i.e., groups pendant from the polymer backbone) that include zwitterionic groups. Representative zwitterionic pendant groups include carboxybetaine groups (e.g., —Ra-N+(Rb)(Rc)-Rd-CO₂⁻, where Ra is a linker group that covalently couples the polymer backbone to the cationic nitrogen center of the carboxybetaine groups, Rb and Rc are nitrogen substituents, and Rd is a linker group that covalently couples the cationic nitrogen center to the carboxy group of the carboxybetaine group).

The zwitterionic polymer or copolymer may be a “star polymer” or “star-shaped polymer,” which refers to a branched polymer in which two or more polymer branches extend from a core. Representative star polymers of the disclosure include two, three, four, five, six, or more branches extending from the core. The core is a group of atoms having two or more functional groups from which the branches can be extended by polymerization. Representative cores have two, three, four, five, six, or more functional groups from which the branches can be extended. In particular embodiments, the branches are zwitterionic polymeric branches.

For the star polymers of the present disclosure, the branches may be any zwitterionic polymers and their precursors that can be converted to zwitterionic polymers via hydrolysis, ultraviolet irradiation, or heat. The zwitterionic polymers may be obtained by any polymerization method effective for polymerization of unsaturated monomers, including atom transfer radical polymerization (ATRP), reversible addition-fragmentation chain transfer polymerization (RAFT), photo-polymerization, ring-opening polymerization (ROP), condensation, Michael addition, branch generation/propagation reaction, or other reactions. In particular embodiments, the polymers having terminal functional groups are able to specifically bind to a binding partner and at the same time avoid nonspecific biofouling, which is imparted to the polymers by their zwitterionic structures. By virtue of their functionalized terminal ends, the polymers of the present disclosure can be converted to further functionalized polymers useful for making hydrogels by complimentary coupling chemistries (e.g., click chemistries, thiol exchange reactions, reductive reactions, and other chemistries known in the art). In particular embodiments, the complimentary coupling chemistry used is bioorthogonal chemistry. The term bioorthogonal chemistry can refer to any chemical reaction that can occur in proximity of living cells without interfering with native biochemical processes. In particular embodiments, click chemistry is used to link a function group to the polymer. Click chemistry can refer to strain-promoted azide-alkyne cycloaddition (SPAAC). For example, an azide present on a functional group (e.g., a functional group including a metalloproteinase-cleavable motif) can react with an alkyne present in the polymer (e.g., a difluorinated cyclooctyne). These functional end groups can be pre- and post-modified after the branches are created.

In particular embodiments, the functionalized polymers or copolymers utilized within the present disclosure are prepared from polymerization of suitable polymerizable zwitterionic monomers. In particular embodiments, the polymer or copolymer has repeating units having formula (I):

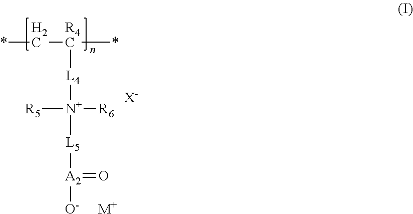

wherein
R₄ is selected from hydrogen, fluorine, trifluoromethyl, C₁-C₆ alkyl, and C₆-C₁₂ aryl groups;
R₅ and R₆ are independently selected from alkyl and aryl, or taken together with the nitrogen to which they are attached form a cationic center;
L₄ is a linker that covalently couples the cationic center [N+(R₅)(R₆)] to the polymer backbone [—(CH₂—CR₄)ₙ—];
L₅ is a linker that covalently couples the anionic center [A₂(O)O⁻] to the cationic center;
A₂ is C, SO, SO₂, P, or PO;
n is an integer from 5 to 10,000; and

* represents the point at which the repeating unit is covalently linked to an adjacent repeating unit or a functional group useful for forming hydrogels. Various functional groups that allow for polymerization of monomers and for click chemistry to take place could be used. As an example, the functional group (*) could be an acryloyl group,

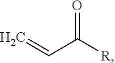

wherein R is O— or NH—. When R is an O, the monomer for the polymer having Formula I is in the form of a methacrylate monomer, and when R is NH, the monomer for the polymer having Formula I is in the form of a methacrylamide monomer.

In the polymer, the pendant zwitterionic groups can be internal salts and M⁺ and X⁻ can be absent.

In particular embodiments, R₄ is C₁-C₃ alkyl.

In particular embodiments, R₅ and R₆ are C₁-C₃ alkyl.

In particular embodiments, L₄ is selected from —C(O)O—(CH₂)ₚ— or —C(O)NH—(CH₂)ₚ—, wherein p is an integer from 1 to 20. In particular embodiments, L₄ as described above is —C(O)O—(CH₂)ₚ—, wherein p is 1-6.

In particular embodiments, L₅ is —(CH₂)_{q}—, where q is an integer from 1 to 20.

In particular embodiments, A₂ is C or SO.

In particular embodiments, n is an integer from 5 to 5,000.

In particular embodiments, R₄, R₅, and R₆ are methyl, L₄ is —C(O)O—(CH₂)₂—, L₅ is —(CH₂)—, A₁ is C, and n is an integer from 10 to 1,000.

The zwitterionic polymers utilized within the present disclosure can be prepared by polymerization of monomers having formula (II):

CH₂═C(R₄)-L₄-N⁺(R₅)(R₆)-L₅-A₂(O)O⁻ (II)

wherein R₄, R₅, R₆, L₄, L₅, and A₂, are as described above for the repeating unit of formula (I).

In particular embodiments, representative zwitterionic polymer branches utilized within the present disclosure have the formula (III):

PB-(L₄-N⁺(R₅)(R₆)-L₅-A₂(O)O⁻)ₙ (III)

wherein L₄, L₅, R₅, R₆, and A₂ are as described above for the repeating unit of formula (I), and PB is the polymer backbone of formula (I).

Additional examples of zwitterionic polymers include poly(carboxybetaine methacrylate); poly(phosphobetaine methacrylate); poly(sulfobetaine methacrylate); and poly(carboxymethyl betaine). See, for example, Sundaram et al., Advanced Materials Interfaces, 1(6):1400071 (2014) and Yang, et al., Acta Biomaterialia 40:92 (2016).

Particular embodiments of hydrogels utilized herein include a crosslinker that is degraded by a product released by an expanding cell population. Particular embodiments of hydrogels utilized herein include a crosslinker that, when degraded by a product released by an expanding cell population (e.g., a metalloproteinase), does not substantially affect cell growth or differentiation. For example, degradation of the crosslinker should not release products that substantially affect HSC growth or differentiation, or should not release significant amounts of products that substantially affect HSC growth or differentiation. In particular embodiments, biodegradable zwitterionic peptides can be used as crosslinkers. Examples include KE peptides and poly(EK) crosslinkers, such as a bis(azide) di-functionalized polypeptide. One example of a bis(azide) di-functionalized polypeptide Is Azide-GG-(KE)₂₀-GPQGIWGQ-(KE)₂₀GG-Azide (SEQ ID NO: 1). Additional examples of biodegradable zwitterionic peptides include glutamic acid; lysine; D-alanyl-D-alanine; and L-prolinylglycine.

Variants of peptides disclosed herein may also be used. “Variants” of peptides include those having one or more amino acid additions, deletions, stop positions, or substitutions, as compared to a peptide disclosed herein.

An amino acid substitution can be a conservative or a non-conservative substitution. Variants of peptides disclosed herein can include those having one or more conservative amino acid substitutions. A “conservative substitution” involves a substitution found in one of the following conservative substitutions groups: Group 1: alanine (Ala or A), glycine (Gly or G), Ser, Thr; Group 2: aspartic acid (Asp or D), glutamic acid (Glu or E); Group 3: asparagine (Asn or N), glutamine (Gln or Q); Group 4: arginine (Arg or R), lysine (Lys or K), histidine (His or H); Group 5: isoleucine (Ile or I), leucine (Leu or L), methionine (Met or M), valine (Val or V); and Group 6: phenylalanine (Phe or F), tyrosine (Tyr or Y), tryptophan (Trp or V.

Additionally, amino acids can be grouped into conservative substitution groups by similar function, chemical structure, or composition (e.g., acidic, basic, aliphatic, aromatic, sulfur-containing). For example, an aliphatic grouping may include, for purposes of substitution, Gly, Ala, Val, Leu, and Ile. Other groups containing amino acids that are considered conservative substitutions for one another include: sulfur-containing: Met and Cys; acidic: Asp, Glu, Asn, and Gln; small aliphatic, nonpolar or slightly polar residues: Ala, Ser, Thr, Pro, and Gly; polar, negatively charged residues and their amides: Asp, Asn, Glu, and Gln; polar, positively charged residues: His, Arg, and Lys; large aliphatic, nonpolar residues: Met, Leu, Ile, Val, and Cys; and large aromatic residues: Phe, Tyr, and Trp. Additional information is found in Creighton (1984) Proteins, W.H. Freeman and Company.

Variants of peptides disclosed herein also include sequences with at least 70% sequence identity, at least 80% sequence identity, at least 85% sequence, at least 90% sequence identity, at least 95% sequence identity, at least 96% sequence identity, at least 97% sequence identity, at least 98% sequence identity, or at least 99% sequence identity to a peptide disclosed herein.

“% sequence identity” refers to a relationship between two or more sequences, as determined by comparing the sequences. In the art, “identity” also means the degree of sequence relatedness between peptide sequences as determined by the match between strings of such sequences. “Identity” (often referred to as “similarity”) can be readily calculated by known methods, including those described in: Computational Molecular Biology (Lesk, A. M., ed.) Oxford University Press, N Y (1988); Biocomputing: Informatics and Genome Projects (Smith, D. W., ed.) Academic Press, N Y (1994); Computer Analysis of Sequence Data, Part I (Griffin, A. M., and Griffin, H. G., eds.) Humana Press, N J (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine sequence identity are designed to give the best match between the sequences tested. Methods to determine sequence identity and similarity are codified in publicly available computer programs. Multiple alignment of the sequences can be performed using the Clustal method of alignment (Higgins and Sharp CABIOS, 5, 151-153 (1989) with default parameters (GAP PENALTY=10, GAP LENGTH PENALTY=10). Relevant programs also include BLAST (Altschul, et al., J Mol Biol, 215:403 (1990); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y. Within the context of this disclosure it will be understood that where sequence analysis software is used for analysis, the results of the analysis are based on the “default values” of the program referenced. “Default values” mean any set of values or parameters which originally load with the software when first initialized.

In particular embodiments, cell populations may be encapsulated within zwitterionic poly(carboxybetaine) (pCB) polymers and zwitterionic KE peptides to yield a three-dimensional hydrogel that exhibits ultra-low fouling against either proteins in culture media or HSPC lysates. “Fouling” generally describes the unwanted buildup or adsorption of materials on the surface of cells. “Ultra-low fouling” levels relate to surfaces that are capable of repelling the accumulation of unwanted materials down to less than 20 ng/cm², less than 15 ng/cm², 10 ng/cm², 5 ng/cm², or less than 3 ng/cm². In particular embodiments, encapsulating cells can include crosslinking the hydrogel (e.g., ZTG) in the presence of the cells. See FIG. 9 for an example of cells encapsulated in ZTG. In particular embodiments, cell populations may be encapsulated and expanded in an ultra-low fouling hydrogel with minimal non-specific interaction.

### (iii) Genetic Modifications

Many of the conditions that can be treated with ZTG-expanded HSC populations can benefit by incorporating genetically-modified cells in, for example, a ZTG-expanded HSC population. In particular embodiments, any nucleic acid including a therapeutic gene (e.g., encoding a therapeutic protein) can be introduced into cells at any point during an expansion protocol. In particular embodiments, therapeutic genes are inserted before expansion.

The term “gene” or “therapeutic gene” refers to a nucleic acid sequence (used interchangeably with polynucleotide or nucleotide sequence) that encodes one or more therapeutic proteins as described herein. This definition includes various sequence polymorphisms, mutations, and/or sequence variants wherein such alterations do not substantially affect the function of the encoded one or more therapeutic proteins. The term “gene” may include not only coding sequences but also regulatory regions such as promoters, enhancers, and termination regions. The term further can include all introns and other DNA sequences spliced from the mRNA transcript, along with variants resulting from alternative splice sites. Gene sequences encoding the molecule can be DNA or RNA that directs the expression of the one or more therapeutic proteins. These nucleic acid sequences may be a DNA strand sequence that is transcribed into RNA or an RNA sequence that is translated into protein. The nucleic acid sequences include both the full-length nucleic acid sequences as well as non-full-length sequences derived from the full-length protein. The sequences can also include degenerate codons of the native sequence or sequences that may be introduced to provide codon preference in a specific cell type.

A gene sequence encoding one or more therapeutic proteins can be readily prepared by synthetic or recombinant methods from the relevant amino acid sequence. In particular embodiments, the gene sequence encoding any of these sequences can also have one or more restriction enzyme sites at the 5′ and/or 3′ ends of the coding sequence in order to provide for easy excision and replacement of the gene sequence encoding the sequence with another gene sequence encoding a different sequence. In particular embodiments, the gene sequence encoding the sequences can be codon optimized for expression in mammalian cells.

A “vector” is a nucleic acid molecule that is capable of transporting another nucleic acid. Vectors may be, e.g., viruses, phage, a DNA vector, a RNA vector, a viral vector, a bacterial vector, a plasmid vector, a cosmid vector, and an artificial chromosome vector. An “expression vector” is any type of vector that is capable of directing the expression of a protein encoded by one or more genes carried by the vector when it is present in the appropriate environment.

Viral vectors are usually non-replicating or replication-impaired vectors, which means that the viral vector cannot replicate to any significant extent in normal cells (e.g., normal human cells), as measured by conventional means (e.g. via measuring DNA synthesis and/or viral titer). Non-replicating or replication-impaired vectors may have become so naturally (i.e., they have been isolated as such from nature) or artificially (e.g., by breeding in vitro or by genetic manipulation). There will generally be at least one cell-type in which the replication-impaired viral vector can be grown—for example, modified vaccinia Ankara (MVA) can be grown in CEF cells. Typically, viral vectors are incapable of causing a significant infection in a subject, typically in a mammalian subject.

“Retroviruses” are viruses having an RNA genome. In particular embodiments, a retroviral vector contains all of the cis-acting sequences necessary for the packaging and integration of the viral genome, i.e., (a) a long terminal repeat (LTR), or portions thereof, at each end of the vector; (b) primer binding sites for negative and positive strand DNA synthesis; and (c) a packaging signal, necessary for the incorporation of genomic RNA into virions. More detail regarding retroviral vectors can be found in Boesen, et al., Biotherapy, 6:291 (1994); Clowes, et al., J Clin Invest, 93:644 (1994); Kiem, et al., Blood, 83:1467 (1994); Salmons and Gunzberg, Human Gene Therapy, 4:129 (1993); Miller, et al., Meth Enzymol, 217:581 (1993); and Grossman and Wilson, Curr Opin in Genetics and Devel, 3:110 (1993).

“Gammaretroviruses” refers to a genus of the retroviridae family. Exemplary gammaretroviruses include mouse stem cell virus, murine leukemia virus, feline leukemia virus, feline sarcoma virus, and avian reticuloendotheliosis viruses.

Widely used retroviral vectors include those based upon murine leukemia virus (MuLV), gibbon ape leukemia virus (GaLV), simian immunodeficiency virus (SIV), human immunodeficiency virus (HIV), and combinations thereof (see, e.g., Buchscher et al., J Virol, 66:2731 (1992); Johann et al., J Virol, 66:1635 (1992); Sommerfelt et al., Virol, 176:58 (1990); Wilson et al., J Virol, 63:2374 (1989); Miller et al., J Virol, 65:2220 (1991); and PCT/US94/05700).

Particularly suitable are lentiviral vectors. “Lentivirus” refers to a genus of retroviruses that are capable of infecting dividing and non-dividing cells and typically produce high viral titers. Lentiviral vectors have been employed in gene therapy for a number of diseases. For example, hematopoietic gene therapies using lentiviral vectors or gamma retroviral vectors have been used for x-linked adrenoleukodystrophy and beta thalassemia. See, e.g., Kohn et al., Clin Immunol, 135:247 (2010); Cartier et al., Methods Enzymol, 507:187 (2012); and Cavazzana-Calvo et al., Nature, 467:318 (2010). Several examples of lentiviruses include HIV (human immunodeficiency virus: including HIV type 1, and HIV type 2); equine infectious anemia virus; feline immunodeficiency virus (FIV); bovine immune deficiency virus (BIV); and simian immunodeficiency virus (SIV).

In particular embodiments, other retroviral vectors can be used in the practice of the methods of the invention. These include, e.g., vectors based on human foamy virus (HFV) or other viruses in the Spumavirus genera.

Foamy viruses (FVes) are the largest retroviruses known today and are widespread among different mammals, including all non-human primate species, however are absent in humans. This complete apathogenicity qualifies FV vectors as ideal gene transfer vehicles for genetic therapies in humans and clearly distinguishes FV vectors as gene delivery system from HIV-derived and also gammaretrovirus-derived vectors.

FV vectors are suitable for gene therapy applications because they can (1) accommodate large transgenes (>9 kb), (2) transduce slowly dividing cells efficiently, and (3) integrate as a provirus into the genome of target cells, thus enabling stable long term expression of the transgene(s). FV vectors do need cell division for the pre-integration complex to enter the nucleus, however the complex is stable for at least 30 days and still infective. The intracellular half-life of the FV pre-integration complex is comparable to the one of lentiviruses and significantly higher than for gammaretroviruses, therefore FV are also—similar to LV vectors—able to transduce rarely dividing cells. FV vectors are natural self-inactivating vectors and characterized by the fact that they seem to have hardly any potential to activate neighboring genes. In addition, FV vectors can enter any cells known (although the receptor is not identified yet) and infectious vector particles can be concentrated 100-fold without loss of infectivity due to a stable envelope protein. FV vectors achieve high transduction efficiency in pluripotent HSC and have been used in animal models to correct monogenetic diseases such as leukocyte adhesion deficiency (LAD) in dogs and Fanconi anemia in mice. FV vectors are also used in preclinical studies of β-thalassemia.

Additional examples of viral vectors include those derived from adenoviruses (e.g., adenovirus 5 (Ad5), adenovirus 35 (Ad35), adenovirus 11 (Ad11), adenovirus 26 (Ad26), adenovirus 48 (Ad48) or adenovirus 50 (Ad50)), adeno-associated virus (AAV; see, e.g., U.S. Pat. No. 5,604,090; Kay et al., Nat Genet, 24:257 (2000); Nakai et al., Blood, 91:4600 (1998)), alphaviruses, cytomegaloviruses (CMV), flaviviruses, herpes viruses (e.g., herpes simplex), influenza viruses, papilloma viruses (e.g., human and bovine papilloma virus; see, e.g., U.S. Pat. No. 5,719,054), poxviruses, vaccinia viruses, etc. See Kozarsky and Wilson, Current Opinion in Genetics and Development, 3:499 (1993), Rosenfeld, et al., Science, 252:431 (1991); Rosenfeld, et al., Cell, 68:143 (1992); Mastrangeli, et al., J Clin Invest, 91:225 (1993); Walsh, et al., Proc Soc Exp Bioi Med, 204:289 (1993); and Lundstrom, J Recept Signal Transduct Res, 19:673 (1999). Examples include modified vaccinia Ankara (MVA) and NYVAC, or strains derived therefrom. Other examples include avipox vectors, such as a fowlpox vectors (e.g., FP9) or canarypox vectors (e.g., ALVAC and strains derived therefrom).

Other methods of gene delivery include use of artificial chromosome vectors such as mammalian artificial chromosomes (Vos, Curr Op Genet Dev, 8:351 (1998)) and yeast artificial chromosomes (YAC). YAC are typically used when the inserted nucleic acids are too large for more conventional vectors (e.g., greater than 12 kb).

The CRISPR-Cas technology has been exploited to inactivate genes in human cell lines and cells. As an example, the CRISPR-Cas9 system, which is based on the type II system, has been used as an agent for genome editing.

The type II system requires three components: Cas9, crRNA, and tracrRNA. The system can be simplified by combining tracrRNA and crRNA into a single synthetic single guide RNA (sgRNA). An sgRNA can include a twenty nucleotide sequence that is complementary to a target sequence (analogous to the crRNA), and a tracrRNA sequence. For certain CRISPR-Cas9 systems, the target sequence may be adjacent to a PAM (e.g., 5′-20nt target-NGG-3′).

At least three different Cas9 nucleases have been developed for genome editing. The first is the wild type Cas9 which introduces double strand breaks (DSBs) at a specific DNA site, resulting in the activation of DSB repair machinery. DSBs can be repaired by non-homologous end joining (NHEJ), homology-directed repair (HDR), or microhomology mediated end joining (MMEJ). NHEJ can involve repair of a DSB with no homology (<5 bp) between the two ends joined during repair; HDR can involve repair of a DSB with a large region of homology between the ends joined during repair (100 or more nucleotides); and MMEJ can involve repair of a DSB with a small (5 to 50 bp) region of homology between the ends joined during repair. Another type of Cas9 that can be used is a mutant Cas9, known as the Cas9D10A, with only nickase activity, which means that it only cleaves one DNA strand and does not activate NHEJ. Thus, the DNA repairs proceed via the HDR pathway only. The third is a nuclease-deficient Cas9 (dCas9) which does not have cleavage activity but is able to bind DNA. Therefore, dCas9 is able to target specific sequences of a genome without cleavage. By fusing dCas9 with various effector domains, dCas9 can be used either as a gene silencing or activation tool.

Particular embodiments can utilize transcription activator-like effector nucleases (TALENs) as gene editing agents. TALENs refer to fusion proteins including a transcription activator-like effector (TALE) DNA binding protein and a DNA cleavage domain. TALENs are used to edit genes and genomes by inducing DSBs in the DNA, which induce repair mechanisms in cells. Generally, two TALENs must bind and flank each side of the target DNA site for the DNA cleavage domain to dimerize and induce a DSB. The DSB is repaired in the cell by NHEJ or by homologous recombination (HR) with an exogenous double-stranded donor DNA fragment.

As indicated, TALENs have been engineered to bind a target sequence of, for example, an endogenous genome, and cut DNA at the location of the target sequence. The TALEs of TALENs are DNA binding proteins secreted by Xanthomonas bacteria. The DNA binding domain of TALEs include a highly conserved 33 or 34 amino acid repeat, with divergent residues at the 12th and 13th positions of each repeat. These two positions, referred to as the Repeat Variable Diresidue (RVD), show a strong correlation with specific nucleotide recognition. Accordingly, targeting specificity can be improved by changing the amino acids in the RVD and incorporating nonconventional RVD amino acids.

Examples of DNA cleavage domains that can be used in TALEN fusions are wild-type and variant Fokl endonucleases. The Fokl domain functions as a dimer requiring two constructs with unique DNA binding domains for sites on the target sequence. The Fokl cleavage domain cleaves within a five or six bp spacer sequence separating the two inverted half-sites.

Particular embodiments utilize MegaTALs as gene editing agents. MegaTALs have a single chain rare-cleaving nuclease structure in which a TALE is fused with the DNA cleavage domain of a meganuclease. Meganucleases, also known as homing endonucleases, are single peptide chains that have both DNA recognition and nuclease function in the same domain. In contrast to the TALEN, the megaTAL only requires the delivery of a single peptide chain for functional activity.

Particular embodiments utilize zinc finger nucleases (ZFNs) as gene editing agents. ZFNs are a class of site-specific nucleases engineered to bind and cleave DNA at specific positions. ZFNs are used to introduce DSBs at a specific site in a DNA sequence which enables the ZFNs to target unique sequences within a genome in a variety of different cells. Moreover, subsequent to double-stranded breakage, HR or NHEJ takes place to repair the DSB, thus enabling genome editing.

ZFNs are synthesized by fusing a zinc finger DNA-binding domain to a DNA cleavage domain. The DNA-binding domain includes three to six zinc finger proteins which are transcription factors. The DNA cleavage domain includes the catalytic domain of, for example, Fokl endonuclease.

Vectors and other methods to deliver nucleic acids can include regulatory sequences to control the expression of the nucleic acid molecules. These regulatory sequences can be eukaryotic or prokaryotic in nature. In particular embodiments, the regulatory sequence can be a tissue specific promoter such that the expression of the one or more therapeutic proteins will be substantially greater in the target tissue type compared to other types of tissue. In particular embodiments, the regulatory sequence can result in the constitutive expression of the one or more therapeutic proteins upon entry of the vector into the cell. Alternatively, the regulatory sequences can include inducible sequences. Inducible regulatory sequences are well known to those skilled in the art and are those sequences that require the presence of an additional inducing factor to result in expression of the one or more therapeutic proteins. Examples of suitable regulatory sequences include binding sites corresponding to tissue-specific transcription factors based on endogenous nuclear proteins, sequences that direct expression in a specific cell type, the lac operator, the tetracycline operator and the steroid hormone operator. Any inducible regulatory sequence known to those of skill in the art may be used.

In particular embodiments, the nucleic acid is stably integrated into the genome of a cell. In particular embodiments, the nucleic acid is stably maintained in a cell as a separate, episomal segment.

In particular embodiments, the efficiency of integration, the size of the DNA sequence that can be integrated, and the number of copies of a DNA sequence that can be integrated into a genome can be improved by using transposons. Transposons or transposable elements include a short nucleic acid sequence with terminal repeat sequences upstream and downstream. Active transposons can encode enzymes that facilitate the excision and insertion of nucleic acid into a target DNA sequence.

A number of transposable elements have been described in the art that facilitate insertion of nucleic acids into the genome of vertebrates, including humans. Examples include sleeping beauty (e.g., derived from the genome of salmonid fish); piggyback (e.g., derived from lepidopteran cells and/or the Myotis lucifugus); mariner (e.g., derived from Drosophila); frog prince (e.g., derived from Rana pipiens); Tol2 (e.g., derived from medaka fish); TcBuster (e.g., derived from the red flour beetle Tribolium castaneum) and spinON. CRISPR-Cas systems may also be used.

Examples of particular therapeutic genes to treat particular adverse conditions are described below in the Methods of Use section.

### (iv) Compositions

ZTG-expanded HSC populations (non-genetically modified or genetically modified) can be prepared as compositions for administration to a subject. A composition refers to a ZTG-expanded HSC population prepared with a pharmaceutically acceptable carrier for administration to a subject.

At various points during preparation of a composition, it can be necessary or beneficial to cryopreserve a cell population (e.g., CD34+ HSPC before expansion) or a ZTG-expanded HSC population (after expansion). The terms “frozen/freezing” and “cryopreserved/cryopreserving” can be used interchangeably. Freezing includes freeze drying.

As is understood by one of ordinary skill in the art, the freezing of cells can be destructive (see Mazur, Cryobiology, 14:251 (1977)) but there are numerous procedures available to prevent such damage. For example, damage can be avoided by (a) use of a cryoprotective agent, (b) control of the freezing rate, and/or (c) storage at a temperature sufficiently low to minimize degradative reactions. Exemplary cryoprotective agents include dimethyl sulfoxide (DMSO) (Lovelock and Bishop, Nature, 183:1394 (1959); Ashwood-Smith, Nature, 190:1204 (1961)), glycerol, polyvinylpyrrolidine (Rinfret, Ann. N.Y. Acad. Sci., 85:576 (1960)), polyethylene glycol (Sloviter and Ravdin, Nature, 196:548 (1962)), albumin, dextran, sucrose, ethylene glycol, i-erythritol, D-ribitol, D-mannitol (Rowe et al., Fed Proc, 21:157 (1962)), D-sorbitol, i-inositol, D-lactose, choline chloride (Bender et al., J Appl Physiol, 15:520 (1960)), amino acids (Phan The Tran and Bender, Exp Cell Res, 20:651 (1960)), methanol, acetamide, glycerol monoacetate (Lovelock, Biochem J, 56:265 (1954)), and inorganic salts (Phan The Tran and Bender, Proc Soc Exp Biol Med, 104:388 (1960); Phan The Tran and Bender, in Radiobiology, Proceedings of the Third Australian Conference on Radiobiology, Ilbery ed., Butterworth, London, p. 59 (1961)). In particular embodiments, DMSO can be used. Addition of plasma or serum (e.g., to a concentration of 5-70%) can augment the protective effects of DMSO. After addition of DMSO, cells can be kept at 0° C. until freezing, because DMSO concentrations of 1% can be toxic at temperatures 10° C. or above.

In the cryopreservation of cells, slow controlled cooling rates can be critical and different cryoprotective agents (Rapatz et al., Cryobiology, 5(1):18 (1968)) and different cell types have different optimal cooling rates (see e.g., Rowe and Rinfret, Blood, 20:636 (1962); Rowe, Cryobiology, 3(1):12 (1966); Lewis, et al., Transfusion, 7(1):17 (1967); and Mazur, Science, 168:939 (1970) for effects of cooling velocity on survival of HSC and on their transplantation potential). The heat of fusion phase where water turns to ice should be minimal. The cooling procedure can be carried out by use of, e.g., a programmable freezing device or a methanol bath procedure. Programmable freezing apparatuses allow determination of optimal cooling rates and facilitate standard reproducible cooling.

In particular embodiments, DMSO-treated cells can be pre-cooled on ice and transferred to a tray containing chilled methanol which is placed, in turn, in a mechanical refrigerator (e.g., Harris or Revco) at −80° C. Thermocouple measurements of the methanol bath and the samples indicate a cooling rate of 1 to 3° C./minute can be preferred. After at least two hours, the specimens can have reached a temperature of −80° C. and can be placed directly into liquid nitrogen (−196° C.).

After thorough freezing, the cells can be rapidly transferred to a long term cryogenic storage vessel. In particular embodiments, samples can be cryogenically stored in liquid nitrogen (−196° C.) or vapor (−1° C.). Such storage is facilitated by the availability of highly efficient liquid nitrogen refrigerators.

Further considerations and procedures for the manipulation, cryopreservation, and long term storage of cells, can be found in the following exemplary references: U.S. Pat. Nos. 4,199,022; 3,753,357; and 4,559,298; Gorin, Clinics In Haematology, 15(1):19 (1986); Bone-Marrow Conservation, Culture and Transplantation, Proceedings of a Panel, Moscow, July 22-26, International Atomic Energy Agency, Vienna, pp. 107-186 (1968); Livesey and Linner, Nature, 327:255 (1987); Linner et al., J Histochem Cytochem, 34(9):1123 (1986); and Simione, J Parenter Sci Technol, 46(6):226 (1992)).

Following cryopreservation, frozen cells can be thawed for use in accordance with methods known to those of ordinary skill in the art. Frozen cells are preferably thawed quickly and chilled immediately upon thawing. In particular embodiments, the vial containing the frozen cells can be immersed up to its neck in a warm water bath; gentle rotation will ensure mixing of the cell suspension as it thaws and increase heat transfer from the warm water to the internal ice mass. As soon as the ice has completely melted, the vial can be immediately placed on ice.

In particular embodiments, methods can be used to prevent cellular clumping during thawing. Exemplary methods include: the addition before and/or after freezing of DNase (Spitzer et al., Cancer, 45:3075 (1980)), low molecular weight dextran and citrate, hydroxyethyl starch (Stiff et al., Cryobiology, 20:17 (1983)), etc.

As is understood by one of ordinary skill in the art, if a cryoprotective agent that is toxic to humans is used, it should be removed prior to therapeutic use. Small amounts of DMSO are permitted; serious toxicity is avoided by minimizing the amount of exposure (e.g., cryopreserved cell infusions typically limit DMSO to <10 mL/kg of patient weight).

Exemplary carriers and modes of administration of cells are described at pages 14-15 of U.S. Patent Publication No. 2010/0183564. Additional pharmaceutical carriers are described in Remington: The Science and Practice of Pharmacy, 21st Edition, David B. Troy, ed., Lippincott Williams & Wilkins (2005).

In particular embodiments, cells can be harvested from a culture medium, and washed and concentrated into a carrier in a therapeutically-effective amount. Exemplary carriers include saline, buffered saline, physiological saline, water, Hanks' solution, Ringer's solution, NORMOSOL-R® (Abbott Laboratories, Corp., Chicago, Ill.), PLASMA-LYTE A® (Baxter Laboratories, Inc., Morton Grove, Ill.), glycerol, and combinations thereof.

In particular embodiments, carriers can be supplemented with human serum albumin (HSA) or other human serum components or fetal bovine serum. In particular embodiments, a carrier for infusion includes buffered saline with 5% HSA or dextrose. Additional isotonic agents include polyhydric sugar alcohols including trihydric or higher sugar alcohols, such as glycerin, erythritol, arabitol, xylitol, sorbitol, or mannitol.

Carriers can include buffering agents, such as citrate buffers, succinate buffers, tartrate buffers, fumarate buffers, gluconate buffers, oxalate buffers, lactate buffers, acetate buffers, phosphate buffers, histidine buffers, and/or trimethylamine salts.

Stabilizers refer to a broad category of excipients which can range in function from a bulking agent to an additive which helps to prevent cell adherence to container walls. Typical stabilizers can include polyhydric sugar alcohols; amino acids, such as arginine, lysine, glycine, glutamine, asparagine, histidine, alanine, ornithine, L-leucine, 2-phenylalanine, glutamic acid, and threonine; organic sugars or sugar alcohols, such as lactose, trehalose, stachyose, mannitol, sorbitol, xylitol, ribitol, myoinositol, galactitol, glycerol, and cyclitols, such as inositol; PEG; amino acid polymers; sulfur-containing reducing agents, such as urea, glutathione, thioctic acid, sodium thioglycolate, thioglycerol, alpha-monothioglycerol, and sodium thiosulfate; low molecular weight polypeptides (i.e., <10 residues); proteins such as HSA, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran.

Where necessary or beneficial, compositions can include a local anesthetic such as lidocaine to ease pain at a site of injection.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol, and 3-pentanol.

Therapeutically effective amounts of cells within compositions can be greater than 10² cells, greater than 10³ cells, greater than 10⁴ cells, greater than 10⁵ cells, greater than 10⁶ cells, greater than 10⁷ cells, greater than 10⁸ cells, greater than 10⁹ cells, greater than 10¹⁰ cells, or greater than 10¹¹ cells.

In compositions disclosed herein, cells are generally in a volume of a liter or less, 500 mL or less, 250 mL or less, or 100 mL or less. Hence the density of administered cells is typically greater than 10⁴ cells/mL, 10⁷ cells/mL, or 10⁸ cells/mL.

The compositions disclosed herein can be prepared for administration by, for example, injection, infusion, perfusion, or lavage. The compositions can further be formulated for bone marrow, intravenous, intradermal, intraarterial, intranodal, intralymphatic, intraperitoneal, intralesional, intraprostatic, intravaginal, intrarectal, topical, intrathecal, intratumoral, intramuscular, intravesicular, and/or subcutaneous injection.

In particular embodiments, the cell product can be prepared using containers suitable for use according to the method of administration and compatible with the method of storage, e.g., vials, ampoules, infusion bags, etc. In particular embodiments, cell product can be prepared using bags designed for cryogenic storage of blood products, e.g., CRYOSTORE® (Origen Biomedical, Corp., Austin, Tex.) bags in various capacities from 10 to 500 mL. Accordingly, the product can include, for example, from 10 million to 1,000 million viable CD34+ cells/bag, e.g., 100, 300, or 800 million viable CD34+ cells/bag.

Kits. Kits can include one or more containers including one or more hydrogels, ZTG, components of hydrogels, components of ZTG, zwitterionic polymers, components of zwitterionic polymers, peptides, crosslinkers, CD34+ hematopoietic cells, therapeutic genes, vectors, guideRNAs, and/or compositions described herein. In particular embodiments, the kits can include one or more containers containing one or more CD34+ hematopoietic cells, compositions and/or compositions to be used in combination with other cells or compositions. Associated with such container(s) can be a notice in the form prescribed by a governmental agency regulating the manufacture, use, or sale of pharmaceuticals or biological products, which notice reflects approval by the agency of manufacture, use, or sale for human use and/or administration. The notice may state that the provided cells or compositions can be administered to a subject without immunological matching. The kits can include further instructions for using the kit, for example, instructions regarding preparation of ultra-low fouling hydrogels, ZTG, zwitterionic polymers, cells expansion, composition formulation and/or use of any of these components; proper disposal of related waste; and the like. The instructions can be in the form of printed instructions provided within the kit or the instructions can be printed on a portion of the kit itself. Instructions may be in the form of a sheet, pamphlet, brochure, CD-ROM, or computer-readable device, or can provide directions to instructions at a remote location, such as a website. In particular embodiments, kits can also include some or all of the necessary medical supplies needed to use the kit effectively, such as flasks, buffers, cytokines, expansion media, syringes, ampules, tubing, facemask, a needleless fluid transfer device, an injection cap, sponges, sterile adhesive strips, Chloraprep, gloves, and the like. Variations in contents of any of the kits described herein can be made.

### (vi) Methods of Use

Methods of using compositions according to the methods and systems disclosed herein include treating subjects (humans, veterinary animals (dogs, cats, reptiles, birds, etc.), livestock (horses, cattle, goats, pigs, chickens, etc.), and research animals (monkeys, rats, mice, fish, etc.). Treating subjects includes delivering therapeutically effective amounts. Therapeutically effective amounts include those that provide effective amounts, prophylactic treatments, and/or therapeutic treatments.

An “effective amount” is the number of cells necessary to result in a desired physiological change in a subject. Effective amounts are often administered for research purposes. Effective amounts disclosed herein do one or more of: (i) provide blood support by reducing immunodeficiency, pancytopenia, neutropenia and/or leukopenia (e.g., repopulating cells of the immune system and (ii) provide long-term hematopoietic reconstitution.

A “prophylactic treatment” includes a treatment administered to a subject who does not display signs or symptoms of a condition to be treated or displays only early signs or symptoms of the condition to be treated such that treatment is administered for the purpose of diminishing, preventing, or decreasing the risk of developing the condition. Thus, a prophylactic treatment functions as a preventative treatment against a condition.

A “therapeutic treatment” includes a treatment administered to a subject who displays symptoms or signs of a condition and is administered to the subject to reduce the severity or progression of the condition.

The actual dose amount administered to a subject can be determined by a physician, veterinarian, or researcher taking into account parameters such as, for example, physical and physiological factors including target; body weight; type of condition; severity of condition; upcoming relevant events, when known; previous or concurrent therapeutic interventions; idiopathy of the subject; and route of administration. In addition, in vitro and in vivo assays can optionally be employed to help identify optimal dosage ranges.

Therapeutically effective amounts to administer can include greater than 10² cells, greater than 10³ cells, greater than 10⁴ cells, greater than 10⁵ cells, greater than 10⁶ cells, greater than 10⁷ cells, greater than 10⁸ cells, greater than 10⁹ cells, greater than 10¹⁰ cells, or greater than 10¹¹.

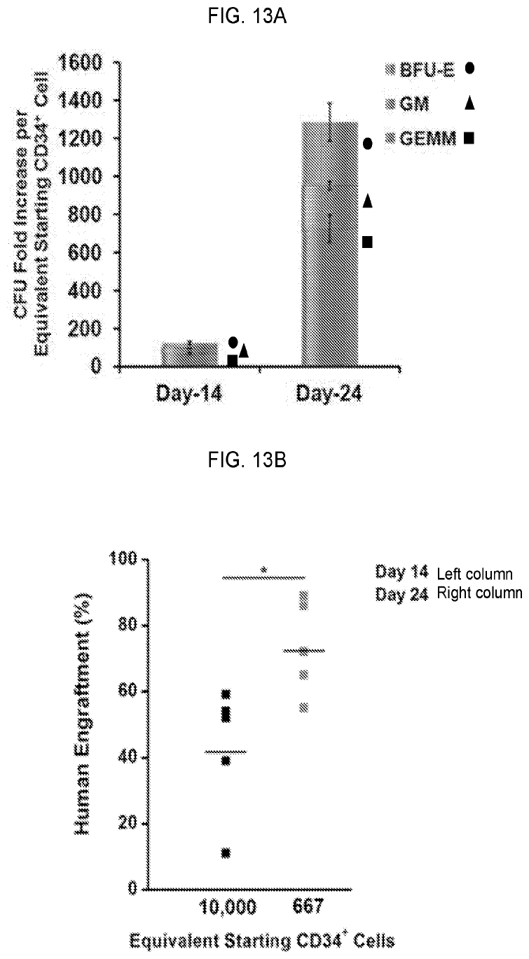

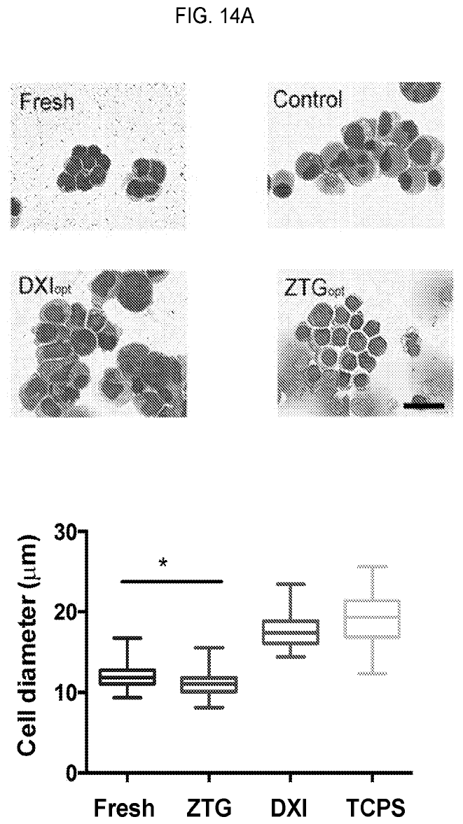

In particular embodiments, therapeutically effective amounts can provide hematopoietic reconstitution (i.e., hematopoietic repopulation). Hematopoietic reconstitution can refer to short-term and/or long-term hematopoietic reconstitution. Short-term hematopoietic reconstitution can refer to repopulation of a subject's hematopoietic cells within a subject for a period of less than 6 weeks after administration. Long-term hematopoietic reconstitution can refer to repopulation of a subject's hematopoietic cells at least 20 weeks after administration. In particular embodiments, ZTG-expanded HSC populations are capable of long-term multi-lineage reconstitution. In particular embodiments, multi-lineage reconstitution refers to reconstitution of more than one lineage of hematopoietic cells (e.g., myeloid cells and lymphoid cells). Cells that can provide long-term hematopoietic reconstitution can be referred to as long-term HSC. In particular embodiments, systems and methods disclosed herein may be advantageous over other state-of-the-art expansion techniques for expansion of cells to provide long-term hematopoietic reconstitution. Without being bound by theory, the advantages of the hydrogel-based expansion techniques may be due to the presence of a greater proportion of stems cells versus partially or fully differentiated cells in ZTG-expanded HSC populations, as compared to cell products expanded using other relevant control conditions. Evidence of long-term reconstitution and methods to assess the same are described in relation to FIGS. 13A, 13B, 17B-17F and 22.

In the context of blood support, therapeutically effective amounts treat immunodeficiency, pancytopenia, neutropenia and/or leukopenia by increasing the number of desired cells in a subject's circulation. Increasing the number of desired cells in a subject's circulation can re-populate the subject's immune system by increasing the number of immune system cells and/or immune system cell progenitors.

Treatment for the purposes described herein can be needed based on exposure to an intensive chemotherapy regimen including exposure to one or more of alkylating agents, Ara-C, azathioprine, carboplatin, cisplatin, chlorambucil, clofarabine, cyclophosphamide, ifosfamide, mechlorethamine, mercaptopurine, oxaliplatin, taxanes, and vinca alkaloids (e.g., vincristine, vinblastine, vinorelbine, and vindesine).

Treatment for the purposes described herein can also be needed based on exposure to a myeloablative regimen for HCT. In particular embodiments, compositions disclosed herein are administered to a subject at risk of depleted bone marrow, or at risk for depleted or limited blood cell levels. Administration can be for the purpose of a bone marrow transplant. Administration can also supplement a bone marrow transplant and can occur prior to and/or after a bone marrow transplant. In particular embodiments, the compositions can be used to treat relapsed pediatric acute lymphoblastic leukemia (ALL). Typically, cord blood transplant (CBT) is a standard of care for ALL when a suitably matched donor cannot be timely identified.

In the context of cancers, therapeutically effective amounts have an anti-cancer effect. An anti-cancer effect can be quantified by observing a decrease in the number of cancer cells, a decrease in the number of metastases, a decrease in tumor volume, an increase in life expectancy, induction of apoptosis of cancer cells, induction of cancer cell death, inhibition of cancer cell proliferation, inhibition of tumor growth, prevention of metastasis, prolongation of a subject's life, and/or reduction of relapse or re-occurrence of the cancer following treatment.

As indicated, administration of composition disclosed herein to a subject can occur at any time within a treatment regimen deemed helpful by an administering professional. As examples, compositions can be administered to a subject, e.g., before, at the same time, or after chemotherapy, radiation therapy or a bone marrow transplant.

Treatment for the purposes described herein can be needed based on exposure to acute ionizing radiation and/or exposure to other drugs that can cause bone marrow suppression or hematopoietic deficiencies including antibiotics, penicillin, ganciclovir, daunomycin, sulfa drugs, phenothiazines, tranquilizers, meprobamate, analgesics, aminopyrine, dipyrone, anticonvulsants, phenytoin, carbamazepine, antithyroids, propylthiouracil, methimazole, and diuretics. In particular embodiments, treatment can be needed due to treatment for renal disease or renal failure (e.g., dialysis). Immunodeficiencies may also be the result of other medical treatments.

In particular embodiments, the subject has blood loss due to, e.g., trauma, or is at risk for blood loss. In particular embodiments, the subject has depleted bone marrow related to, e.g., congenital, genetic or acquired syndrome characterized by bone marrow loss or depleted bone marrow. In particular embodiments, the subject is in need of hematopoiesis.

Examples of hematopoietic diseases and disorders that can be treated by administration of the disclosed ZTG-expanded HSC populations include:

I. Diseases resulting from a failure or dysfunction of normal blood cell production and maturation, such as hyperproliferative stem cell disorders, myelodysplastic syndrome, myelofibrosis (e.g., agnogenic myeloid metaplasia myelofibrosis), aplastic anemia, pancytopenia, agranulocytosis, thrombocytopenia, red cell aplasia, and Blackfan-Diamond syndrome due to drugs, radiation, or infection, and idiopathic disorders. Severe thrombocytopenia may result from genetic defects such as Fanconi's Anemia, Wiscott-Aldrich, or May-Hegglin syndromes. Acquired thrombocytopenia may result from auto- or allo-antibodies as in immune thrombocytopenia purpura, systemic lupus erythrematosus, hemolytic anemia, or fetal maternal incompatibility. In addition, splenomegaly, disseminated intravascular coagulation, thrombotic thrombocytopenic purpura, infection, and/or prosthetic heart valves may result in thrombocytopenia. Thrombocytopenia may also result from marrow invasion by carcinoma, lymphoma, leukemia, or fibrosis.

II. Hematopoietic malignancies, such as leukemia (e.g., acute lymphoblastic (lymphocytic) leukemia, chronic lymphocytic leukemia, acute myelogenous leukemia, or chronic myelogenous leukemia), acute malignant myelosclerosis, multiple myeloma, polycythemia vera, Waldenström macroglobulinemia, Hodgkin's lymphoma, and non-Hodgkin's lymphoma.

III. Immunosuppression in patients with malignant solid tumors, such as malignant melanoma, carcinoma of the stomach, ovarian carcinoma, breast carcinoma, small cell lung carcinoma, Retinoblastoma, testicular carcinoma, glioblastoma, rhabdomyosarcoma, neuroblastoma, Ewing's sarcoma, and lymphoma.

IV. Autoimmune diseases, such as rheumatoid arthritis, diabetes type I, chronic hepatitis, multiple sclerosis, and systemic lupus erythematosus.

V. Genetic (congenital) disorders, including Chediak-Higashi syndrome and a variety of anemias, such as familial aplastic anemia, Fanconi's syndrome, Bloom's syndrome, pure red cell aplasia (PRCA), dyskeratosis congenital, Blackfan-Diamond syndrome, congenital dyserythropoietic syndromes I-IV, Shwachman-Diamond syndrome, dihydrofolate reductase deficiencies, formamino transferase deficiency, Lesch-Nyhan syndrome, congenital spherocytosis, congenital elliptocytosis, congenital stomatocytosis, congenital Rh null disease, paroxysmal nocturnal hemoglobinuria, G6PD (glucose-6-phosphate dehydrogenase) variants 1, 2, 3, pyruvate kinase deficiency, congenital erythropoietin sensitivity deficiency, sickle cell disease and trait, thalassemia alpha, beta, and gamma, and methemoglobinemia.

VI. Infection with a pathogen (e.g., viral, bacterial, or fungal) that causes failure or dysfunction of normal blood cells. Examples of infections that cause blood cell dysfunction include HIVI, HIVII, HTLVI, HTLVII, and HTLVIII.

Compositions can be effective to provide engraftment when assayed at less than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 days; 1, 2, 3, 4, 5, 6, 7, 8, 9, 10 weeks (or more or less than 1, 2, 3, 4, 5, 6, 7, 8, 9, 10 weeks); 1; 2, 3, 4, 5, 6, 7, 8, 9, 10, 11, 12 months (or more or less than 1, 2, 3, 4, 5, 6, 7, 8, 9, 10, 11, 12 months); or 1, 2, 3, 4, 5 years (or more or less than 1, 2, 3, 4, 5 years) after administration of the composition to a subject. In particular embodiments, the composition is effective to provide engraftment when assayed within 10 days, 2 weeks, 3 weeks, 4 weeks, 6 weeks, or 13 weeks after administration of the composition to a subject.

As indicated previously, in particular embodiments, ZTG-expanded HSC populations can include cells with genetic modifications. In particular embodiments, a gene can be selected to provide a therapeutically effective response against a condition that, in particular embodiments, is inherited. In particular embodiments, the condition can be Grave's Disease, rheumatoid arthritis, pernicious anemia, Multiple Sclerosis (MS), inflammatory bowel disease, systemic lupus erythematosus (SLE), adenosine deaminase deficiency (ADA-SCID) or severe combined immunodeficiency disease (SCID), Wiskott-Aldrich syndrome (WAS), chronic granulomatous disease (CGD), Fanconi anemia (FA), Battens disease, adrenoleukodystrophy (ALD) or metachromatic leukodystrophy (MLD), muscular dystrophy, pulmonary aveolar proteinosis (PAP), pyruvate kinase deficiency, Shwachmann-Diamond-Blackfan anemia, dyskeratosis congenita, cystic fibrosis, Parkinson's disease, Alzheimer's disease, or amyotrophic lateral sclerosis (Lou Gehrig's disease). In particular embodiments, depending on the condition, the therapeutic gene may be a gene that encodes a protein and/or a gene whose function has been interrupted. Exemplary therapeutic gene and gene products include: soluble CD40; CTLA; Fas L; antibodies to CD4, CD5, CD7, CD52, etc.; antibodies to IL1, IL2, IL6; an antibody to TCR specifically present on autoreactive T cells; IL4; IL10; IL12; IL₁₃; IL1Ra, sIL1RI, sIL1RII; sTNFRI; sTNFRII; antibodies to TNF; P53, PTPN22, and DRB1*1501/DQB1*0602; globin family genes; WAS; phox; FANC family genes; dystrophin; pyruvate kinase; CLN3; ABCD1; arylsulfatase A; SFTPB; SFTPC; NLX2.1; ABCA3; GATA1; ribosomal protein genes; TERT; TERC; DKC1; TINF2; CFTR; LRRK2; PARK2; PARK7; PINK1; SNCA; PSEN1; PSEN2; APP; SOD1; TDP43; FUS; ubiquilin 2; and/or C9ORF72. Therapeutically effective amounts may provide function to immune and other blood cells and/or microglial cells or may alternatively—depending on the treated condition—inhibit lymphocyte activation, induce apoptosis in lymphocytes, eliminate various subsets of lymphocytes, inhibit T cell activation, eliminate or inhibit autoreactive T cells, inhibit Th-2 or Th-1 lymphocyte activity, antagonize IL1 or TNF, reduce inflammation, induce selective tolerance to an inciting agent, reduce or eliminate an immune-mediated condition; and/or reduce or eliminate a symptom of the immune-mediated condition. Therapeutically effective amounts may also provide functional DNA repair mechanisms; surfactant protein expression; telomere maintenance; lysosomal function; breakdown of lipids or other proteins such as amyloids; permit ribosomal function; and/or permit development of mature blood cell lineages which would otherwise not develop such as macrophages other white blood cell types.

As another example, a gene can be selected to provide a therapeutically effective response against diseases related to red blood cells and clotting. In particular embodiments, the disease is a hemoglobinopathy like thalassemia, or a sickle cell disease/trait. The therapeutic gene may be, for example, a gene that induces or increases production of hemoglobin; induces or increases production of beta-globin, or alpha-globin; or increases the availability of oxygen to cells in the body. The therapeutic gene may be, for example, HBB or CYB5R₃. Exemplary effective treatments may, for example, increase blood cell counts, improve blood cell function, or increase oxygenation of cells in patients. In another particular embodiment, the disease is hemophilia. The therapeutic gene may be, for example, a gene that increases the production of coagulation/clotting factor VIII or coagulation/clotting factor IX, causes the production of normal versions of coagulation factor VIII or coagulation factor IX, a gene that reduces the production of antibodies to coagulation/clotting factor VIII or coagulation/clotting factor IX, or a gene that causes the proper formation of blood clots. Exemplary therapeutic genes include F8 and F9. Exemplary effective treatments may, for example, increase or induce the production of coagulation/clotting factors VIII and IX; improve the functioning of coagulation/clotting factors VIII and IX, or reduce clotting time in subjects.

As another example, a gene can be selected to provide a therapeutically effective response against a lysosomal storage disorder. In particular embodiments, the lysosomal storage disorder is mucopolysaccharidosis (MPS), type I; MPS II or Hunter Syndrome; MPS III or Sanfilippo syndrome; MPS IV or Morquio syndrome; MPS V; MPS VI or Maroteaux-Lamy syndrome; MPS VII or sly syndrome; alpha-mannosidosis; beta-mannosidosis; glycogen storage disease type I, also known as GSDI, von Gierke disease, or Tay Sachs; Pompe disease; Gaucher disease; Fabry disease. The therapeutic gene may be, for example a gene encoding or inducing production of an enzyme, or that otherwise causes the degradation of mucopolysaccharides in lysosomes. Exemplary therapeutic genes include IDUA or iduronidase, IDS, GNS, HGSNAT, SGSH, NAGLU, GUSB, GALNS, GLB1, ARSB, and HYAL1. Exemplary effective genetic therapies for lysosomal storage disorders may, for example, encode or induce the production of enzymes responsible for the degradation of various substances in lysosomes; reduce, eliminate, prevent, or delay the swelling in various organs, including the head (exp. Macrocephaly), the liver, spleen, tongue, or vocal cords; reduce fluid in the brain; reduce heart valve abnormalities; prevent or dilate narrowing airways and prevent related upper respiratory conditions like infections and sleep apnea; reduce, eliminate, prevent, or delay the destruction of neurons, and/or the associated symptoms.

As another example, a gene can be selected to provide a therapeutically effective response against a hyperproliferative disease. In particular embodiments, the hyperproliferative disease is cancer. The therapeutic gene may be, for example, a tumor suppressor gene, a gene that induces apoptosis, a gene encoding an enzyme, a gene encoding an antibody, or a gene encoding a hormone. Exemplary therapeutic genes and gene products include 101F6, 123F2 (RASSF1), 53BP2, abl, ABLI, ADP, aFGF, APC, ApoAl, ApoAlV, ApoE, ATM, BAI-1, BDNF, Beta*(BLU), bFGF, BLC1, BLC6, BRCA1, BRCA2, CBFA1, CBL, C-CAM, CFTR, CNTF, COX-1, CSFIR, CTS-1, cytosine deaminase, DBCCR-1, DCC, Dp, DPC-4, E1A, E2F, EBRB2, erb, ERBA, ERBB, ETS1, ETS2, ETV6, Fab, FCC, FGF, FGR, FHIT, fms, FOX, FUS 1, FUS1, FYN, G-CSF, GDAIF, Gene 21 (NPRL2), Gene 26 (CACNA2D2), GM-CSF, GMF, gsp, HCR, HIC-1, HRAS, hst, IGF, IL-1, IL-2, IL-3, IL-4, IL-5, IL-6, IL-7, IL-8, IL-9, IL-10, IL-11 IL-12, ING1, interferon α, interferon β, interferon γ, IRF-1, JUN, KRAS, LCK, LUCA-1 (HYAL1), LUCA-2 (HYAL2), LYN, MADH4, MADR2, MCC, mda7, MDM2, MEN-I, MEN-II, MLL, MMAC1, MYB, MYC, MYCL1, MYCN, neu, NF-1, NF-2, NGF, NOEY1, NOEY2, NRAS, NT3, NT5, OVCA1, p16, p21, p27, p53, p57, p73, p300, PGS, PIM1, PL6, PML, PTEN, raf, Rap1A, ras, Rb, RB1, RET, rks-3, ScFv, scFV ras, SEM A3, SRC, TAL1, TCL3, TFPI, thrombospondin, thymidine kinase, TNF, TP53, trk, T-VEC, VEGF, VHL, WT1, WT-1, YES, and zac1. Exemplary effective genetic therapies may suppress or eliminate tumors, result in a decreased number of cancer cells, reduced tumor size, slow or eliminate tumor growth, or alleviate symptoms caused by tumors.

As another example, a gene can be selected to provide a therapeutically effective response against an infectious disease. In particular embodiments, the infectious disease is human immunodeficiency virus (HIV). The therapeutic gene may be, for example, a gene rendering immune cells resistant to HIV infection, or which enables immune cells to effectively neutralize the virus via immune reconstruction, polymorphisms of genes encoding proteins expressed by immune cells, genes advantageous for fighting infection that are not expressed in the patient, genes encoding an infectious agent, receptor or coreceptor; a gene encoding ligands for receptors or coreceptors; viral and cellular genes essential for viral replication including; a gene encoding ribozymes, antisense RNA, small interfering RNA (siRNA) or decoy RNA to block the actions of certain transcription factors; a gene encoding dominant negative viral proteins, intracellular antibodies, intrakines and suicide genes. Exemplary therapeutic genes and gene products include α2β1; ανβ3; ανβ5; ανβ63; BOB/GPR15; Bonzo/STRL-33/TYMSTR; CCR2; CCR3; CCR5; CCR8; CD4; CD46; CD55; CXCR4; aminopeptidase-N; HHV-7; ICAM; ICAM-1; PRR2/HveB; HveA; α-dystroglycan; LDLR/a2MR/LRP; PVR; PRR1/HveC; and laminin receptor. A therapeutically effective amount for the treatment of HIV, for example, may increase the immunity of a subject against HIV, ameliorate a symptom associated with AIDS or HIV, or induce an innate or adaptive immune response in a subject against HIV. An immune response against HIV may include antibody production and result in the prevention of AIDS and/or ameliorate a symptom of AIDS or HIV infection of the subject, or decrease or eliminate HIV infectivity and/or virulence.

As is understood by one of ordinary skill in the art, animal models of different blood disorders and cancers are well known and can be used to assess effectiveness of particular treatment paradigms, as necessary or beneficial.

The Examples and Exemplary Embodiments below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### (vii) Exemplary Embodiments

1. A method of expanding a CD34+ hematopoietic cell population, including:
incorporating the cell population (containing HSC) into an environment including a three-dimensional hydrogel for a period and under conditions that result in expansion, thereby expanding the CD34+ hematopoietic cell population.
2. A method of embodiment 1 wherein the hydrogel is an ultra-low fouling hydrogel.
3. A method of embodiment 1 or 2 wherein the hydrogel is a zwitterionic hydrogel (ZTG).
4. A method of any of embodiments 1-3, wherein at least of a portion of the CD34+ hematopoietic cells within the population are genetically-modified, for example to express a therapeutic gene and/or gene product listed in embodiment 140.
5. A method of any of embodiments 3-5, wherein the final expanded CD34+ hematopoietic cell population is a ZTG-expanded HSC population.
6. A method of any of embodiments 1-5, wherein the hydrogel has a kPa of at least 0.7.
7. A method of any of embodiments 1-5, wherein the hydrogel has a kPA of 0.7-5.
8. A method of any of embodiments 1-7, wherein the initial cell seeding density is 800,000-2 million cells/ml.
9. A method of any of embodiments 1-8, wherein the initial cell seeding density is 1.2 million cells/ml.
10. A method of any of embodiments 1-9, wherein the period is 6-20 days.
11. A method of any of embodiments 1-9, wherein the period includes a first period of 10-18 days.
12. A method of any of embodiments 1-9, wherein the period includes a first period of 10-18 days and a second period of 6-14 days.
13. A method of any of embodiments 1-9, wherein the period includes a first period of 14 days and a second period of 10 days.
14. A method of any of embodiments 1-13, wherein the environment includes at least one growth factor selected from human stem cell factor (SCF), FMS-like tyrosine kinase 3 ligand (FLT3), thrombopoietin (TPO), interleukin-6 (IL-6), and interleukin-3 (IL-3).
15. A method of any of embodiments 1-14, wherein the environment includes SCF, FLT3, TPO, IL-6, and IL-3.
16. A method of any of embodiments 1-15, wherein the final expanded CD34+ hematopoietic cell population has an increased proportion of HSC versus partially or fully differentiated cells, as compared to a CD34+ hematopoietic cell population expanded under a relevant control condition.
17. A method any of embodiments 3-16, wherein the percentage of HSC in the final expanded CD34+ hematopoietic cell population increased after the ZTG expansion.
18. A method of any of embodiments 1-17, wherein the final expanded CD34+ hematopoietic cell population shows: (i) at least a 10-fold increase in HSC having a CD34+, CD38, CD45RA-, CD49f+, CD90+ phenotype as compared to before the expansion; (ii) has at least 90% of cells that are CD34+ and Lin-; and/or (iii) has at least 70% of cells that are CD34+ and CD45RA− as compared to the CD34+ hematopoietic cell population before the incorporating.
19. A method of any of embodiments 1-18, wherein the final expanded CD34+ hematopoietic cell population has: reduced metabolic rates following expansion as compared to a CD34+ hematopoietic cell population expanded under a relevant control condition.
20. A method of embodiment 19, wherein the reduced metabolic rates are demonstrated through a reduction in (i) glucose consumption; (ii) lactate secretion; and/or (iii) amino acid metabolism.
21. A method of any of embodiments 1-20, wherein the expanding cell population has decreased production of reactive oxygen species (ROS), as compared to a CD34+ hematopoietic cell population expanding under a relevant control condition.
22. A method of any of embodiments 1-21, wherein the final expanded CD34+ hematopoietic cell population has decreased mitochondrial mass and/or mitochondrial membrane potential, as compared to a CD34+ hematopoietic cell population expanded under a relevant control condition.
23. A method of any of embodiments 1-22, wherein the incorporating results in at least 10-fold expansion, at least 50-fold expansion, at least 100-fold expansion, at least 500-fold expansion, at least 600-fold expansion, at least 700-fold expansion, at least 800-fold expansion, at least 900-fold expansion, or at least 1,000-fold expansion of HSC within the CD34+ hematopoietic cell population as compared to the CD34+ hematopoietic cell population before the incorporating.
24. A method of any of embodiments 1-23, wherein the hydrogel includes a zwitterionic polymer, polyethylene glycol, and/or a saccharide.
25. A method of embodiment 24, wherein the zwitterionic polymer includes a four-arm poly(carboxybetaine acrylamide) tetracyclooctyne.
26. A method of any of embodiments 1-25, wherein the hydrogel includes a poly(EK) crosslinker.
27. A method of embodiment 26, wherein the poly(EK) crosslinker includes a bis(azide) di-functionalized polypeptide.
28. A method of embodiment 27, wherein the bis(azide) di-functionalized polypeptide includes Azide-GG-(KE)20-GPQGIWGQ-(KE)20GG-Azide (SEQ ID NO: 1).
29. A method of any of embodiments 1-28, wherein the hydrogel is formed via a copper-free, strain-promoted azide-alkyne cycloaddition reaction between terminal difluorinated cyclooctyne and azide moieties.
30. A method of any of embodiments 1-29, further including: isolating the CD34+ hematopoietic cell population from umbilical cord blood, peripheral blood, mobilized peripheral blood, bone marrow, or induced pluripotent stem cells.
31. A method of any of embodiments 1-30, further including: degrading the hydrogel such that the expanded CD34+ hematopoietic cell population is not incorporated within the hydrogel; and harvesting expanded HSC from the expanded CD34+ hematopoietic cell population for a use.
32. A method of embodiment 31, wherein the degrading utilizes metalloproteinase.
33. A method of expanding a CD34+ hematopoietic cell population to create a CD34+ hematopoietic cell population with an increased proportion of HSC versus partially or fully differentiated cells, including: encapsulating a CD34+ hematopoietic cell population within a hydrogel including a zwitterionic polymer for a period of time and under conditions that result in expansion, thereby expanding the CD34+ hematopoietic cell population to create a CD34+ hematopoietic cell population with an increased proportion of HSC versus partially or fully differentiated cells, wherein the increased proportion is compared to the starting CD34+ hematopoietic cell population before expansion and/or a CD34+ hematopoietic cell population expanded under a relevant control condition.
34. A method of embodiment 33, wherein the percentage of HSC in the CD34+ hematopoietic cell population increases after the encapsulation for the period of time and under the conditions.
35. A method of embodiment 33 or 34, wherein the final expanded CD34+ hematopoietic cell population shows: (i) at least a 10-fold increase in HSC having a CD34+, CD38-, CD45RA-, CD49f+, CD90+ phenotype as compared to before the expansion as compared to the CD34+ hematopoietic cell population before the encapsulating; (ii) has at least 90% of cells that are CD34+ and Lin-; and/or (iii) has at least 70% of cells that are CD34+ and CD45RA-.
36. A method of any of embodiments 33-35, wherein the final expanded CD34+ hematopoietic cell population has: reduced metabolic rates following expansion as compared to a CD34+ hematopoietic cell population expanded under a relevant control condition.
37. A method of embodiment 36, wherein the reduced metabolic rates are demonstrated through a reduction in (i) glucose consumption; (ii) lactate secretion; and/or (iii) amino acid metabolism.
38. A method of any of embodiments 33-37, wherein the expanding CD34+ hematopoietic cell population has decreased production of ROS as compared to a CD34+ hematopoietic cell population expanded under a relevant control condition.
39. A method of any of embodiments 33-38, wherein the final expanded CD34+ hematopoietic cell population has decreased mitochondrial mass and/or mitochondrial membrane potential, as compared to a CD34+ hematopoietic cell population expanded under a relevant control condition.
40. A method of any of embodiments 33-39, wherein the encapsulation results in at least 10-fold expansion, at least 50-fold expansion, at least 100-fold expansion, at least 500-fold expansion, at least 600-fold expansion, at least 700-fold expansion, at least 800-fold expansion, at least 900-fold expansion or at least 1,000-fold expansion of the HSC within the CD34+ hematopoietic cell population as compared to the CD34+ hematopoietic cell population before the encapsulating.
41. A method of any of embodiments 33-40, wherein the zwitterionic polymer includes a four-arm poly(carboxybetaine acrylamide) tetracyclooctyne.
42. A method of any of embodiments 33-41, wherein the zwitterionic polymer includes a star-shaped polymer.
43. A method of method of any of embodiments 33-42, wherein the hydrogel includes a di-functionalized peptide.
44. A method of embodiment 43, wherein the di-functionalized peptide includes a bis(azide) di-functionalized polypeptide.
45. A method of embodiment 44, wherein the bis(azide) di-functionalized polypeptide includes Azide-GG-(KE)20-GPQGIWGQ-(KE)20GG-Azide (SEQ ID NO: 1).
46. A method of any of embodiments 33-45, wherein the CD34+ hematopoietic cell population has an increased proportion of HSC versus partially or fully differentiated cells following expansion as compared to expansion of a CD34+ hematopoietic cell population using a relevant control condition.
47. A method of any of embodiments 33-46, wherein the hydrogel has a kPa between 0.7-5.
48. A method of any of embodiments 33-47, wherein the hydrogel is formed by mixing the zwitterionic polymer, a di-functionalized peptide, and a media on a surface suitable for cell culturing.
49. A method of embodiment 48, wherein the surface suitable for cell culturing includes at least one glass slide.
50. A method of embodiment 49, wherein the at least one glass slide is treated with a polysiloxane.
51. A method of any of embodiments 33-50, wherein the encapsulating includes suspending the CD34+ hematopoietic cell population in expansion media including SCF, FLT3, TPO, IL-6, and/or IL-3.
52. A method of any of embodiments 33-51, wherein the encapsulating includes suspending the CD34+ hematopoietic cell population in expansion media including SCF, FLT3, TPO, IL-6, and IL-3.
53. A method of any of embodiments 33-52, further including:

degrading the hydrogel such that the final expanded CD34+ hematopoietic cell population is not encapsulated within the hydrogel; and harvesting expanded HSC from the expanded CD34+ hematopoietic cell population for a use.

54. A method of producing a ZT-expanded HSC population with reduced metabolism following expansion including expanding a CD34+ hematopoietic cell population in a hydrogel environment thereby reducing HSC metabolism following expansion as compared to a CD34+ hematopoietic cell population expanded under a relevant control condition.
55. A method of embodiment 54, wherein the hydrogel is an ultra-low fouling hydrogel.
56. A method of any of embodiments 54 or 55, wherein the hydrogel has a kPa of at least 0.7.
57. A method of any of embodiments 54-56, wherein the hydrogel has a kPA of 0.7-5.
58. A method of any of embodiments 54-57, wherein the initial cell seeding density is 800,000-2 million cells/ml.
59. A method of any of embodiments 54-57, wherein the initial cell seeding density is 1.2 million cells/ml.
60. A method of any of embodiments 54-57, wherein the period is 6-20 days.
61. A method of any of embodiments 54-57, wherein the period includes a first period of 10-18 days.
62. A method of any of embodiments 54-57, wherein the period includes a first period of 10-18 days and a second period of 6-14 days.
63. A method of any of embodiments 54-57, wherein the period includes a first period of 14 days and a second period of 10 days.
64. A method of any of embodiments 54-63, wherein the environment includes at least one growth factor selected from SCF, FLT3, TPO, IL-6, and IL-3.
65. A method of any of embodiments 54-64, wherein the environment includes SCF, FLT3, TPO, IL-6, and IL-3.
66. A method of any of embodiment 54-65, wherein the final ZT-expanded HSC population shows an increased proportion of HSC versus partially or fully differentiated cells, as compared to a CD34+ hematopoietic cell population expanded under a relevant control condition.
67. A method of any of embodiments 54-66, wherein the percentage of HSC in the ZT-expanded HSC population is increased over the starting CD34+ hematopoietic cell population.
68. A method of any of embodiments 54-67, wherein the ZT-expanded HSC population shows: (i) at least a 10-fold increase in HSC having a CD34+, CD38-, CD45RA-, CD49f+, CD90+ phenotype as compared to before the expansion; (ii) has at least 90% of cells that are CD34+ and Lin-; and/or (iii) has at least 70% of cells that are CD34+ and CD45RA.
69. A method of any of embodiments 54-68, wherein the reduced metabolism is demonstrated through a reduction in (i) glucose consumption; (ii) lactate secretion; and/or (iii) amino acid metabolism.
70. A method of any of embodiments 54-69, wherein the expanding CD34+ hematopoietic cell population has: decreased production of ROS as compared to a CD34+ hematopoietic cell population expanding under a relevant control condition.
71. A method of any of embodiments 54-70, wherein the ZT-expanded HSC population has decreased mitochondrial mass and/or mitochondrial membrane potential, as compared to a CD34+ hematopoietic cell population expanded under a relevant control condition.
72. A method of any of embodiments 54-71, wherein the ZT-expanded HSC population has at least 10-fold expansion, at least 50-fold expansion, at least 100-fold expansion, at least 500-fold expansion, at least 600-fold expansion, at least 700-fold expansion, at least 800-fold expansion, at least 900-fold expansion or at least 1,000-fold expansion as compared with the starting CD34+ hematopoietic cell population.
73. A method of any of embodiments 54-72, wherein the hydrogel includes a zwitterionic polymer, polyethylene glycol, and/or a saccharide.
74. A method of embodiment 73, wherein the zwitterionic polymer includes a four-arm poly(carboxybetaine acrylamide) tetracyclooctyne.
75. A method of any of embodiments 54-74, wherein the hydrogel includes a poly(EK) crosslinker.
76. A method of embodiment 75, wherein the poly(EK) crosslinker includes a bis(azide) di-functionalized polypeptide.
77. A method of embodiment 76, wherein the bis(azide) di-functionalized polypeptide includes Azide-GG-(KE)20-GPQGIWGQ-(KE)20GG-Azide (SEQ ID NO: 1).
78. A method of any of embodiments 54-77, wherein the hydrogel is formed via a copper-free, strain-promoted azide-alkyne cycloaddition reaction between terminal difluorinated cyclooctyne and azide moieties.
79. A method any of embodiments 54-78, further including: degrading the hydrogel such that the ZT-expanded HSC population is not incorporated within the hydrogel; and harvesting expanded HSC from the expanded ZT-expanded HSC population for a use.
80. A method of embodiment 79, wherein the degrading utilizes metalloproteinase.
81. A system for expanding a CD34+ hematopoietic cell population, including: a polymer; a cross-linker that when mixed with the polymer forms a hydrogel; and optionally, media including at least one of bovine serum albumin, human insulin, human transferrin, 2-mercaptoethanol, and/or Iscove's modified Dulbecco's medium.
82. A system of embodiment 81, wherein the polymer includes a zwitterionic polymer, a polyethylene glycol and/or a saccharide.
83. A system of embodiment 81 or 82, wherein the cross-linker includes a poly(EK) cross-linker.
84. A system of any of embodiments 81-83, further including a surface for culturing CD34+ hematopoietic cells.
85. A system of embodiment 84, wherein the surface for culturing CD34+ hematopoietic cells includes at least one glass slide treated with a polysiloxane.
86. A system of any of embodiments 81-85, wherein the zwitterionic polymer includes a four-arm poly(carboxybetaine acrylamide) tetracyclooctyne.
87. A system of any of embodiments 83-86, wherein the poly(EK) cross-linker includes a bis(azide) di-functionalized polypeptide.
88. A system of any of embodiments 81-87, further including at least one HSC encapsulated within the hydrogel following formation.
89. A system of any of embodiments 81-88, further including a frozen and/or a previously frozen CD34+ hematopoietic cell population.
90. A system of any of embodiments 81-89, wherein the hydrogel is a three-dimensional hydrogel following formation.
91. A system of any of embodiments 81-90 including: zwitterionic monomers and a cross-linker.
92. A system of embodiment 91, wherein the zwitterionic monomers have carboxybetaine group pendant groups.
93. A system of embodiment 92, wherein the carboxybetaine group pendant groups have the formula —Ra-N+(Rb)(Rc)-Rd-CO2-.
94. A system of embodiment 93, where Ra is a linker group that covalently couples a polymer backbone to the cationic nitrogen center of the carboxybetaine group.
95. A system of embodiment 93 or 94, wherein Rb is a nitrogen substituent.
96. A system of any of embodiments 93-95, wherein Rc is nitrogen substituent.
97. A system of any of embodiments 93-96, wherein Rd is a linker group that covalently couples a cationic nitrogen center to the carboxy group of a carboxybetaine group.
98. A system of any of embodiments 93-97, wherein the components of the system form a star shaped zwitterionic polymer.
99. A system of any of embodiments 93-98, further including a zwitterionic polymer.
100. A system of embodiment 99, wherein the further included zwitterionic polymer is a star-shaped zwitterionic polymer.
101. A system of any of embodiments 93-100, wherein the formed and/or included polymer includes repeating units having formula (I):

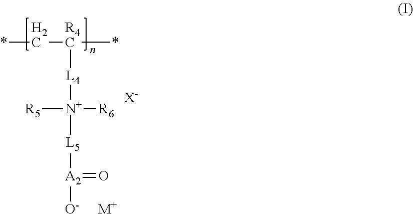

102. A system of embodiment 101, wherein R₄ is selected from hydrogen, fluorine, trifluoromethyl, C₁-C₆ alkyl, and C₆-C₁₂ aryl groups.
103. A system of embodiment 101, wherein R₄ is C₁-C₃ alkyl.
104. A system of any of embodiments 101-103, wherein R₅ and R₆ are independently selected from alkyl and aryl, or taken together with the nitrogen to which they are attached form a cationic center.
105. A system of any of embodiments 101-104, wherein R₅ and R₆ are C₁-C₃ alkyl.
106. A system of any of embodiments 101-105, wherein L₄ is a linker that covalently couples the cationic center [N₊(R⁵)(R₆)] to the polymer backbone [—(CH₂—CR₄)ₙ—].
107. A system of any of embodiments 101-106, wherein L₄ is selected from —C(O)O—(CH₂)ₙ— or —C(O)NH—(CH₂)ₙ—, wherein n is an integer from 1 to 20.
108. A system of any of embodiments 101-107, wherein L₄ is —C(O)O—(CH₂)ₙ—, and wherein n is 1-6.
109. A system of any of embodiments 101-108, wherein L₅ is a linker that covalently couples the anionic center [A₂(O)O⁻] to the cationic center.
110. A system of any of embodiments 101-109, wherein L₅ is —(CH₂)ₙ—, and where n is an integer from 1 to 20.
111. A system of any of embodiments 101-110, wherein A₂ is C, SO, SO₂, P, or P0.
112. A system of any of embodiments 101-111, wherein A₂ is C or SO.
113. A system of any of embodiments 101-112, wherein n is an integer from 5 to 10,000.
114. A system of any of embodiments 101-113, wherein n is an integer from 5 to 5,000.
115. A system of embodiment 101, wherein R₄, R₅, and R₆ are methyl, L₄ is —C(O)O—(CH₂)₂—, L₅ is —(CH₂)—, A₁ is C, and n is an integer from 10 to 1,000.
116. A system of any of embodiments 101-115, wherein * represents the point at which the repeating unit is covalently linked to an adjacent repeating unit or a functional group useful for forming hydrogels.
117. A system of any of embodiments 101-116, wherein pendant zwitterionic groups can be internal salts and M⁺ and X⁻ can be absent.
118. A system of any of embodiments 81-117, wherein the formed and/or included polymer includes poly(carboxybetaine methacrylate); poly(phosphobetaine methacrylate); poly(sulfobetaine methacrylate); and/or poly(carboxymethyl betaine).
119. A system of any of embodiments 81-118, wherein the cross-linker is a zwitterionic peptide.
120. A system of any of embodiments 81-119, wherein the cross-linker is degraded by a product released by an expanding CD34+ hematopoietic cell population.
121. A system of embodiment 87, wherein the bis(azide) di-functionalized polypeptide includes Azide-GG-(KE)20-GPQGIWGQ-(KE)20GG-Azide (SEQ ID NO: 1).
122. A system of any of embodiments 81-121, further including metalloproteinase.
123. A system of any of embodiments 81-122, further including SCF, FLT3, TPO, IL-6, and/or IL-3.
124. A system of any of embodiments 81-123, further including SCF, FLT3, TPO, IL-6, and/or IL-3.
125. A method of repopulating an immune system in a subject in need thereof including: administering a therapeutically effective amount of composition including a ZTG-expanded HSC population to the subject, thereby repopulating the immune system of the subject.
126. A method of embodiment 125 wherein the repopulating provides long-term hematopoietic reconstitution.
127. A method of embodiment 125 or 126 wherein the subject is a human subject.
128. A method of any of embodiments 125-127 wherein at least a portion of cells within the ZTG-expanded HSC population are genetically modified.
129. A method of embodiment 128 wherein the genetic modification results in expression of a therapeutic gene and/or gene product.
130. A method of any of embodiments 125-129, wherein the therapeutically effective amount provides a prophylactic treatment and/or a therapeutic treatment.
131. A method of any of embodiments 125-130, wherein the subject is in need thereof due to exposure to one or more of an alkylating agent, Ara-C, azathioprine, carboplatin, cisplatin, chlorambucil, clofarabine, cyclophosphamide, ifosfamide, mechlorethamine, mercaptopurine, oxaliplatin, taxanes, vincristine, vinblastine, vinorelbine, and/or vindesine.
132. A method of any of embodiments 125-131, wherein the subject is in need thereof due to exposure to a myeloablative regimen for hematopoietic cell transplantation.
133. A method of any of embodiments 125-132, wherein the subject is in need thereof due to exposure to acute ionizing radiation and/or exposure to drugs that cause bone marrow suppression and/or hematopoietic deficiencies including at least one of an antibiotic, penicillin, ganciclovir, daunomycin, a sulfa drug, a phenothiazine, a tranquilizer, meprobamate, an analgesic, aminopyrine, dipyrone, an anticonvulsant, phenytoin, carbamazepine, an antithyroid, propylthiouracil, methimazole, and/or a diuretic.
134. A method of any of embodiments 125-133, wherein the subject is in need thereof due to a viral infection, a microbial infection, and/or a parasitic infection as a result of treatment for renal disease and/or renal failure.
135. A method of any of embodiments 125-134, wherein the subject is in need thereof due to an immunodeficiency in at least one of T and/or B lymphocytes, and/or rheumatoid arthritis.
136. A method of any of embodiments 125-135, wherein the subject is in need thereof due to at least one of aplastic anemia, Chediak-Higashi syndrome, systemic lupus erythematosus (SLE), leukemia, myelodysplastic syndrome, myelofibrosis, and/or thrombocytopenia.
137. A method of any of embodiments 125-136, wherein the subject is in need thereof due to trauma-related blood loss.
138. A method of any of embodiments 125-137, wherein the subject receives at least a portion of the expanded HSC population before, at the same time, and/or after chemotherapy, radiation therapy, and/or a bone marrow transplant.
139. A method of any of embodiments 125-138, wherein the subject is in need thereof due to an immunodeficiency, a pancytopenia, a neutropenia, and/or a leukopenia.
140. A method of any of embodiments 129-139, wherein the genetic modification leads to expression of a therapeutic gene and/or gene product including soluble CD40; CTLA; Fas L; antibodies to CD4, CD5, CD7, CD52; antibodies to IL1, IL2, IL6; an antibody to TCR specifically present on autoreactive T cells; IL4; IL10; IL12; IL13; IL1Ra, sIL1RI, sIL1RII; sTNFRI; sTNFRII; antibodies to TNF; P53, PTPN22, and DRB1*1501/DQB1*0602; globin family genes; WAS; phox; FANC family genes; dystrophin; pyruvate kinase; CLN3; ABCD1; arylsulfatase A; SFTPB; SFTPC; NLX2.1; ABCA3; GATA1; ribosomal protein genes; TERT; TERC; DKC1; TINF2; CFTR; LRRK2; PARK2; PARK7; PINK1; SNCA; PSEN1; PSEN2; APP; SOD1; TDP43; FUS; ubiquilin 2; C9ORF72, IDUA or iduronidase, IDS, GNS, HGSNAT, SGSH, NAGLU, GUSB, GALNS, GLB1, ARSB, and HYAL1, 101F6, 123F2 (RASSF1), 53BP2, abl, ABLI, ADP, aFGF, APC, ApoAl, ApoAlV, ApoE, ATM, BAI-1, BDNF, Beta*(BLU), bFGF, BLC1, BLC6, BRCA1, BRCA2, CBFA1, CBL, C-CAM, CFTR, CNTF, COX-1, CSFIR, CTS-1, cytosine deaminase, DBCCR-1, DCC, Dp, DPC-4, E1A, E2F, EBRB2, erb, ERBA, ERBB, ETS1, ETS2, ETV6, Fab, FCC, FGF, FGR, FHIT, fms, FOX, FUS 1, FUS1, FYN, G-CSF, GDAIF, Gene 21 (NPRL2), Gene 26 (CACNA2D2), GM-CSF, GMF, gsp, HCR, HIC-1, HRAS, hst, IGF, IL-1, IL-2, IL-3, IL-4, IL-5, IL-6, IL-7, IL-8, IL-9, IL-10, IL-11 IL-12, ING1, interferon α, interferon β, interferon γ, IRF-1, JUN, KRAS, LCK, LUCA-1 (HYAL1), LUCA-2 (HYAL2), LYN, MADH4, MADR2, MCC, mda7, MDM2, MEN-I, MEN-II, MLL, MMAC1, MYB, MYC, MYCL1, MYCN, neu, NF-1, NF-2, NGF, NOEY1, NOEY2, NRAS, NT3, NT5, OVCA1, p16, p21, p27, p53, p57, p73, p300, PGS, PIM1, PL6, PML, PTEN, raf, Rap1A, ras, Rb, RB1, RET, rks-3, ScFv, scFV ras, SEM A3, SRC, TAL1, TCL3, TFPI, thrombospondin, thymidine kinase, TNF, TP53, trk, T-VEC, VEGF, VHL, WT1, WT-1, YES, zac1, α2β1; ανβ3; ανβ5; ανβ63; BOB/GPR15; Bonzo/STRL-33/TYMSTR; CCR2; CCR3; CCR5; CCR8; CD4; CD46; CD55; CXCR4; aminopeptidase-N; HHV-7; ICAM; ICAM-1; PRR2/HveB; HveA; α-dystroglycan; LDLR/a2MR/LRP; PVR; PRR1/HveC; and laminin receptor.
141. A method of any of embodiments 129-140, further including expanding a CD34+ hematopoietic cell population to create a ZT-expanded HSC population according to a method of any of embodiments 1-80.
142. A method of creating a ZTG-expanded HSC population including using a system of embodiments 81-124 to practice a method of embodiments 1-80 or 141.
143. A method of any of embodiments 1-80, 141, or 142, wherein following the expansion the expanded HSC population has a higher proportion of HSC in a quiescent state as compared to a control CD34+ hematopoietic cell population expanded in a relevant control condition comprising expansion within a hydrophobic polystyrene flask or with a Notch agonist substrate.
144. A method of embodiment 143, wherein the higher proportion of HSC in a quiescent state is demonstrated through a reduced proportion of G₁ cells and an increased proportion of G₀ cells.
145. A hydrogel-expanded HSC population formed according to a method and/or including a feature of any of embodiments 1-80 1-80, 141, or 142.
146. A hydrogel-expanded HSC population of embodiment 145, formed using a system of embodiments 81-124.
147. A ZTG-expanded HSC population formed according to a method and/or including a feature of any of embodiments 1-80 1-80, 141, or 142.
148. A ZTG-expanded HSC population of embodiment 147, formed using a system of embodiments 81-124.
149. A ZTG-expanded HSC population of embodiment 147, wherein the feature includes a lower metabolic rate as compared to a control CD34+ hematopoietic cell population expanded in a relevant control condition comprising expansion within a hydrophobic polystyrene flask or with a Notch agonist substrate.
150. A ZTG-expanded HSC population of embodiment 149, wherein the lower metabolic rate is demonstrated through a reduction in one or more of (i) glucose consumption; (ii) lactate secretion; and (iii) amino acid metabolism.
151. A ZTG-expanded HSC population of embodiment 149 or 150, wherein the lower metabolic rate is demonstrated through a reduction in mitochondrial mass and/or mitochondrial membrane potential.
152. A ZTG-expanded HSC population of any of embodiments 147-151, wherein the feature includes a higher proportion of HSC in a quiescent state as compared to a control CD34+ hematopoietic cell population expanded in a relevant control condition comprising expansion within a hydrophobic polystyrene flask or with a Notch agonist substrate.
153. A ZTG-expanded HSC population of embodiment 152, wherein the higher proportion of HSC in a quiescent state is demonstrated through a reduced proportion of G₁ cells and an increased proportion of G₀ cells.
154. A ZTG-expanded HSC population of any of embodiments 147-153, wherein at least a portion of cells within the ZTG-expanded HSC population are genetically modified.
155. A ZTG-expanded HSC population of any of embodiments 147-154, wherein the genetic modification includes a modification of embodiment 140.
156. A composition comprising a ZTG-expanded HSC population of any of embodiments 147-155.
157. A composition comprising a therapeutically effective amount of a ZTG-expanded HSC population of any of embodiments 147-155.

### (viii) Experimental Examples

Like many primary cells and cell lines, HSPCs are commonly cultured in hydrophobic polystyrene flasks, exposing them to surroundings that differ greatly from their in vivo niche, a three-dimensional (3D) environment dominated by hydrophilic and zwitterionic cell membrane lipids (Bretscher, Nature, 258:43 (1975)). Hydrophobic materials provide abundant nonspecific interactions to all cell ligands (Steele, Biomaterials, 16:1057 (1995)), and, without being bound by theory, it is hypothesized that this continuous nonspecific stimulation of cells plays a key role in triggering HSPC differentiation in vitro. To explore this hypothesis, the in vivo niche was more closely modeled by culturing HSPC inside a ZTG. Zwitterionic polymers and peptides are super-hydrophilic and uniquely resistant to nonspecific interactions, and polyzwitterionic surfaces can substantially reduce and even completely eliminate protein attachment in complex physiological fluids including undiluted plasma and serum (Jiang, Adv Mater, 22:920 (2010)). In contrast to hydrophobic and amphiphilic materials, zwitterionic materials have little or even no effect on the activity of nearby or conjugated proteins (Keefe, Nat Chem, 4:59 (2012)), are able to resist collagenous capsule formation when implanted in mice (Sinclair et al., Biomacromolecules, 14:1587 (2013)), and circumvent antibody production during bloodstream circulation (Zhang et al., PNAS, 112:12046 (2015)).

Both cord blood (CB) and bone marrow (BM)-derived HSPC were encapsulated and cultured inside zwitterionic poly(carboxybetaine)-based hydrogels using a metalloproteinase-cleavable zwitterionic peptide to reversibly crosslink the gels (DeForest, Nat Mater 8:659 (2009); Anderson, Biomaterials, 32:3564 (2011); West & Hubbell, Macromolecules, 32:241 (1999). All components were designed to substantially reduce or even completely avoid nonspecific interactions with the encapsulated HSPCs, and the degradable crosslinks facilitated gradual growth and eventual cell recovery.

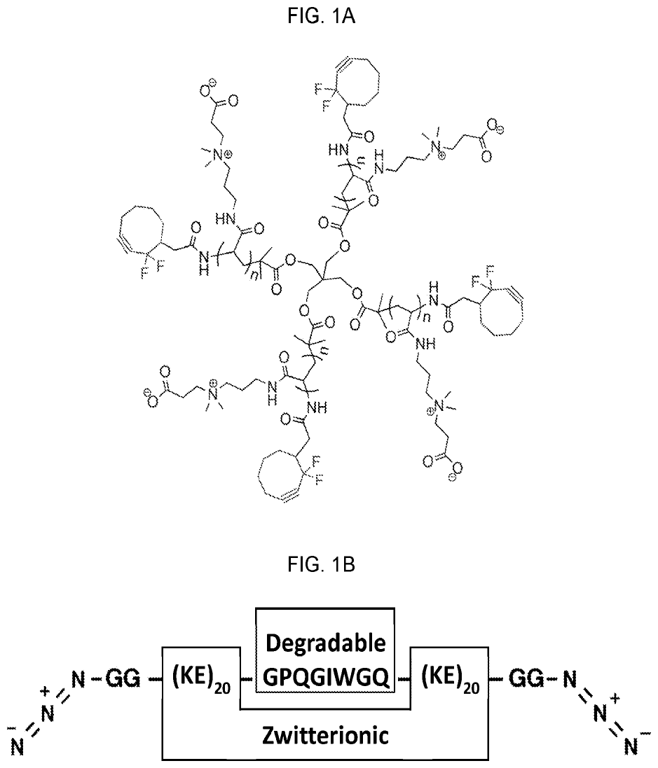

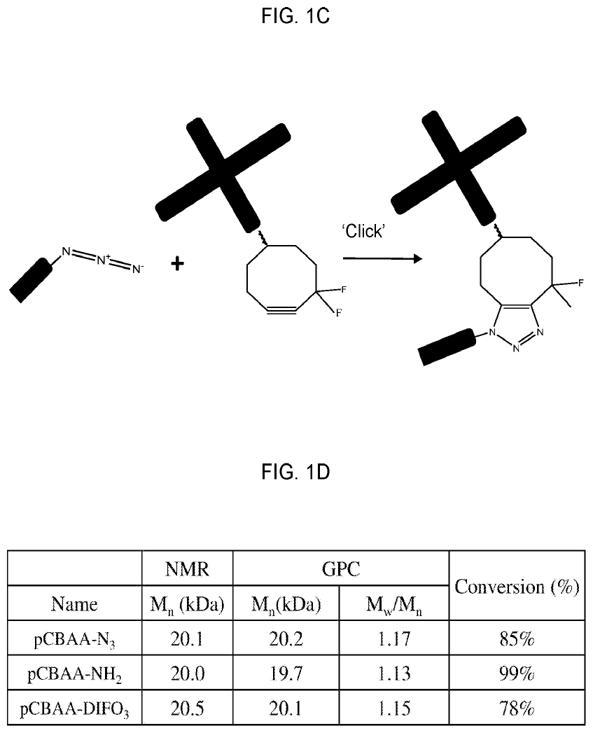

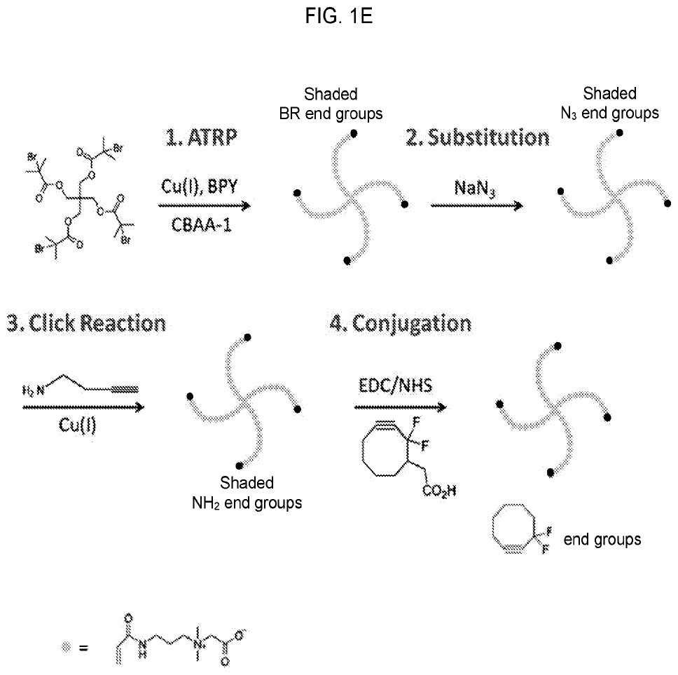

To enable gentle cell encapsulation in situ, a bioorthogonal and cytocompatible strain-promoted azide-alkyne cycloaddition (SPAAC) “click” reaction was used to form the ZTG. Four-arm poly(carboxybetaine acrylamide) (pCBAA) was terminated with difluorinated cyclooctynes (DIFO₃) and reacted with a bis(azide)-functionalized polypeptide (Azide-GG-(KE)₂₀-GPQGIWGQ-(KE)₂₀-GG-Azide) (SEQ ID NO: 1) to form an idealized three-dimensional network with minimal local defects (FIG. 1A-1E) (DeForest, Nat Mater, 8:659 (2009), DeForest & Anseth, Nat Chem, 3:925 (2011)). To maximize the cross-linker hydrophilicity, zwitterionic segments of twenty residues, alternating lysine (K) and glutamic acid (E), were added on each side of the metalloproteinase-cleavable motif previously reported (West & Hubbell, Macromolecules, 32:241 (1999); DeForest & Anseth, Nat Chem, 3:925 (2011)). Particularly in contrast to hydrophobic polystyrene (the most common material used to make cell culture flasks), nonspecific interactions between HSPC lysate or media proteins and either the pCBAA polymer or KE-dominated peptide (FIG. 2) were not detected. This finding suggests that these ZTG materials can significantly reduce and even completely eliminate nonspecific interactions in the culture platform, creating a “blank slate” for 3D stem cell culture.

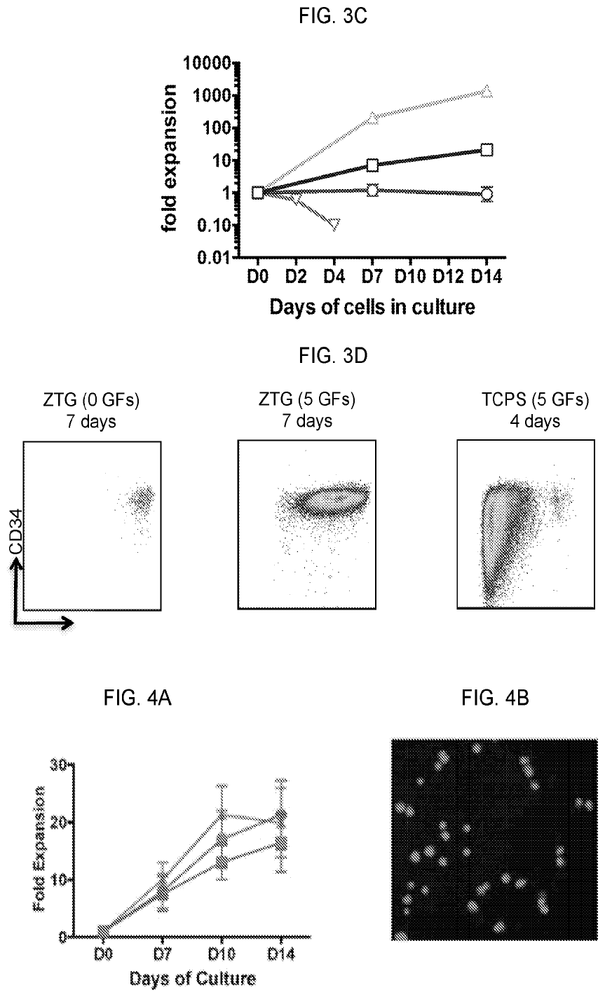

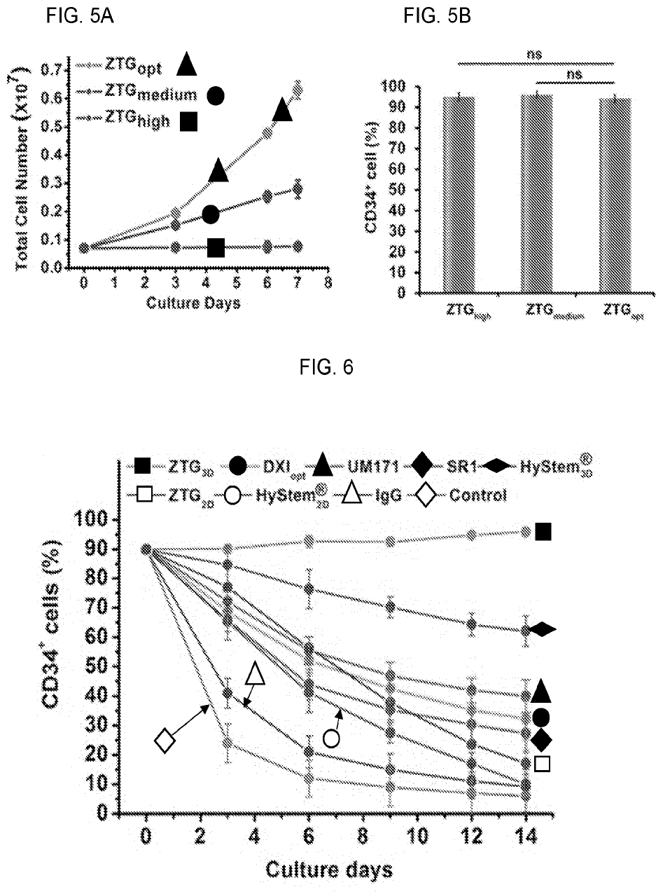

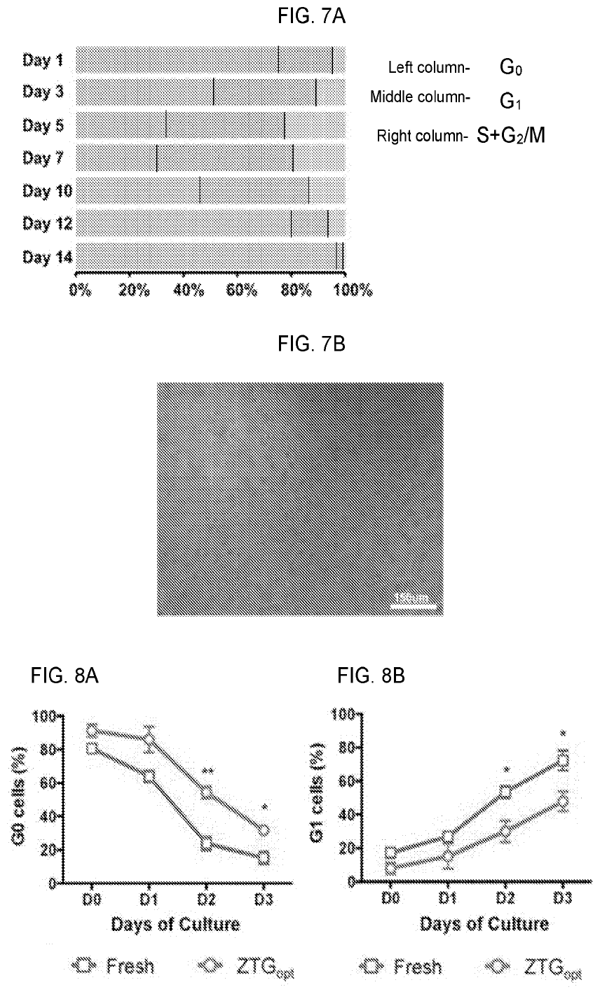

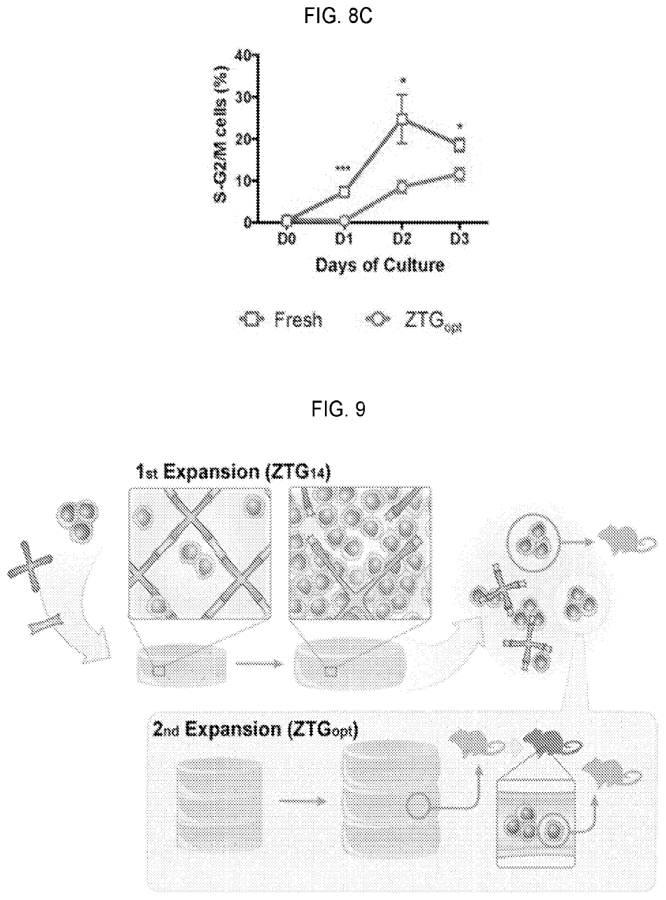

Fresh human CD34⁺ HSPC were isolated from CB. This purified cell population was encapsulated within the aforementioned ZTG in previously characterized growth factors and media (Csaszar et al., Cell Stem Cell, 10:218 (2012), Delaney et al., Nat Med, 16:232 (2010)), and proliferation responses were monitored. Without being bound by theory, HSPCs secrete small amounts of metalloproteinase, which gradually cleaves some of the peptide crosslinks during proliferation, allowing the hydrogel to relax and swell, which permits accommodation of the growing population. After an initial 14-day expansion period, exogenous metalloproteinase was added to fully disassemble the constructs and free the expanded cells. As presented in FIGS. 3A-3D, 4A, 4B, the ZTG condition can promote HSC survival and self-renewal under different culture conditions. The influence of the mechanical property of hydrogels on the expansion of HSC in ZTG culture is also examined to compare with those results from Holst et al., Nat Biotechnol, 28:1123 (2010) (FIGS. 5A, 5B). In this work, several state-of-the-art methods (including UM171, SR1, and Delta1 along with a commercial hyaluronic acid and polyethylene glycol-based HYSTEM® hydrogel designed to reduce cell attachment), were used as control groups. The isolated cells after each culture were immunophenotyped and evaluated for differentiation (defined as loss of CD34 expression). As presented in FIG. 6, when HSPCs were cultured on a hydrophobic tissue culture polystyrene (TOPS) substrate (control), a sharp decrease in CD34 expression was observed. While aforementioned UM171, SR1 and Delta1 culture can maintain CD34 expression at a much higher level than TOPS, some degree of differentiation was still apparent. Hydrophilic 2D substrates such as IgG protein coatings, and HYSTEM® also showed a mixture of CD34+ and CD34-expression. Importantly, the ZTG culture showed a striking effect in preserving CD34 expression during this 14-day culture. Among all the experimental groups, only the ZTG showed a surprisingly high frequency of CD34⁺ cells in the final harvested cell population (95.9%±3%). This encouraged pursuit of further expansion using ZTG culture. The cell cycle status of cells in the ZTG culture condition was investigated by staining with anti-Ki-67 and Hoechst 33342. As presented in FIGS. 7A, 7B, the cells began to exhibit G₁ and S+G₂/M phases in the first few days of ZTG culture. Then, cells in the G₁ and S+G₂/M phases gradually decreased and almost all cells converged into the G₀ phase by Day 14. In addition, when cells were transferred to regular cell culture flasks, both fresh cells and ZTG-expanded cells were able to enter into cell cycle (FIGS. 8A-8C).

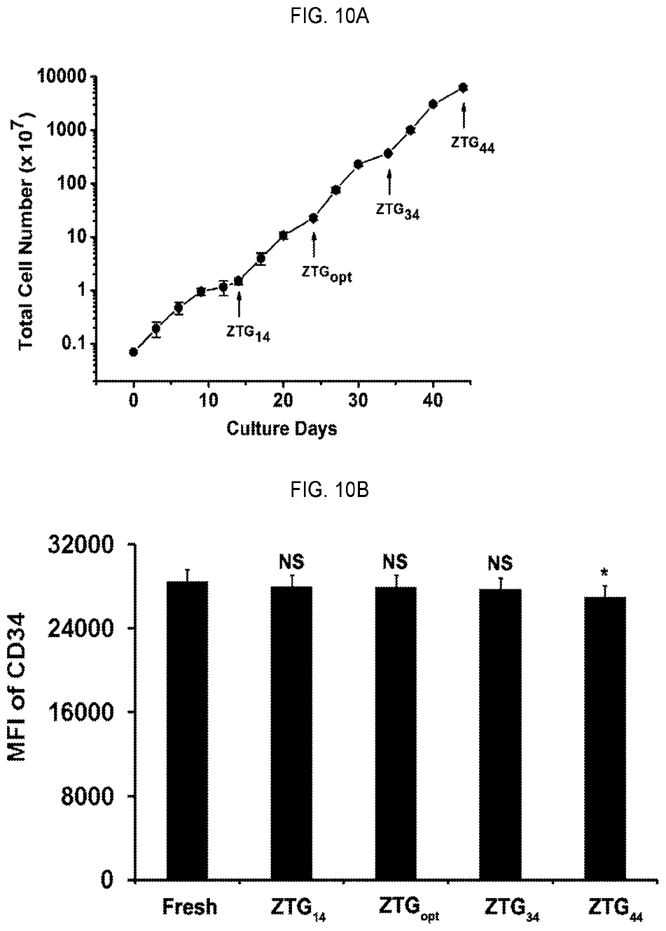

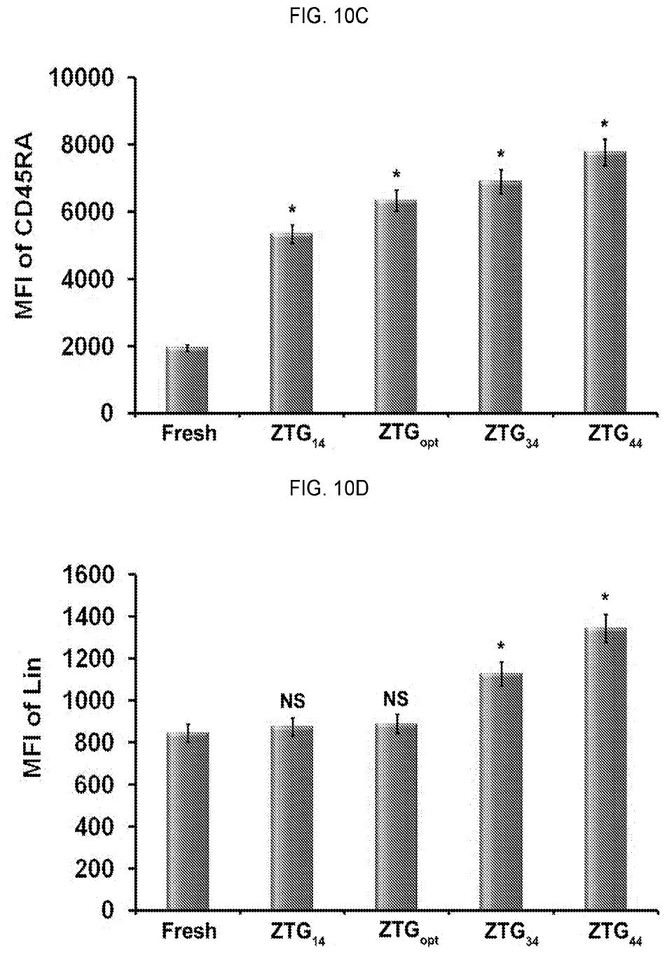

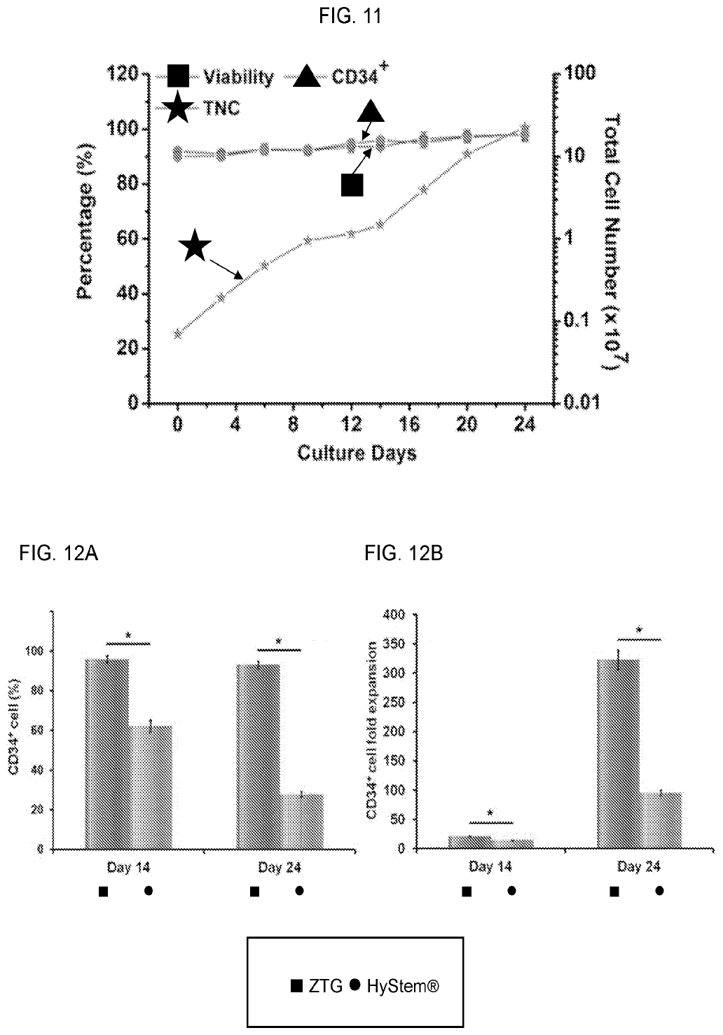

As presented in FIG. 9, after the initial 14-day expansion period, expanded cells were passaged into a new ZTG at an optimal seeding density for another 10 days of culture; a significant proliferation rate decrease was observed after this 10-day culture (FIG. 10A). In this work, the 24-day protocol was used as the optimized condition for CD34⁺ HSPC culture in the ZTG (ZTGₒₚₜ), in which expanded cells showed a higher cell number increase and minimal phenotype changes (FIGS. 10B-10D). After this, the final cell population was harvested for further evaluation and functional HSC assays (FIG. 9, bottom). Over this 24-day culture period, a 322-fold expansion of total nucleated cells (TNC) with excellent viability (94.2%±3%) and a surprisingly high frequency of CD34+ cells in the final harvested cell population (94.6%±2%) (FIG. 11) was achieved. In contrast, additional 10-day culture in HYSTEM® culture significantly decreased the CD34⁺ population and resulted in low expansion rate of CD34+ cells (FIGS. 12A, 12B). Cells harvested after the second 10-day culture period in the ZTG were more robust in terms of in vitro colony-forming unit (CFU) assays and in vivo engraftment, compared to cells cultured in the ZTG for only 14 days (FIGS. 13A, 13B).

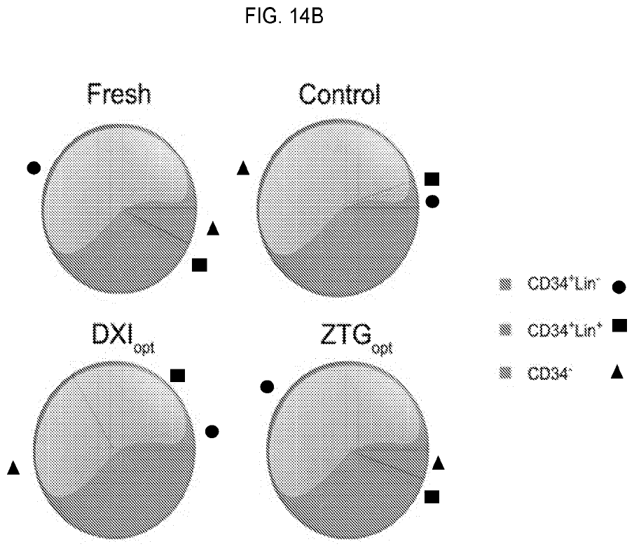

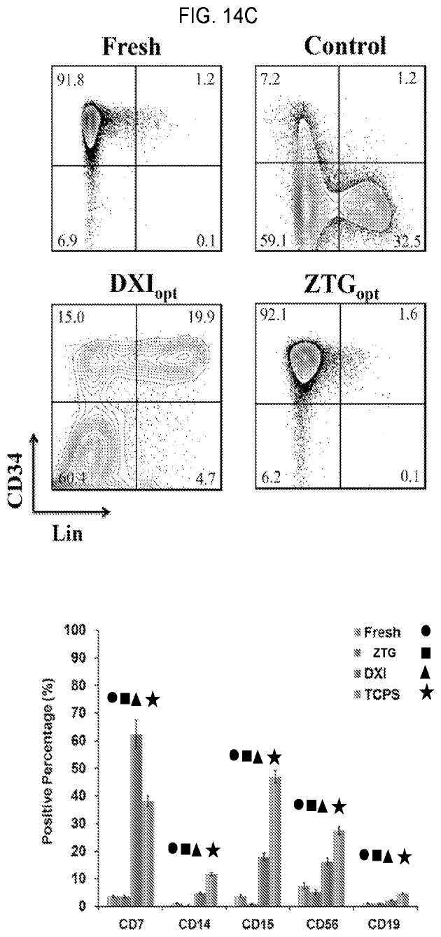

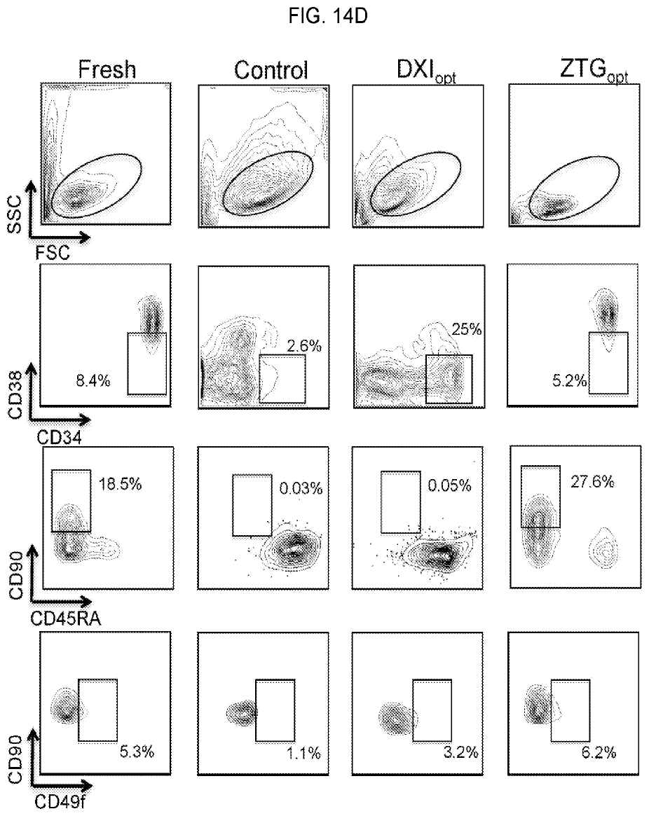

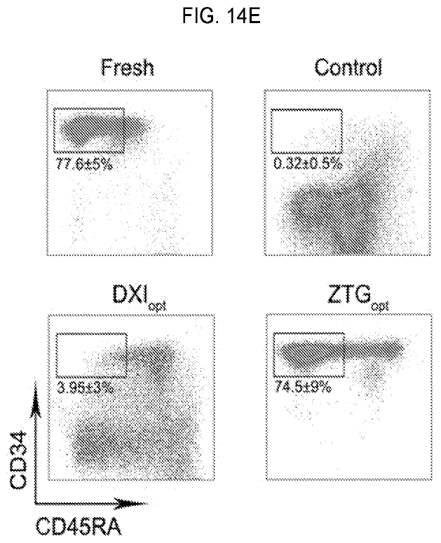

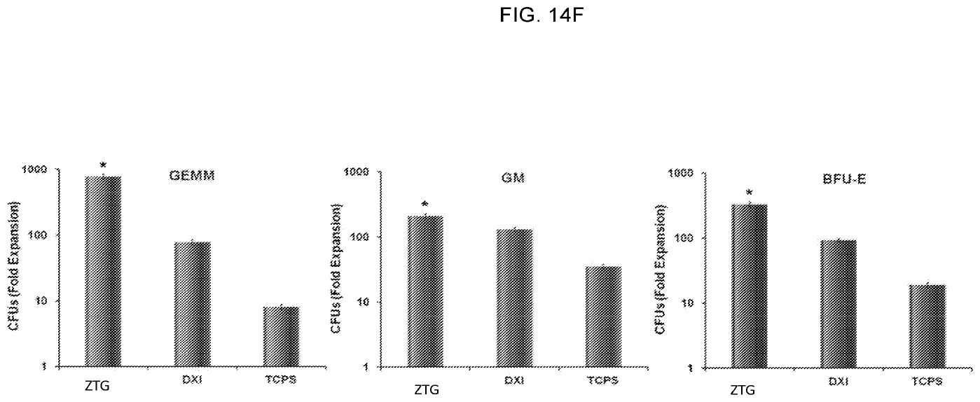

Next, the ZTGₒₚₜ expansion methodology was compared with a culture system using an engineered Notch ligand, an optimized Delta1^{ext-IgG} (DXI₀₀) and the culture of HSPC in unmodified flasks (control). Highly purified fresh CB CD34+ cells (93%±2% CD34+) were used to initiate cultures in all conditions. These cells were homogeneously small and round with scant and agranular cytoplasm and had round eccentric nuclei (FIG. 14A). The homogeneity of this population was retained after culture in the ZTGₒₚₜ, while both DXIₒₚₜ and control conditions resulted in heterogeneous populations of large cells with granular cytoplasm and irregular nuclei, indicating some degree of cell differentiation (FIG. 14A). FIG. 14A (bottom panel) shows the diameter of cells cultured in each condition. ZTGₒₚₜ culture conditions maintained a high percentage of CD34+ cells throughout the culture period with 93.7%±2% of the cultured cells expressing CD34. In comparison, 34.9%±2% of the DXIₒₚₜ expanded cells and only 8.4%±4% of the control culture remained CD34+(FIGS. 14B, 14C). More importantly, even though a significant portion of DXIₒₚₜ expanded cells continued to express CD34, only 15.0%±3% of these cells were also negative for lineage markers (CD7, CD14, CD15, CD19, and CD56), a two-fold increase when compared to the control condition result (7.2%±3%) (FIG. 14C).

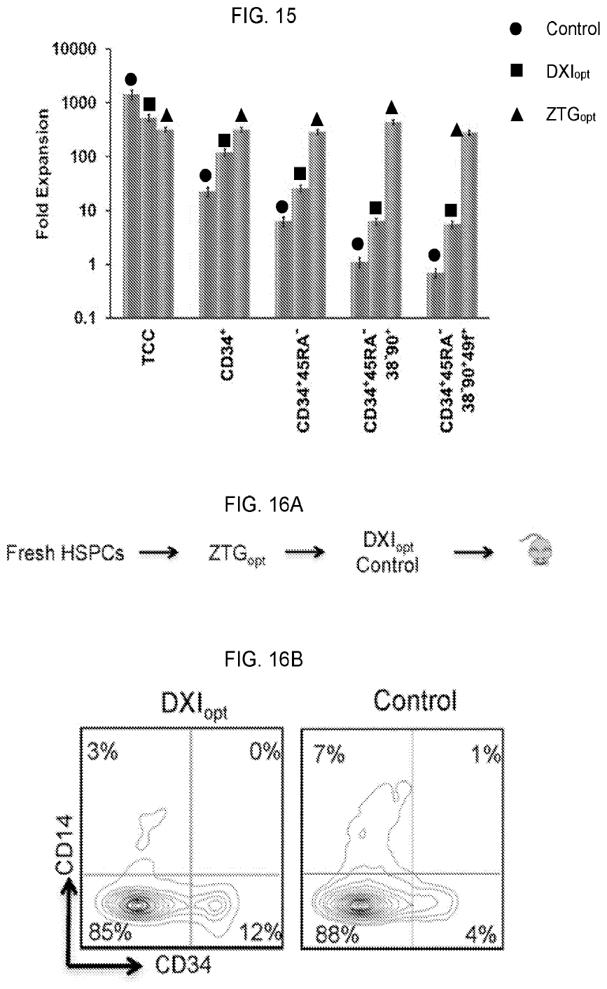

In stark contrast, 92.1%±1% of ZTGₒₚₜ cells were both CD34⁺ and Lin⁻, again showing no significant difference from fresh HSPC (91.8%±1%) (FIG. 14C). ZTGₒₚₜ cells maintained a phenotype very similar to fresh HSPCs (FIG. 14D), as well as a high frequency of CD34⁺CD45RA⁻ cells at 74.5%±9% of the total final cell population, compared to 77.6%±5% of the initial purified starting population (FIG. 14E). This is in contrast to the reduced CD34⁺CD45RA⁻ frequencies observed after culture in the DXIₒₚₜ (3.9%±3%) and control (0.3%±0.5%) cultures (FIG. 14E). The fold expansions, both total cell count (TCC) and primitive HSPC subsets, are shown in FIG. 15. Higher overall TCC proliferation was seen in the DXIₒₚₜ (530-fold) and control (1,450-fold) conditions, but a substantial fraction of these cells were negative for CD34 at the end of the cultures. In contrast, ZTGₒₚₜ expansion produced a final 322-fold TCC increase that was matched by a 319-fold increase in CD34+ cells and 284-fold increase in the most phenotypically primitive subset (CD34+CD38⁻CD45RA⁻CD49f⁺CD90⁺) (FIG. 15). These results are remarkable when compared with the increase of the same populations in DXIₒₚₜ (122-fold increase in CD34+ and 6-fold increase in CD34+CD38⁻CD45RA⁻CD49f⁺CD90⁺ cells) and control conditions (22-fold increase in CD34+ and 30% decrease in CD34⁺CD38⁻CD45RA⁻CD49f⁺CD90⁺ cells) (FIG. 15).

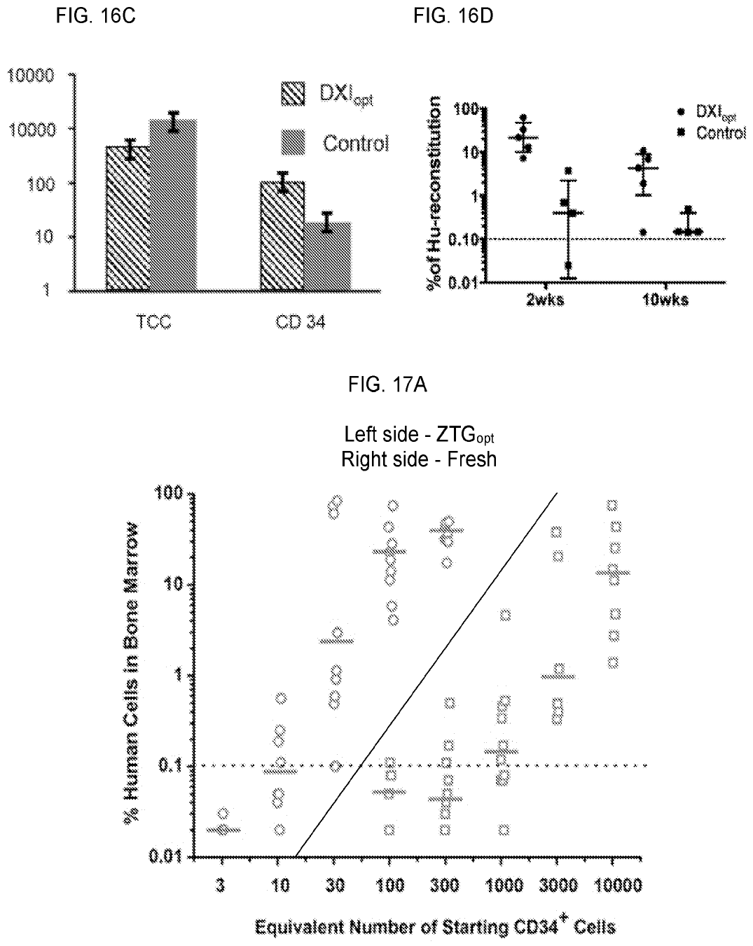

The negligible expansion of CD34⁻ cells (0.3-fold increase) and low expression of differentiation markers in the ZTGₒₚₜ-expanded population suggests the ZTG is particularly capable of enhancing HSC expansion while minimizing differentiation. To investigate whether the 24-day ZTGₒₚₜ cells behaved similarly to freshly isolated (non-cultured) HSPC in the DXIₒₚₜ system, cultures were initiated as per the DXIₒₚₜ methods using cells harvested after ZTGₒₚₜ culture (FIG. 16A). These 24-day ZTGₒₚₜ cells continued to retain their ex vivo expansion capacity signaled by Delta1^{ext-IgG} stimulation, as expected in this system (FIGS. 16B and 16C). As with freshly isolated HSPCs, ZTGₒₚₜ-cultured cells subsequently expanded in DXIₒₚₜ conditions rapidly reconstituted in NSG recipients, matching experiments using freshly isolated HSPCs from CB for Delta1^{ext-IgG} expansion (FIG. 16D) (Delaney et al., Nat Med, 16:232 (2010)).

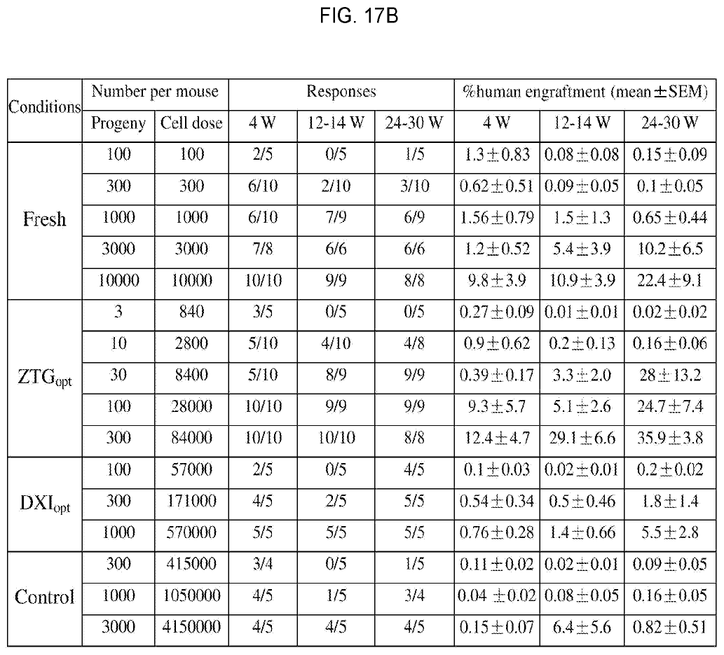

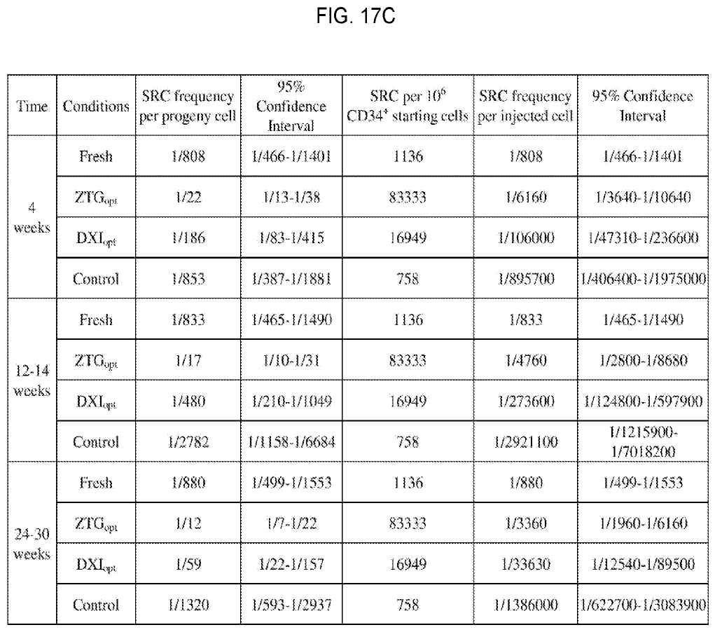

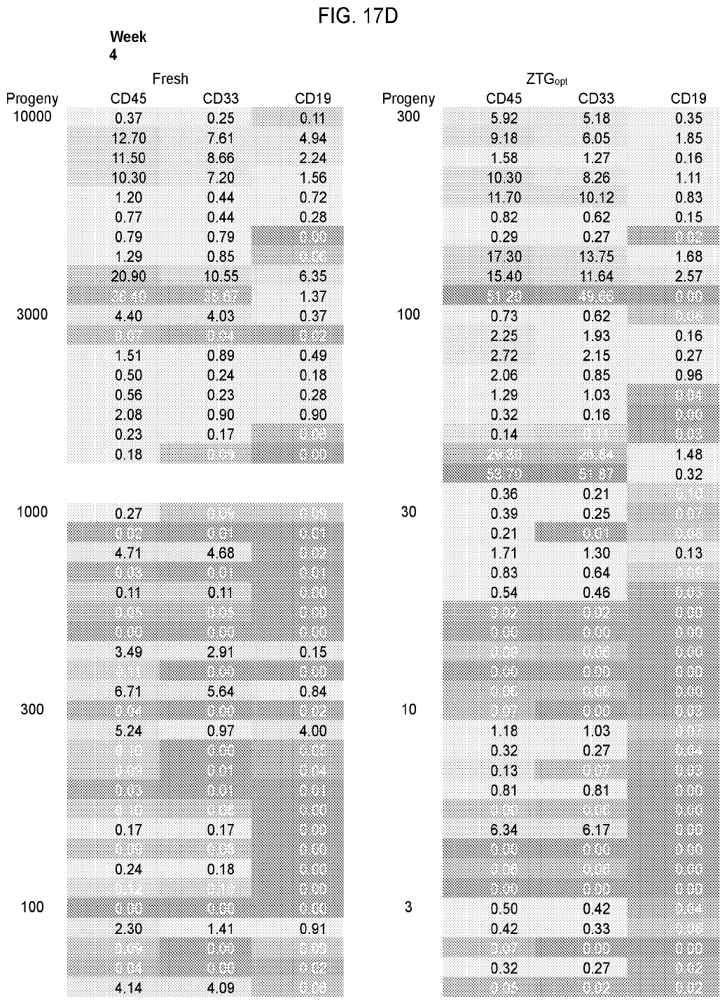

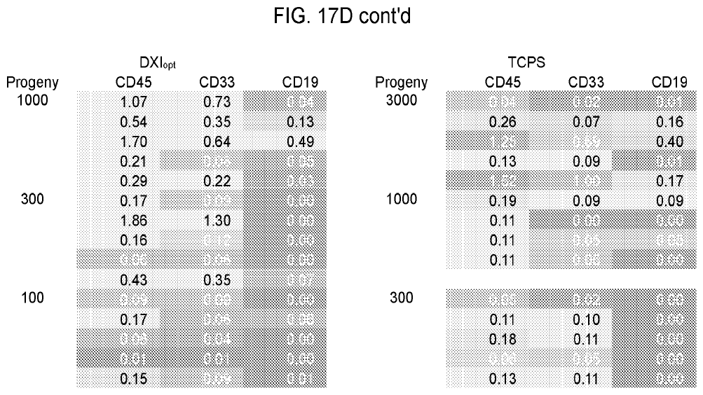

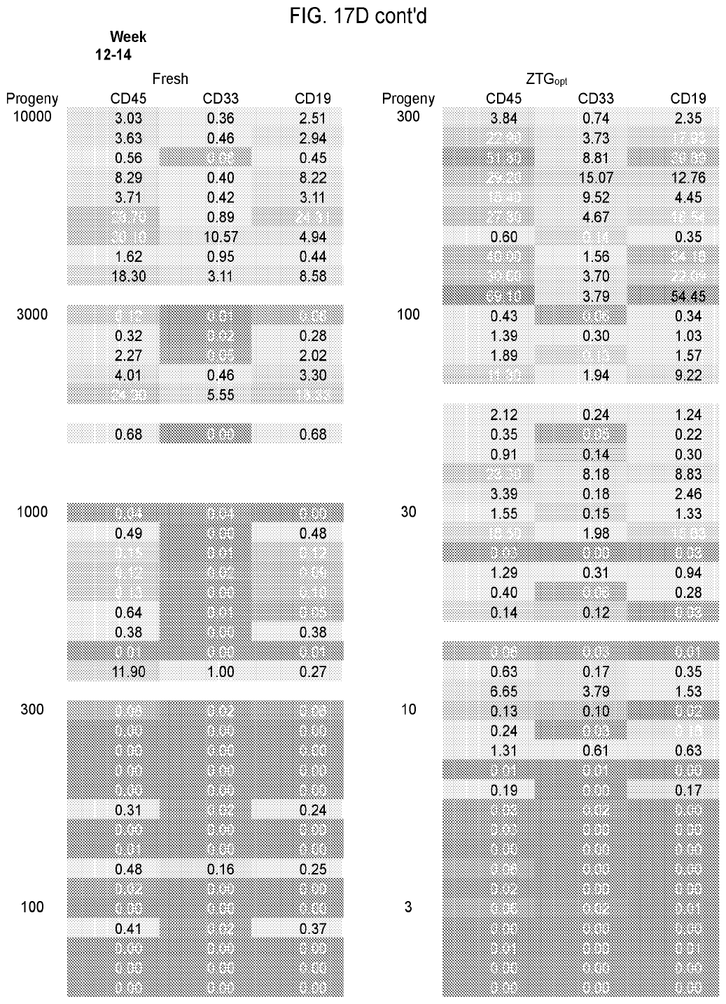

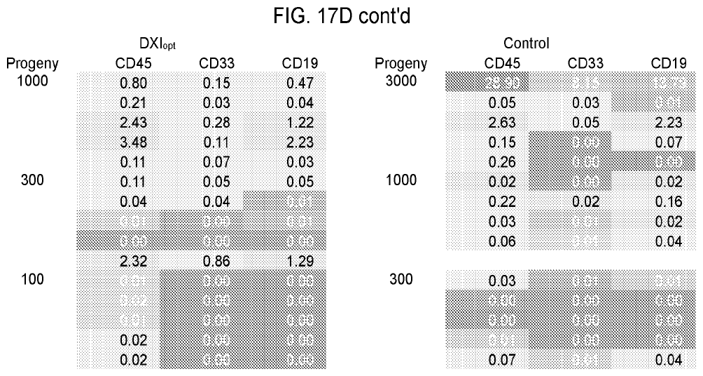

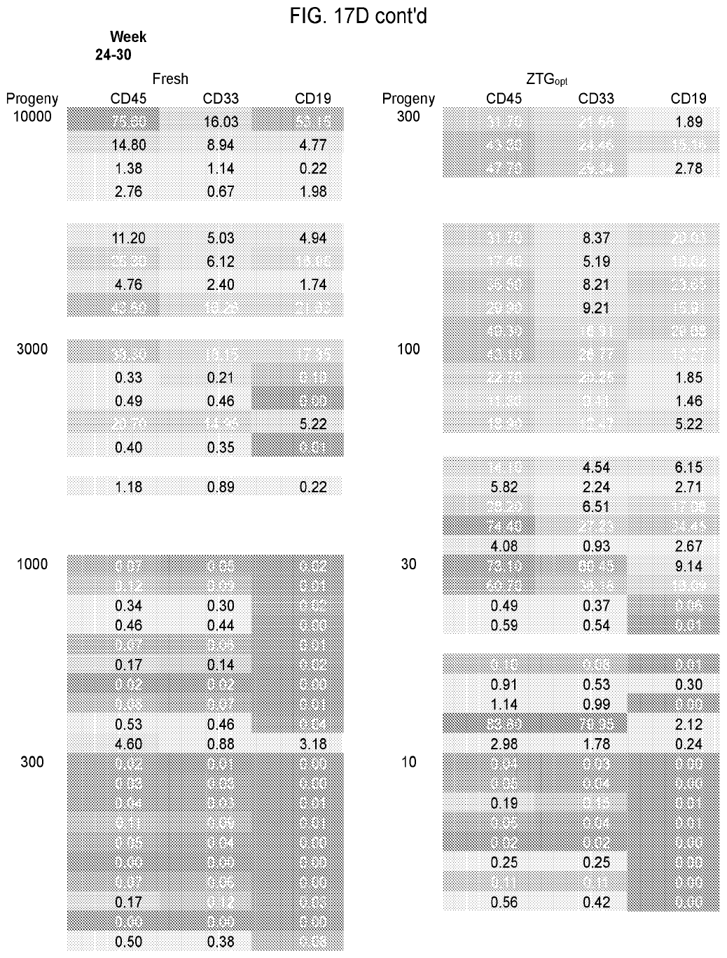

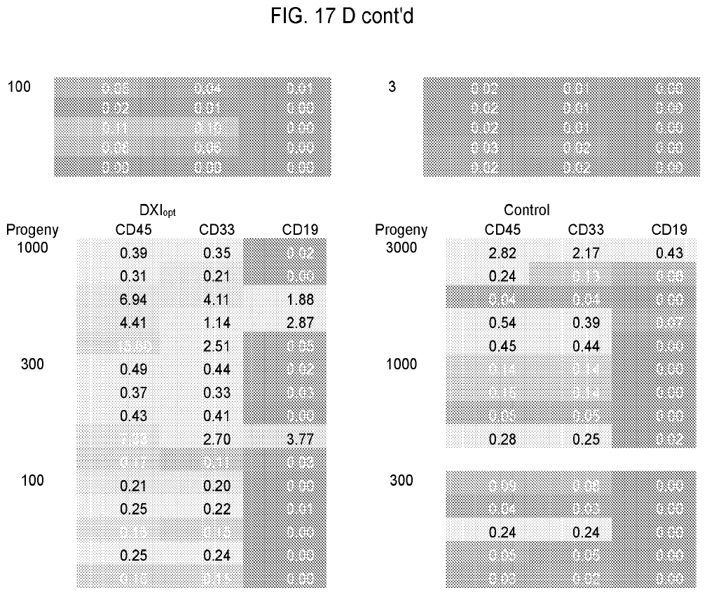

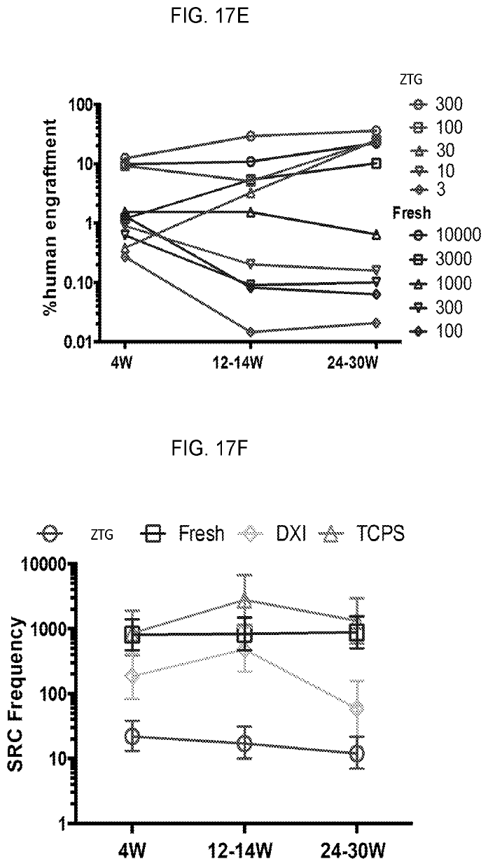

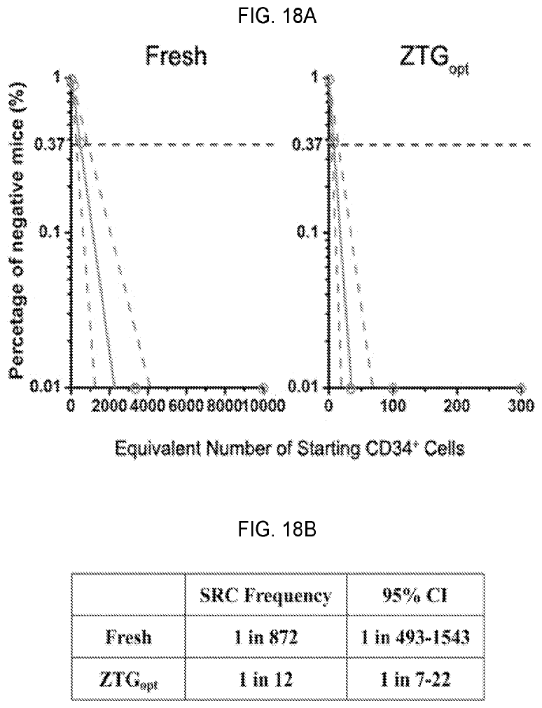

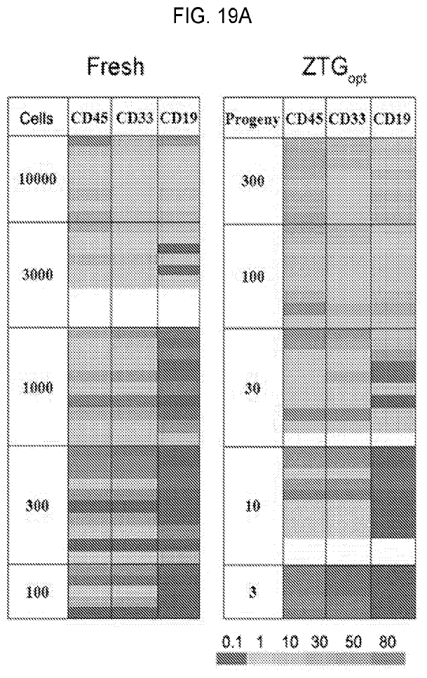

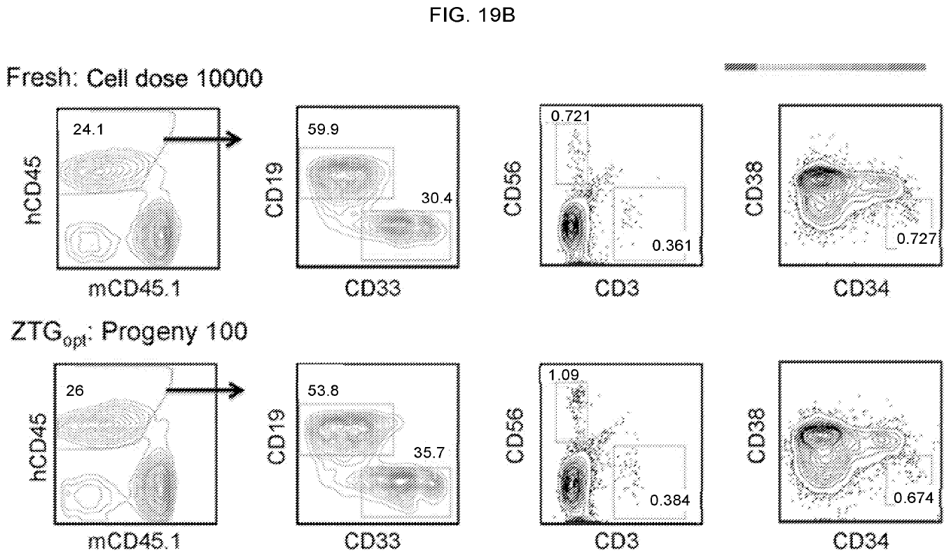

Having demonstrated that CB-derived CD34+ cells cultured with the ZTGₒₚₜ method maintained their in vivo repopulating ability, limiting dilution analysis (LDA) was next conducted to determine the frequencies (adjusted to the numbers of CD34+ cells at day 0) and absolute numbers of long-term repopulating HSC (LT-HSC), comparing ZTGₒₚₜ cultured cells with non-cultured cells (FIG. 17A-17F) (Ito et al., Blood, 100:3175 (2002)). When analyzed at 24 and 30 weeks post-transplantation, the frequencies of LT-HSGs in ZTGₒₚₜ expanded cells were measured at 1 per 12 CD34⁺ starting cells (95% confidence interval of 6.8 to 21.5), whereas in non-cultured CD34⁺ CB cells, they were measured at 1 per 880 CD34⁺ starting cells (95% confidence interval of 495 to 1542) (FIGS. 18A and 18B). This represented a 73-fold increase in frequencies of day 0 equivalent LT-HSC in ZTGₒₚₜ cultured cells when compared with non-cultured cells (FIG. 18B), and a 5-fold increase over DXIₒₚₜ expanded cells ( 1/59), and a 110-fold increase over the control-expanded cells ( 1/1320) (FIG. 17C). At all times evaluated (short (4 weeks), mid (12-14 weeks), and long (24-30 weeks) term post-transplant) ZTGₒₚₜ expanded cells demonstrated increased levels of human engraftment than non-cultured CD34+ cells as well as those expanded in DXIₒₚₜ and control cultures (FIGS. 17B-17D). Notably, a ZTGₒₚₜ-expanded population generated from 100 fresh CD34+ cells had a similar level of sustained engraftment in NSG mice (24.7%) as 10,000 uncultured CD34+ cells (22.4%) at 24-30 weeks post-transplantation (FIGS. 19A and 19B). In addition, both lymphoid and myeloid engraftment were detected in mice that received the non-cultured and ZTGₒₚₜ-cultured cells (FIG. 19A).

It was next examined whether ZTGₒₚₜ-expanded cells retained the multi-lineage potential of the starting non-cultured HSPC. To exclude the impact of different graft levels on lineage contribution, two groups that had similar human engraftment were compared: the group receiving ZTGₒₚₜ cells derived from the expansion of 100 fresh HSPC, and the group receiving 10,000 fresh HSPC. The level of human engraftment was similar in both groups, indicating that ZTG expanded populations retained full HSPC multi-lineage repopulation capabilities (FIG. 19B). Importantly, similar levels of CD34⁺CD38⁻ (0.73% vs 0.67%) cells were observed, signifying the continued presence of LT-HSCs in the BM of NSG recipients (FIGS. 17B-17D, 19B).

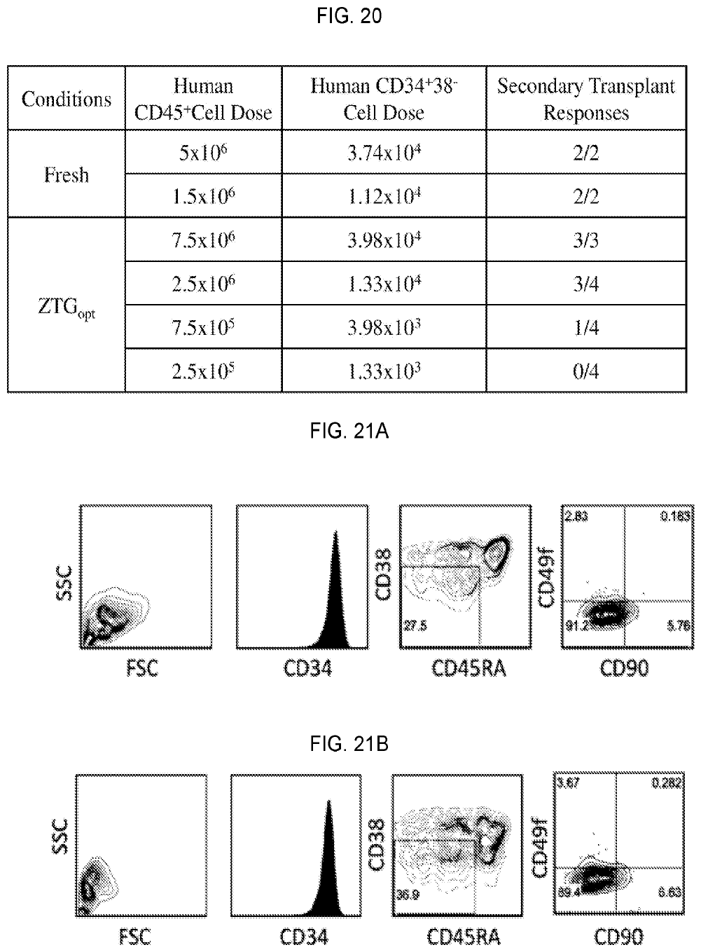

In addition to LDA, secondary transplants were conducted to further determine the presence of LT-HSC engraftment. Primary recipients transplanted with ZTGₒₚₜ cells were sacrificed at 24 weeks post-transplantation and half of their marrow was transplanted into secondary mice. Human engraftment (defined as >0.1% human CD45⁺ cells) was observed in the marrow of 4 out of 5 secondary recipients (FIG. 20), supporting the sustained presence of an LT-HSC population and showing that ZTG-expanded HSPC remained competent in secondary recipients.

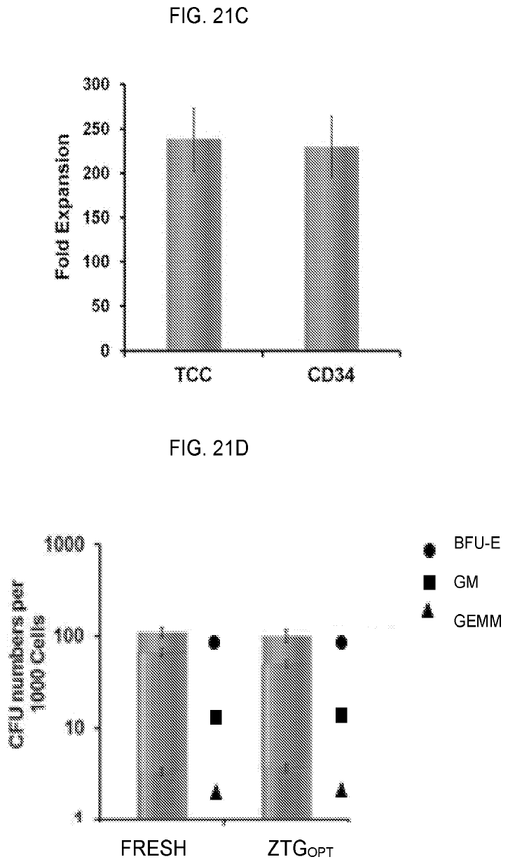

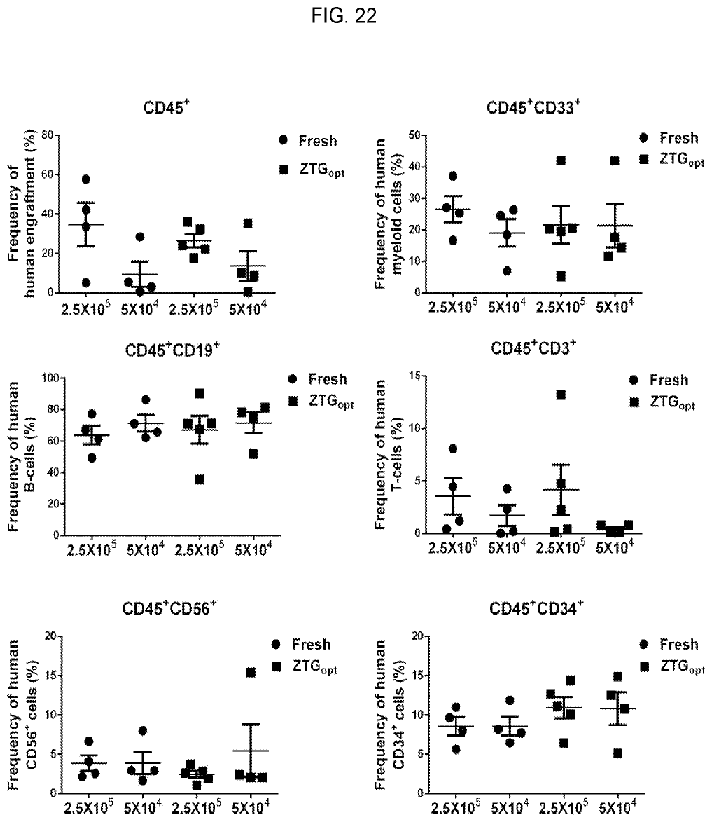

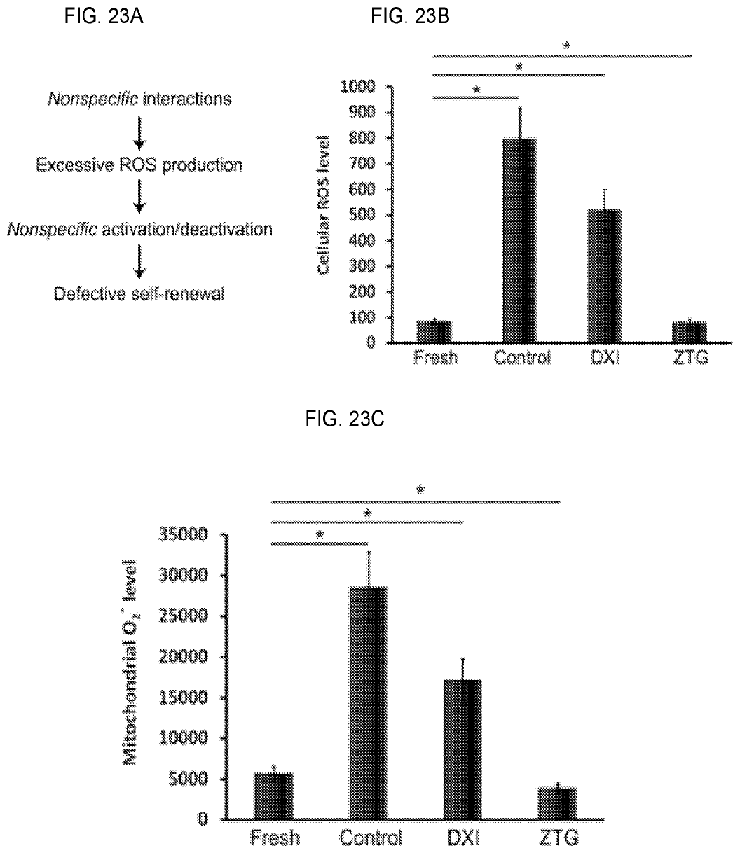

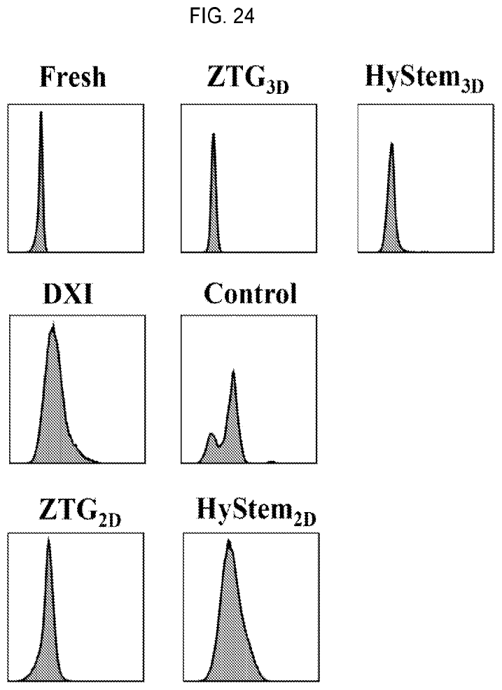

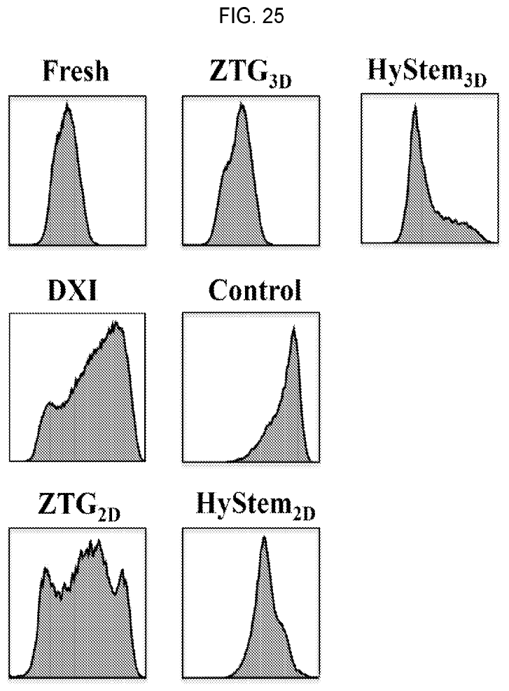

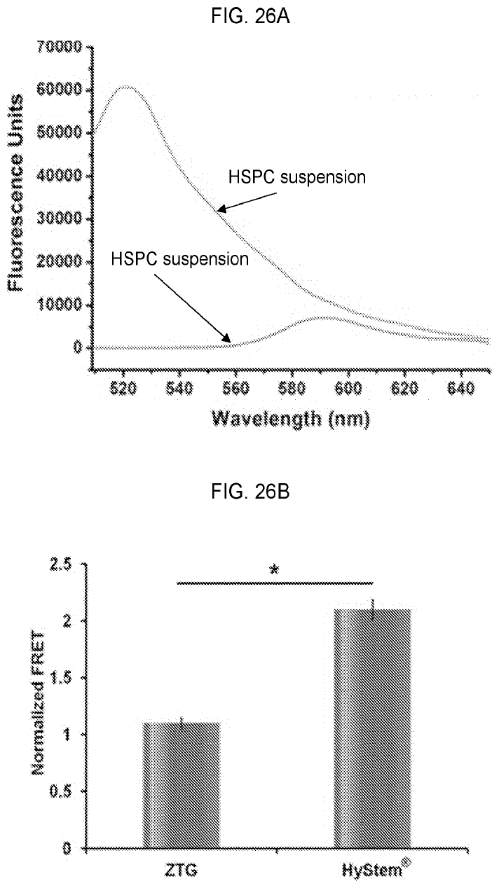

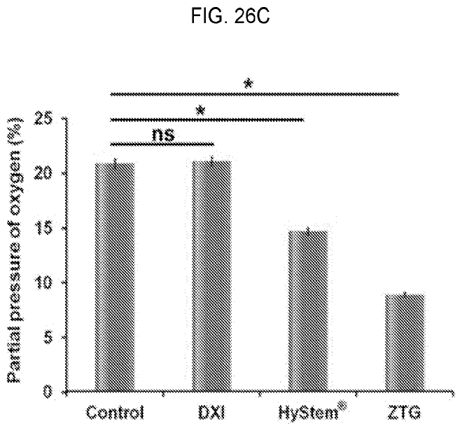

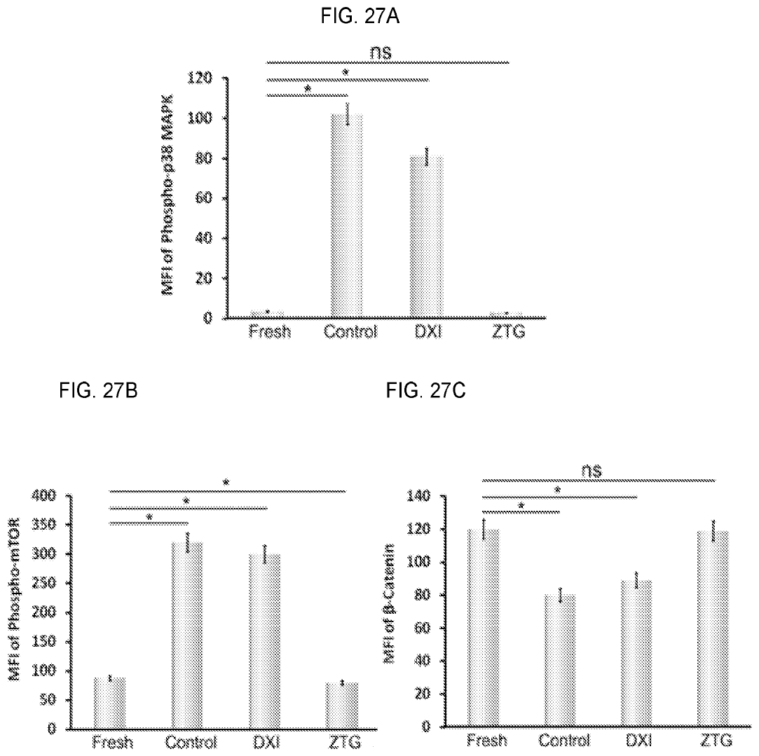

After demonstrating the ability of the ZTGₒₚₜ method to support the ex vivo expansion of CB-derived HSPC with primitive phenotypes and in vivo functionality, this same strategy was applied to expand BM-derived HSPC ex vivo. Similar to ZTGₒₚₜ cultures initiated with CB HSPC, ZTGₒₚₜ expansion of BM-derived HSPC inhibited differentiation while promoting expansion and maintenance of cells expressing primitive markers (FIGS. 21A and 21B). ZTGₒₚₜ cultures initiated with BM-derived HSPC resulted in a 238-fold TCC increase which was again nearly matched by the 230-fold increase of CD34+ cells (FIG. 21C). Both in vitro and in vivo functional assays demonstrated strong ZTGₒₚₜ repopulating capacity and no significant difference was found between fresh and ZTGₒₚₜ groups (FIGS. 21D and 22). All of these results corroborate the CB-derived cell findings. Possible mechanisms by which the non-fouling ZTG culture environment influences HSC expansion activity, and promotes self-renewal over differentiation decisions was next explored. Biomaterials are known to induce production of ROS, which leads to a number of possible pathophysiological outcomes including cytotoxicity and foreign body reactions such as fibrosis, atherogenesis, and granulomas (Nel, et al., Science, 311:622 (2006); Kaplan, et al., J Biomed Mater Res, 26:1039 (1992)). Reactive oxygen species can nonspecifically react with a number of redox-sensitive molecules, resulting in oxidative modifications including cysteine oxidation, cysteine nitrosylation, cysteine glutathionylation, methionine oxidation, protein carbonylation, and protein hydroxylation (Bigarella, et al., Development, 141:4206 (2014)). These oxidative modifications can directly or indirectly affect the function and activation of transcription factors (e.g. EOXO, p53, PRDM16, NRF2, HIF et al.), as well as kinases (e.g. mTOR, p38 MAPK, AKT et al.) and phosphatases (e.g. PTEN) (Bigarella et al., 2014, supra; Ray et al., Cell Signal, 24:981 (2012)). Other regulators, such as the signal transducer β-Catenin, the cytokine signaling inhibitor LNK, the modulator KEAP1, the E3 ubiquitin ligase MDM2, and the cell cycle inhibitors p16INK4A and p19ARF, can also be affected by ROS levels. It has been reported that increased ROS levels can inhibit HSC self-renewal pathways such as Wnt/β-Catenin (Shin et al., Cancer Lett, 212:225 (2004); Reya et al., Nature, 423:409 (2003)) while activating pathways that can result in defective self-renewal such as p38 MAPK (Ito et al., Nat Med, 12:446 (2006)), mTOR (Yoshida et al., J Biol Chem, 286:32651 (2011)), etc. (Ito et al., Nature, 431:997 (2004)). These ROS-induced pathway activation and deactivation processes appear to be nonspecific. In nature, primitive HSPCs reside in a low-oxygenic niche that limits ROS production and provides the cells with long-term protection (Jang and Sharkis, Blood, 110:3056 (2007)). Here, whether ZTG culture can also limit ROS production and protect the encapsulated HSPCs was addressed. Since the early work by Langer and others (Liu et al., Biomaterials, 32:1796 (2011); Yu et al., J Biomed Mater Res A, 103:2987 (2015)), researchers have noticed a significant difference in ROS production between hydrophilic and hydrophobic culture systems. Hydrophobic culture experiences excessive ROS production while hydrophilic culture is able to significantly reduce this. This was attributed to changes in protein conformation as they nonspecifically interact with a hydrophobic surface, which disturbs cell membranes and induces cellular ROS production (Yu et al., 2015, supra). Without being bound by theory, culture in hydrophobic environments may provide cells with nonspecific interactions and induce excessive ROS production; as a result, these excessive ROS may nonspecifically activate HSPC differentiation pathways while inhibiting HSC self-renewal pathways (FIG. 23A). In contrast, the lack of nonspecific interactions in ZTG cultures may inhibit ROS-induced nonspecific pathway activation/deactivation, enabling differentiation-free HSC expansion to be achieved in ZTG cultures. In this work, the cellular response was measured after one day of culture in each system, which is sufficient for HSPCs to respond to environment changes while cell differentiation is minimized. As presented in FIGS. 23B, 23C, cellular ROS and mitochondrial superoxide levels slightly decreased in the ZTG group, while significant increase was observed in both DXI and control groups (FIGS. 24, 25). In addition, increased ROS production in HYSTEM® culture (FIGS. 24, 25), due to stronger nonspecific interactions between HSPCs and the hydrogel matrix (FIGS. 26A, 26B), was detected. Moreover, the ZTG slowed down oxygen transport, since water, the oxygen carrier water, is strongly bound to the hydrogel matrix and impedes oxygen mass transport. This further limits the source of ROS production in ZTG culture (FIG. 26C). The activation or deactivation of ROS-related signaling pathways in each culture system was examined. Inhibition of the p38 MAPK (Wang et al., Stem Cells Dev, 20:1143 (2011)) and mTOR (Huang et al., Nat Med, 18:1778 (2012); Luo et al., Transplantation, 97(1):20 (2014)) pathways, along with activation of the Wnt/β-Catenin (Fleming et al., Cell Stem Cell, 2:274 (2008)) pathway, have been demonstrated to significantly enhance HSC self-renewal. In the analysis of these culture platforms, increased ROS levels clearly correlate with increased p38 activation (FIG. 27A) in the DXI and control cultures, likely leading to defective self-renewal (Ito et al., 2006, supra). In addition, DXI and control cultures exhibited β-Catenin and mTOR signaling behavior opposite to that expected for healthy self-renewal, with β-Catenin exhibiting reduced expression and mTOR showing significant activation in these cultures (FIGS. 27B, 27C) (Huang et al., 2012, supra). On the other hand, the inhibited production of excessive ROS in the ZTG culture was found to correlate with the desired pathway responses, enhancing self-renewal and avoiding defective self-renewal behavior.

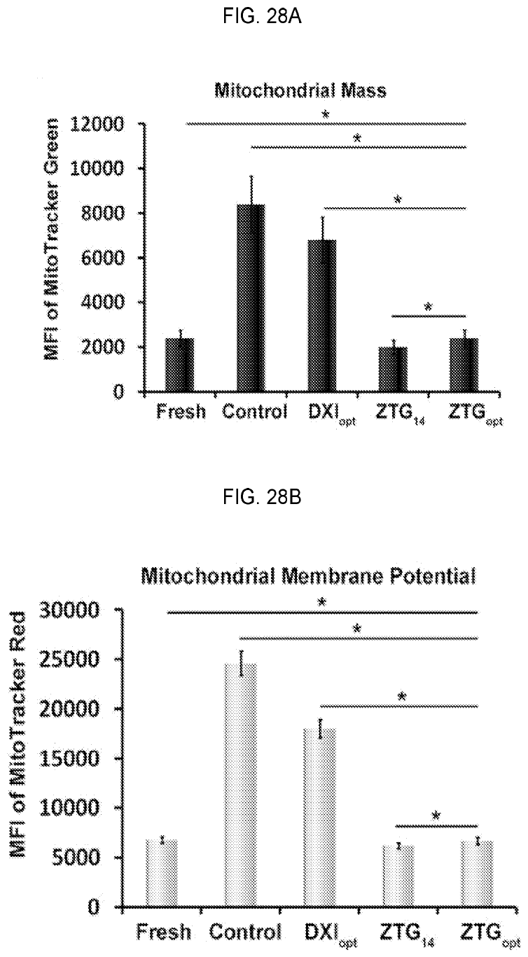

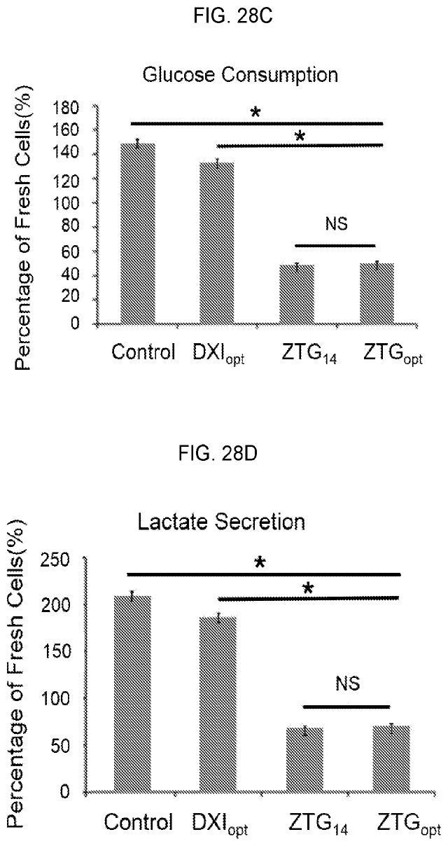

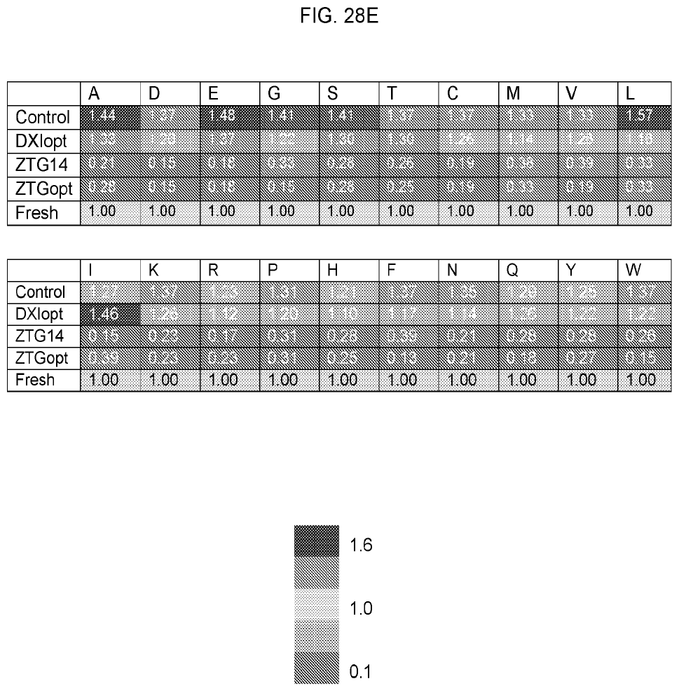

Mitochondrial mass and membrane potential was further analyzed, as well as metabolic activity markers, in the 14-day ZTG (ZTG₁₄), 24-day ZTG (ZTGₒₚₜ), and other expanded populations. As presented in FIGS. 28A and 28B, mitochondrial mass and mitochondrial membrane potential marginally decreased in ZTG₁₄ and ZTGₒₚₜ cells—these results sharply contrast the significant increases in both mass and membrane potential observed in the DXIₒₚₜ and control groups, which was again correlated with excessive ROS production in these platforms. Moreover, since metabolic activity is another key regulator of HSC self-renewal (McGraw et al., Nat Chem Biol, 6:176 (2010); Ito et al., Nat Rev Mol Cell Biol, 15:243 (2014)), glucose consumption, lactate secretion, and amino acid metabolism was measured in all expansion cultures. After 14 days, cells in the control and DXIₒₚₜ systems exhibited significantly higher glucose consumption and lactate secretion than ZTG cells, which correlated to the increased energy required for differentiation and the specialized functions of differentiated progeny (FIGS. 28C and 28D). In comparison, no significant changes in these metabolic activities were found in ZTG₁₄ (cells cultured for 14 days) and ZTGₒₚₜ cells. HSC from each system were further tested for levels of the twenty canonical amino acids necessary for polypeptide biosynthesis, using a triple quadrupole (QqQ) LC/MS. The signal from each amino acid was first normalized to the total DNA content and then to the corresponding signal from fresh HSPC. Moderately upregulated biosynthesis in DXIₒₚₜ and control cultures, but sharply downregulated biosynthesis in ZTG-expanded cells (FIG. 28E) was found. Again, no significant difference was found between ZTG₁₄ and ZTGₒₚₜ cells, suggesting that HSC are able to slow their metabolism in the non-fouling environment of a ZTG. Without being bound by theory, it is thought that the ability of the stem and progenitor cells to reduce metabolism allows them to remain in a naïve state for an extended period.

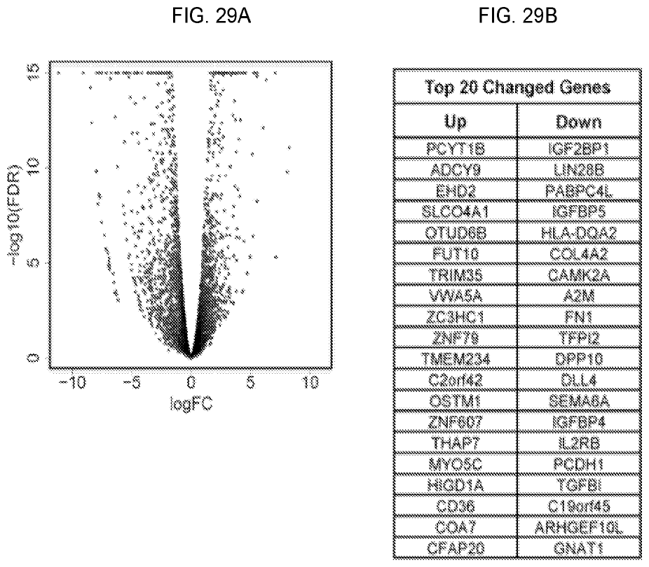

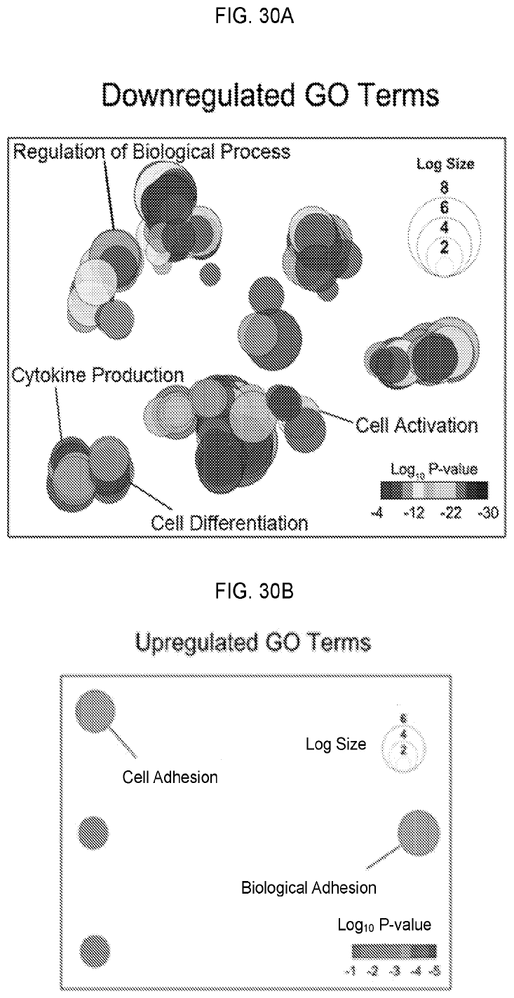

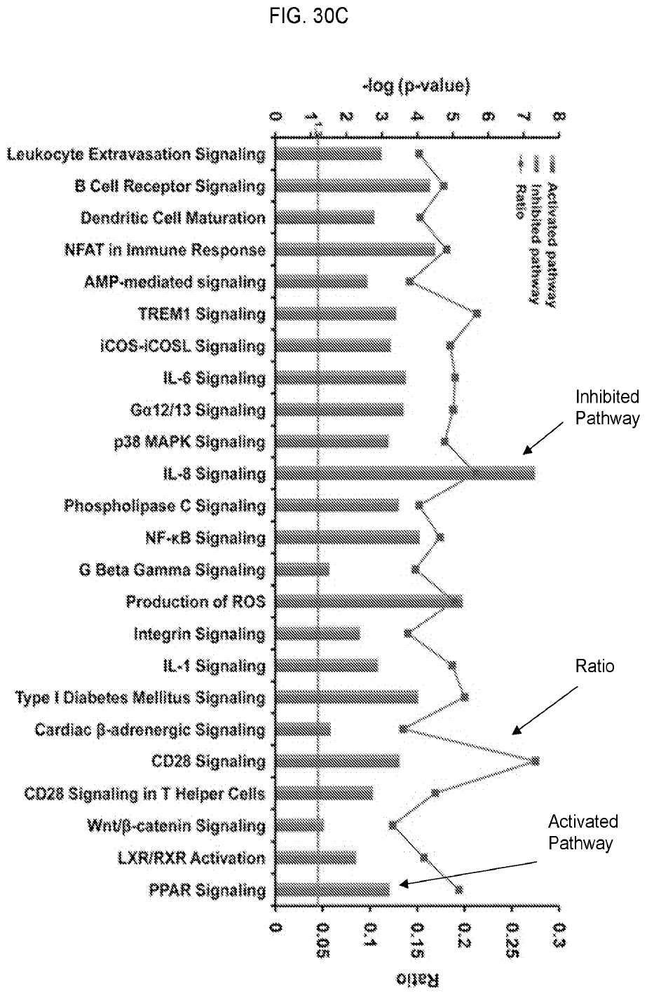

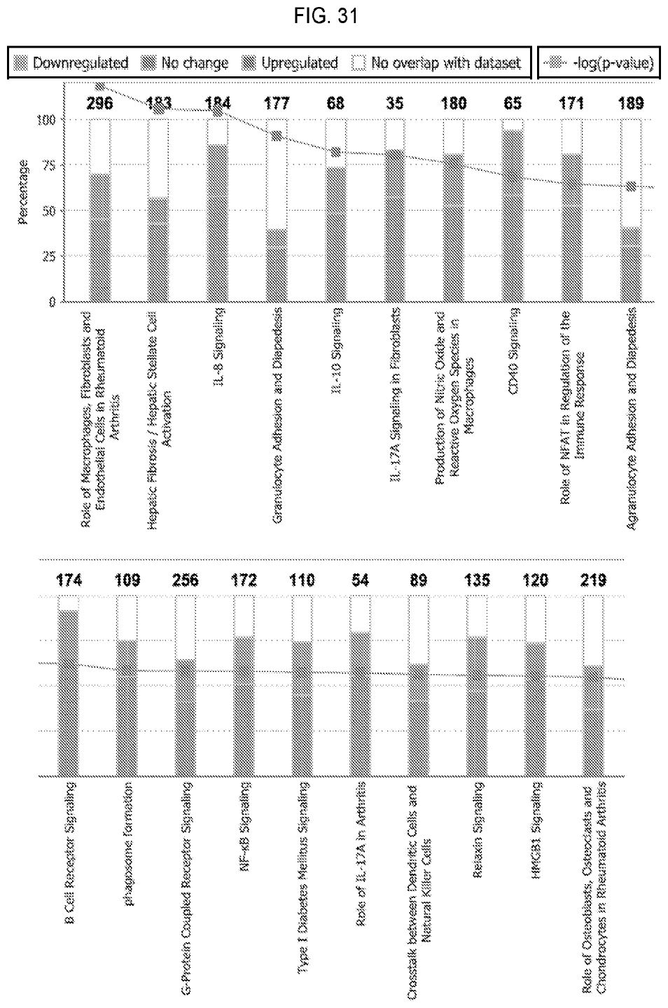

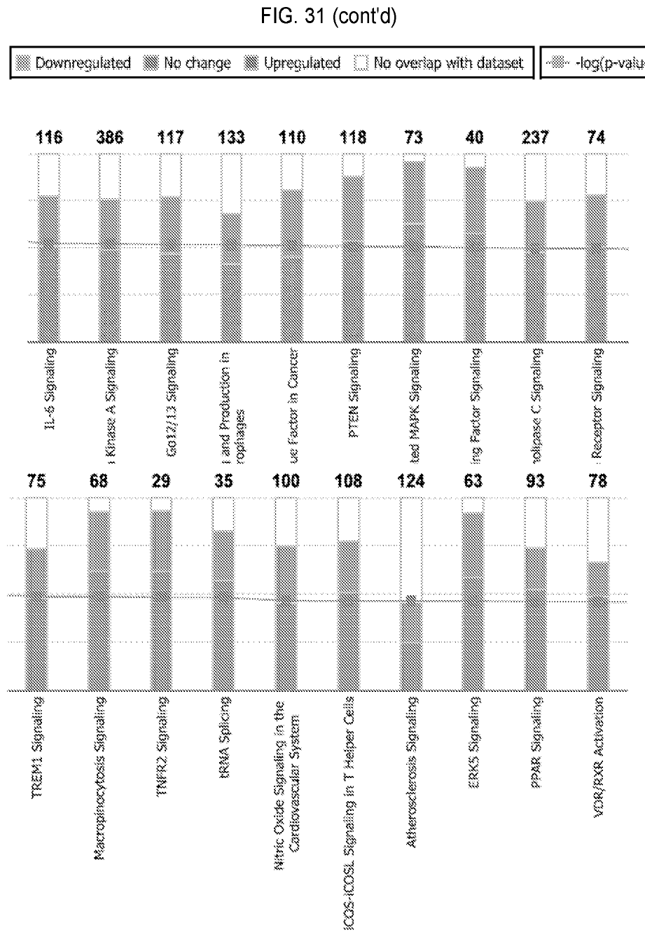

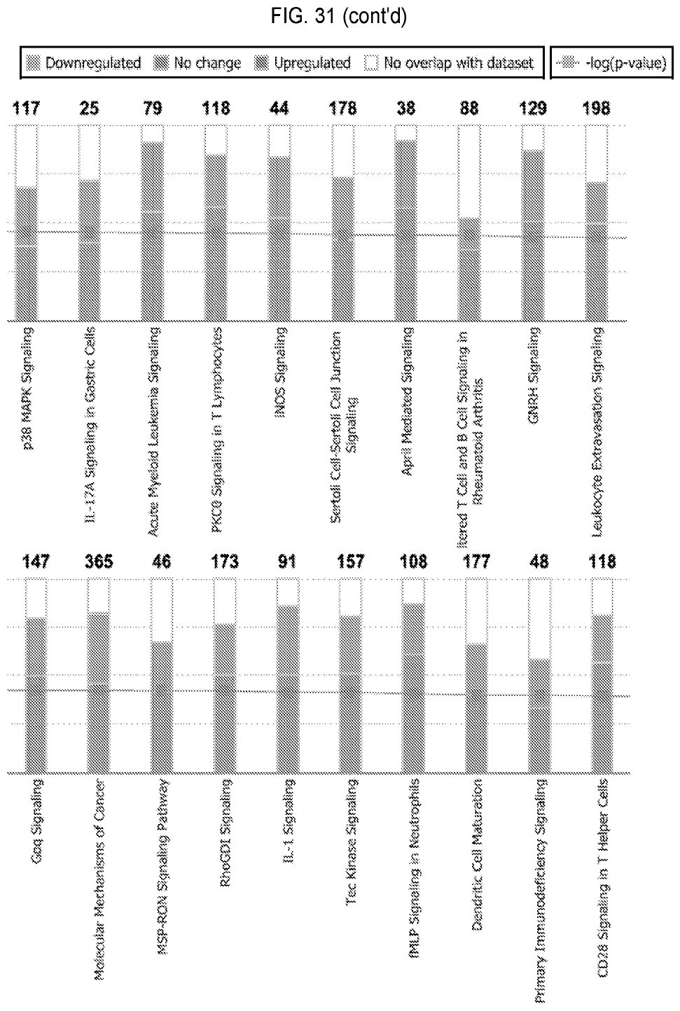

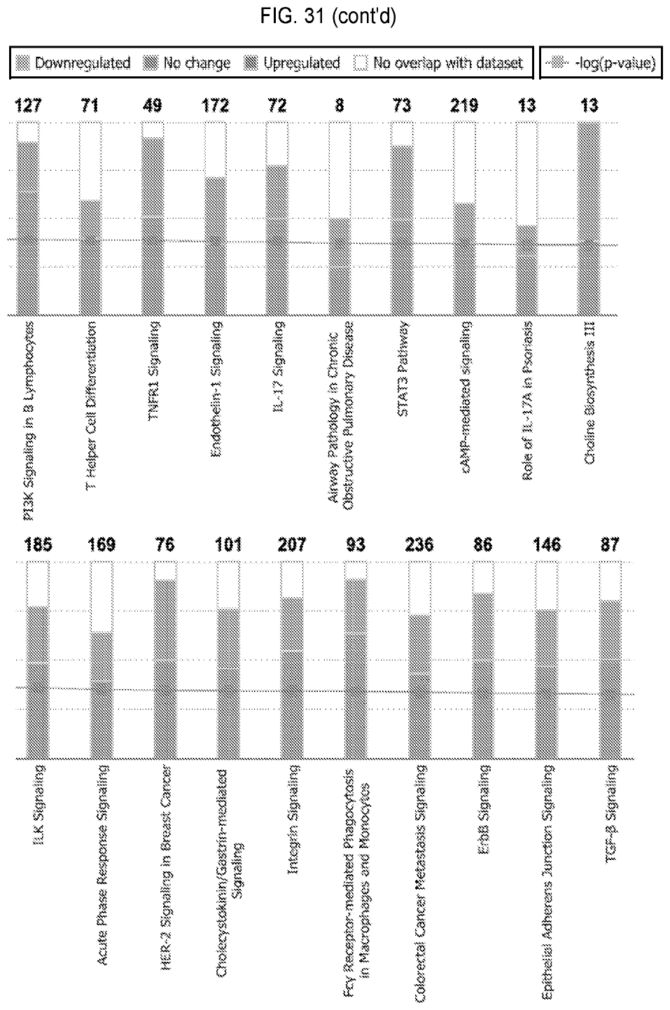

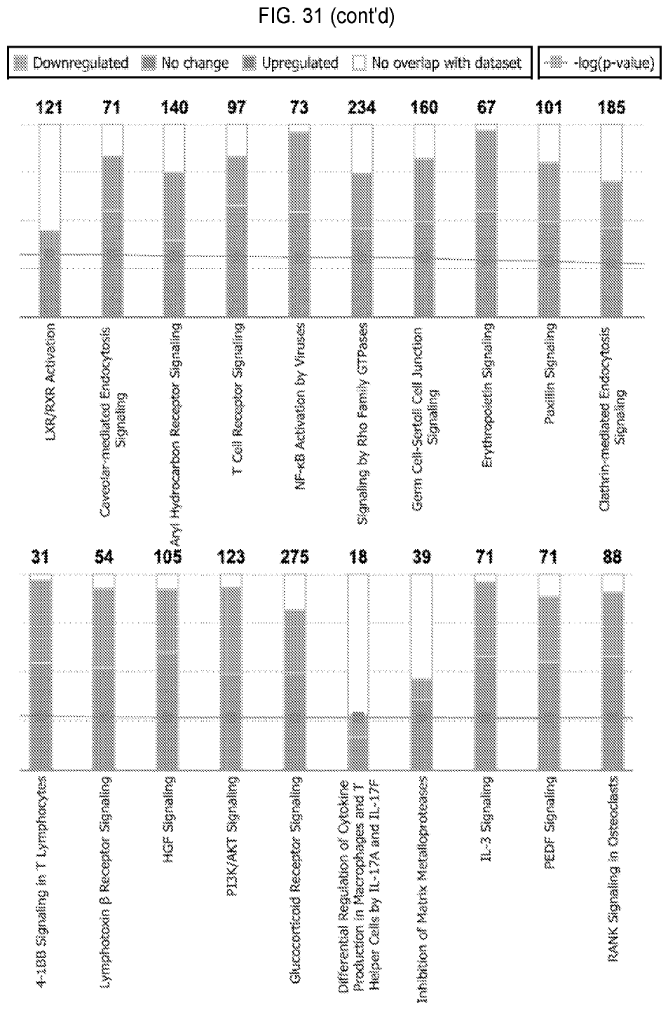

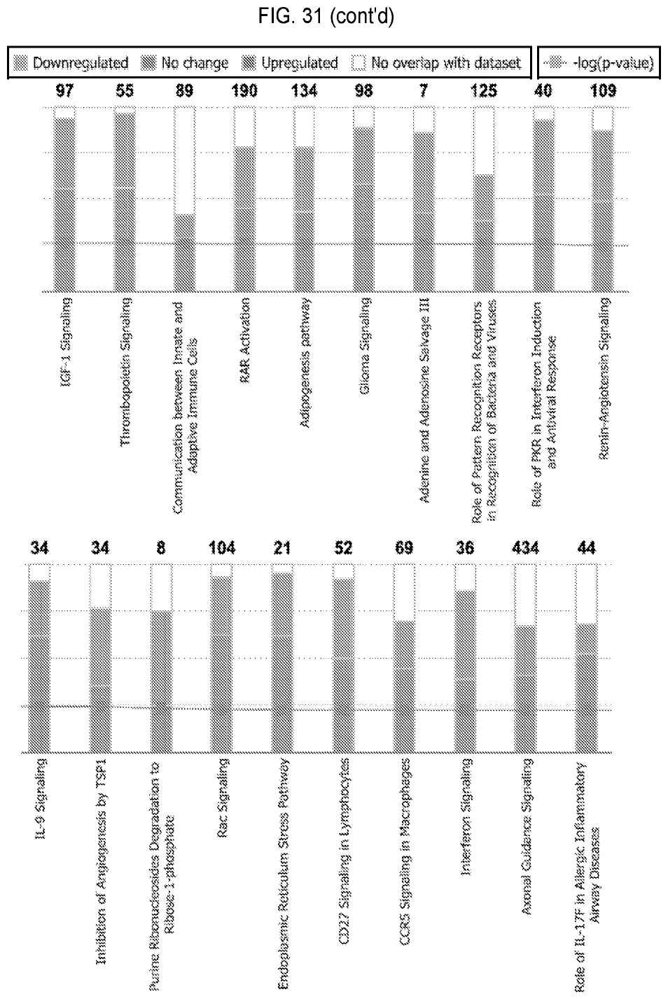

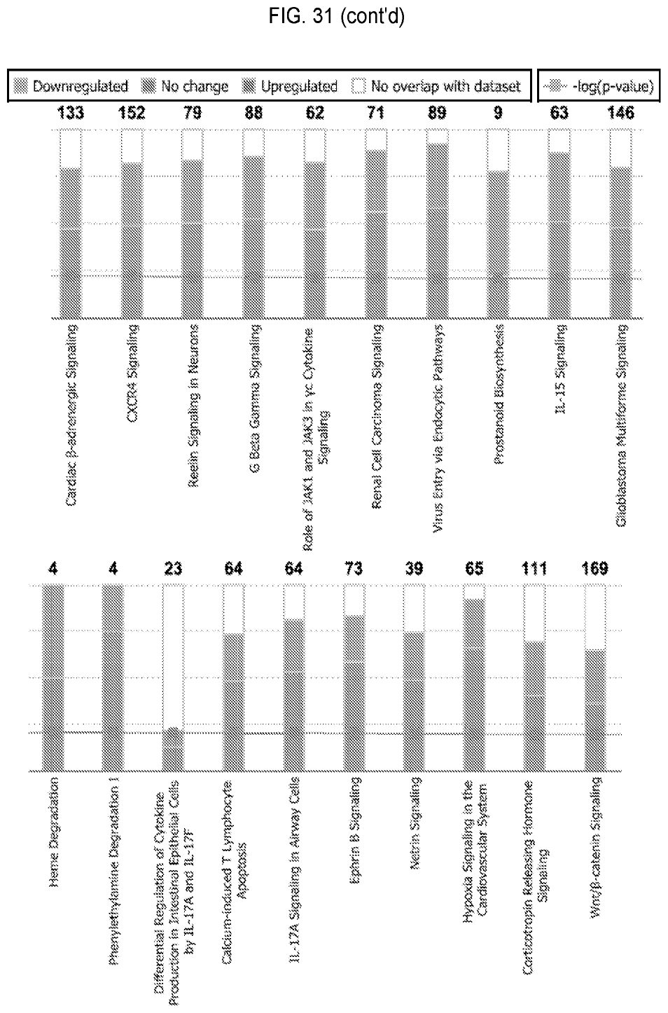

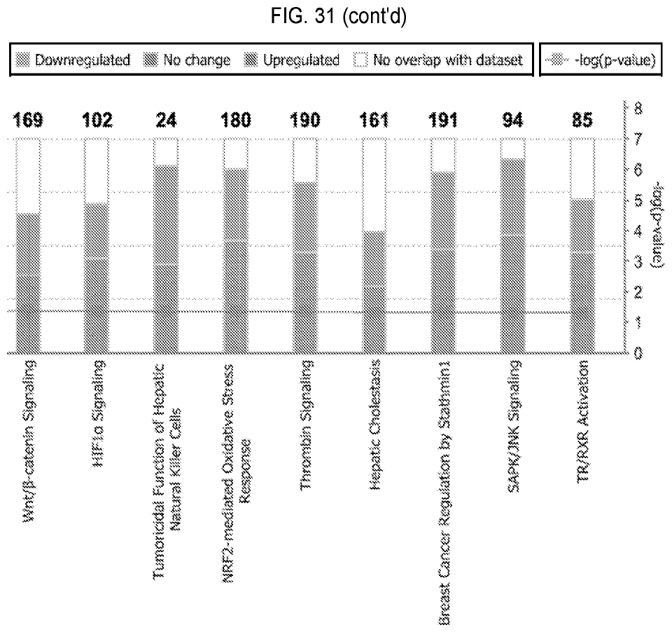

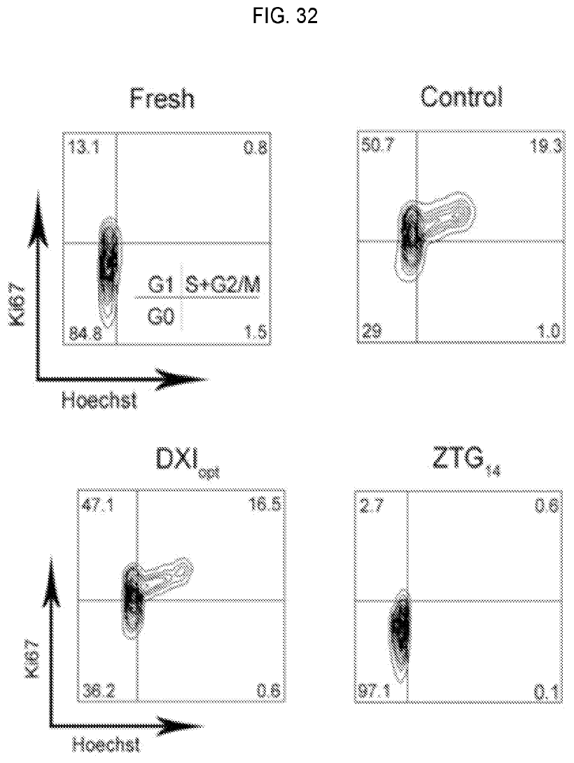

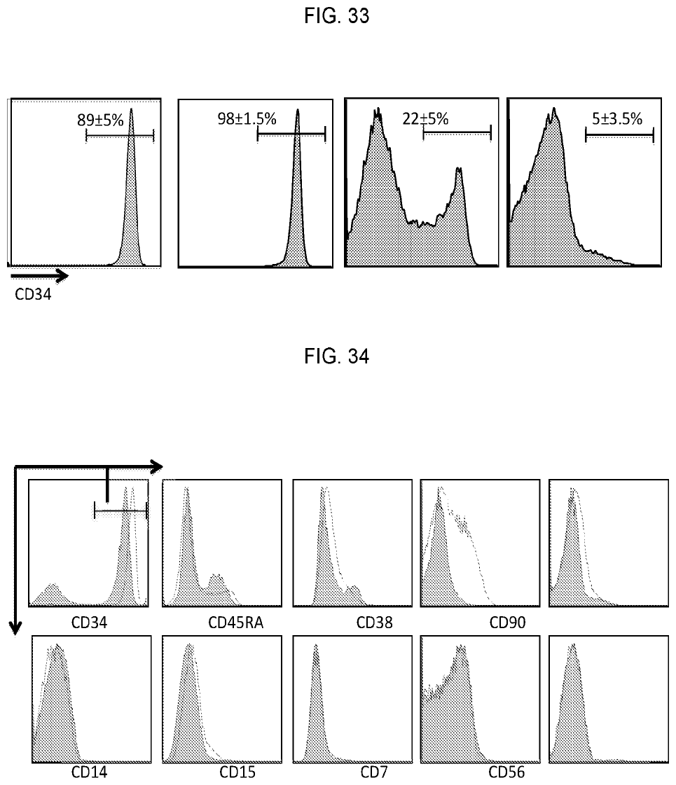

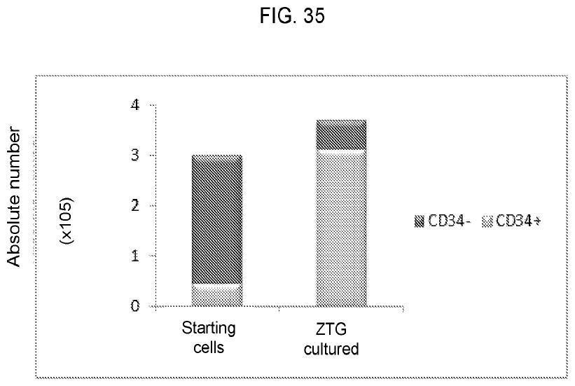

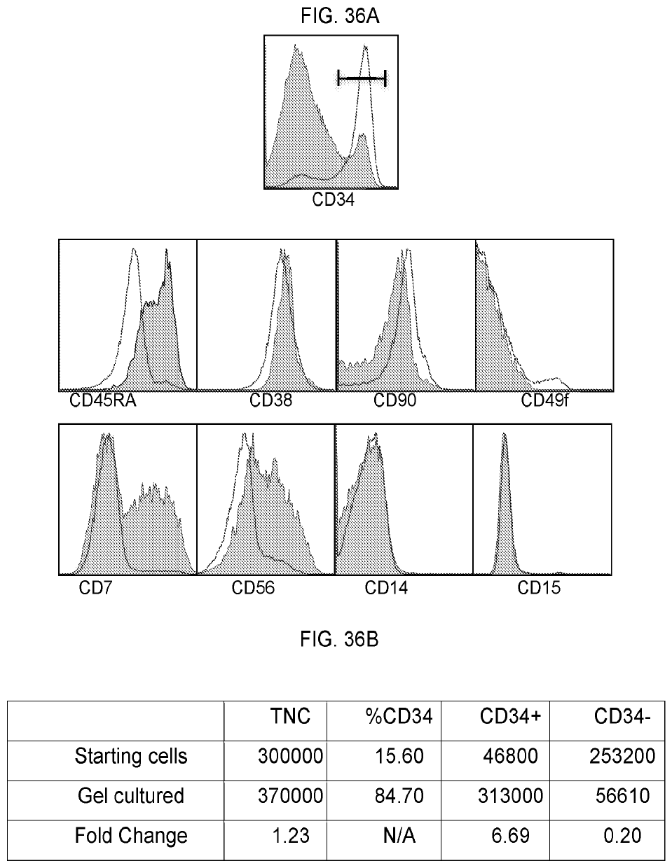

Finally, for more advanced insight on the activity of CD34+ hematopoietic cells in a purely zwitterionic background, expression profiling was conducted employing mRNA deep-sequencing (RNA-seq). As presented in FIGS. 29A and 29B, while 778 genes were found to be significantly up-regulated and 926 genes were found to be significantly down-regulated, expression of more than 10,000 genes did not show significant change in ZTG-expanded cells compared to fresh HSPCs. Gene ontology (GO) enrichment analysis of the biological process terms (GO:BP) for the up- and down-regulated gene sets was done (FIGS. 30A, 30B). After compressing GO terms, 875 general GO:BP terms were found to be statistically enriched in the down-regulated gene set, including those for cell differentiation, cell activation and cytokine production, again indicating slowed metabolic processes. Strikingly, only 4 terms, all associated with cell adhesion, were found statistically enriched in the up-regulated gene set, indicating the encapsulated cells were trying to adapt to their new niche. Similar to the GO enrichment analysis, a complete canonical pathway analysis predicted activation of three pathways in ZTG expanded cells (FIG. 30C), including self-renewal-related pathways such as Wnt/β-Catenin, LXR/RXR and PPAR signaling (Ito et al., 2014, supra). Furthermore, inhibition was predicted for twenty-one pathways in this platform, again including several tied to self-renewal—p38 MAPK signaling (Ito et al. 2006, supra), ROS production (Ray et al., Cell Signal, 24:981 (2012)), Gα12/13 signaling (Nishida et al., J Biol Chem, 280:18434 (2005)) and others. Inhibition of AMP-mediated signaling and IL-6 signaling are correlated with the reduced metabolism in growing cells from ZTG culture (Long et al., J Clin Invest, 116:1776 (2006); Carey et al., Diabetes, 55:2688 (2006)). A complete canonical pathways analysis can be found in FIG. 31.

The findings establish that HSPC culture in ZTG promotes significant and clinically meaningful expansion of CB and BM-derived HSC with long-term repopulating ability while substantially reducing and perhaps even blocking their differentiation ex vivo. This methodology therefore has clinical implications beyond increasing the absolute number of CD34+ cells for use in CBT, and will also be useful in gene therapy settings in which a genetically modified/corrected HSC can be expanded prior to infusion. This work complements previous efforts to improve HSPC expansion in vitro, including through fed-batch culture (Csaszar et al., Cell Stem Cell, 10:218 (2012)), aryl hydrocarbon antagonists (Boitano et al., Science, 329:1345 (2010)), the UM family of molecules (Fares et al., Science, 345:1509 (2014)), Notch ligand signaling (Delaney et al., Nat Med, 16:232 (2010)), copper chelators (De Lima et al., Bone Marrow Transplant, 41:771 (2008)) or the expression of HOX genes (Antonchuk, Cell, 109:39 (2002))).

Materials and Methods.

Synthesis of bromine end-functional star-shaped zwitterionic polymers. Br-terminated star-shaped pCBAA polymer was produced by atom-transfer radical-polymerization (ATRP) as reported in Lutz, et al., Macromolecules, 39:6376 (2006). In brief, CBAA-1 (46 mmol), 2,2′-bipyridine (bpy, 1.6 mmol), CuBr (0.48 mmol), CuBr₂ (0.32 mmol) and tetrafunctional initiator, pentaerythritoltetrakis (2-bromoisobutyrate) (0.37 mmol) were dissolved in CH₃CN/H₂O (3:7) solution and placed in a 10-mL reaction tube, and the mixture was subjected to three freeze-pump-thaw cycles. The reaction was allowed to continue at room temperature under stirring for 8 hr. The polymer product was recovered after treatment with alumina, and finally purified by dialysis. Final polymer was obtained via lyophilization. Star-shaped polymers with target molecular weights of 28,000 were synthesized.

Synthesis of azide end-functional star-shaped zwitterionic polymers. The bromine end-functional pCBAA (4 g, 0.2 mmol) and sodium azide (65 mg, 1 mmol) were dissolved in 5 mL dimethylformamide/H₂O (9:1) solution and stirred at 50° C. for 24 hr. The final mixture was diluted with deionized water and subsequently purified by dialysis against pure water. Polymer was obtained via lyophilization. The efficiency of the reaction was calculated by ¹H-NMR.

Synthesis of NH₂-functionalized star-shaped pCBAA polymers. The azide group on this star-shaped pCBAA was converted into NH₂ by using a ‘click’ reaction. N₃-pCBAA (2 g, 0.1 mmol) and 1-amino-3-butyne (65.7 mg, 0.95 mmol) were first dissolved in 10 mL degassed mixing solvent of methanol and water (1:2). Then, CuBr (67.4 mg, 0.47 mmol) and N,N,N′,N″,N″-pentamethyldiethylenetriamine (PMDETA; 164.6 mg, 0.95 mmol) were separately added into the above solution. The mixture was stirred under a nitrogen atmosphere at room temperature and degassed by three freeze-pump-thaw cycles. Then, the tube was kept at room temperature for 48 hr. Small molecules were removed via dialysis and the polymer was obtained via lyophilization. The efficiency of the reaction was calculated by ¹H-NMR.

Synthesis of click-functionalized star-shaped pCBAA polymers. As reported in DeForest & Anseth, Nat Chem, 3:925 (2011), DIFO₃ was functionalized to the end of star-shaped pCBAA polymer. In brief, appropriate amounts of N-(3-dimethylaminopropyl)-N′-ethylcarbodiimide hydrochloride (EDC) (Sigma) and N-hydroxysuccinimide (NHS) (Sigma) were added to DIFO₃ solution. After incubation at 25° C. for 1 hr to activate the carboxylate group, (1 g, 0.05 mmol) NH₂-terminated polymer was added to the activated system. The one-carbon spacer between the positive charged group and negative charged group avoids activation of the carboxyl acid group on CBAA-1. The concentration of DIFO₃ in the reaction solution was set at 100 μM. The molar ratio of EDC, NHS and DIFO₃ was fixed at 1:1:1. The reaction was allowed to proceed at 25° C. for 24 hr before purification. The efficiency of the reaction was calculated by ¹H-NMR.

Preparation of self-assembled monolayers. Glass chips were first coated with an adhesion-promoting chromium layer (thickness 2 nm) and a surface-plasmon-active gold layer (48 nm) by electron beam evaporation under vacuum. Before self-assembled monolayer (SAM) preparation, the gold-coated glass substrate was rinsed with ethanol and water in sequence, dried with filtered air, then further cleaned in a UV ozone cleaner (Jelight, model 42) for 20 min. The cleaned chip was immediately soaked in a 0.1 mM ethanol solution of ATRP initiator for 24 h to form a SAM on the gold surface (Zhang, et al., J Phys Chem B, 110:10799 (2006)). The chip was subsequently rinsed with THF, then ethanol, and dried with a stream of filtered air just prior to surface-initiated polymerization.

Surface-initiated atom transfer radical polymerization. Surface-initiated ATRP was carried out on SAM-coated gold substrates following a method similar to one previously reported (Zhang, et al., J Phys Chem B, 110:10799 (2006)). Briefly, CuBr, CuBr₂, BPY, and gold chips with immobilized initiators were placed in a sealed reaction tube and deoxygenated via vacuum and nitrogen purging. CBAA monomer was deoxygenated in a separate sealed tube, and then dissolved in a deoxygenated solution of methanol and pure water in a 10:1 volume ratio. The monomer solution was transferred to the reaction tube using a syringe under nitrogen protection. In a shaker at 120 RPM and 25° C., pCBAA was allowed to react for 3 hr. After polymerization, chips were removed, rinsed with pure water and PBS, and stored overnight in PBS. Chips were rinsed with Milli-Q water and dried with filtered air just prior to any experiments. Dry film thickness was measured with an ellipsometer (J. A. Woollam, Alpha-SE), and chips with thicknesses of 20-30 nm were used for SPR measurements.

Preparation of mixed-charge peptide SAMs. Peptides CGG(KE)₂₀GPQG (referred to as poly(KE)₁, (SEQ ID NO: 2)) and CGG(KE)₂₀IWGQ (referred to as poly(KE)₂, (SEQ ID NO: 3)) were purchased from Synthetic Biomolecules (San Diego, Calif.) at a purity of >95%. Gold-coated chips were cleaned by rinsing with Millipore water and ethanol (Decon Laboratories, Inc., King of Prussia, Pa.) and then drying with filtered air. They were placed in the UV cleaner for 20 min. Cleaned gold chips were incubated in a phosphate buffered saline (PBS) solution (pH 7.4 and ionic strength 150 mM, Sigma-Aldrich, St. Louis, Mo.) containing 0.14 mM peptide for 24 hr. Once removed, the gold chips were rinsed with Millipore water and dried by filtered air.

Preparation of cell lysates. HSPCs were pelleted (1,000 rpm, 4° C.) and lysed into RIPA buffer (Sigma) which enables efficient cell lysis and protein solubilization while avoiding protein degradation and interference with the proteins' immunoreactivity and biological activity. The protein content of purified cell lysates was determined via BCA assay.

Measurements of protein adsorption. For protein adsorption tests, 1% HSPC lysates and 5GF-SFEM II were used for the protein absorption assay. This study used a custom-built surface plasmon resonance (SPR) sensor from the Institute of Photonics and Electronics, Academy Sciences (Prague, Czech Republic). A prepared chip was attached to the base of the prism and optical contact was established using refractive index matching fluid (Cargille). A quadruple-channel flow cell with four independent parallel flow channels was used to contain liquid samples during experiments. A peristaltic pump (Ismatec) was utilized to deliver liquid samples to the four channels of the flow cell. A stable baseline was first established with PBS, then protein solutions were delivered to the surface at a flow rate of 0.050 mL/min for 30 min, and PBS flowed again for 10 min before determining final wavelength shifts. A surface-sensitive SPR detector was used to monitor surface interactions in real time, and wavelength shift was used as an indication of changes on the surface.

Metabolite analysis. Cell-hydrogel constructs were washed in PBS and crushed with a tissue grinder before metabolites were extracted using an extraction solvent (1:3:1 Chloroform:Methanol:Water), and placed on a rotary shaker for 1 hr at 4° C. The solution was then centrifuged for 3 min at 13,000 g at 4° C. after which the supernatant was collected and dried at 30° C. for 2 hr in a SpeedVac. The samples were analyzed using a LC-MS QqQ method system as described below.

Liquid chromatography conditions. The LC system was composed of two Agilent 1260 binary pumps, an Agilent 1260 auto-sampler, and an Agilent 1290 column compartment containing a column-switching valve (Agilent Technologies, Santa Clara, Calif.). Each sample was injected twice: 10 μL for analysis using negative ionization mode, and 2 μL for analysis using positive ionization mode. Both chromatographic separations were performed in hydrophilic interaction chromatography (HILIC) mode on two SeQuant ZIC-c HILIC columns (150×2.1 mm, 3.0 μm particle size, Merck KGaA, Darmstadt, Germany) connected in parallel. This setup allows one column to be performing separation while the other column is being reconditioned to prepare for the next injection. The flow rate was 0.300 mL/min, auto-sampler temperature was kept at 4° C., the column compartment was set at 40° C., and total separation time for both ionization modes was 20 min. The mobile phase was composed of solvents A (5 mM ammonium acetate in 90% H₂O/10% acetonitrile+0.2% acetic acid) and B (5 mM ammonium acetate in 90% acetonitrile/10% H₂O+0.2% acetic acid). The gradient conditions for both separations were identical and are shown below.

| Time Segment, min. | Solvent A, % | Solvent B, % |
|---|---|---|
| 0-2 | 25 | 75 |
| 2-5 | 25 to 70 | 75 to 30 |
| 5-9 | 70 | 30 |
| 9-11 | 70 to 25 | 30 to 75 |
| 11-20 | 25 | 75 |

The metabolite identities were confirmed by spiking the sample used for method development with mixtures of standard compounds (each mixture contained five standard metabolites). All the samples were analyzed over a 12-day period and the retention times (RT) did not undergo any significant shift (each peak was within 6 seconds throughout 12 days of analysis), which proved the robustness of the HILIC method.

Mass spectrometry conditions. After the chromatographic separation, MS ionization and data acquisition were performed using an QTRAP® 5500 (AB Sciex, Toronto, ON, Canada) mass spectrometer equipped with an electrospray ionization (ESI) source. The instrument was controlled by Analyst 1.5 software (AB Sciex, Toronto, ON, Canada). Targeted data acquisition was performed in multiple-reaction-monitoring (MRM) mode. Ninety-nine and 59 MRM transitions were monitored in negative and positive mode, respectively (158 transitions in total). The source and collision gas was N₂ (99.999% purity). The ion source conditions in negative/positive mode were: curtain gas (CUR)=25 psi, collision gas (CAD)=high, ion spray voltage (IS)=−3.8/3.8 KV, temperature (TEM)=500° C., ion source gas 1 (GS1)=50 psi, and ion source gas 2 (GS2)=40 psi. The extracted MRM peaks were integrated using MultiQuant 2.1 software (AB Sciex, Toronto, ON, Canada).

Data analysis, model development and cross validation. A custom metabolite database incorporating HMDB was used to identify compounds. After exporting from MultiQuant software, spectral data were normalized using average values from the data of quality control (QC) injections (at least five in each batch, 33 QC samples in total). Means and standard errors of the mean were generated for all groups of picked peaks.

Cell sources. Human umbilical cord blood samples were obtained for research purposes from normal deliveries under Swedish Medical Center's Institutional Review Board (Seattle) approval and after consent was obtained. Unit processing and CD34+ cell selection were performed as described previously (Delaney et al., Nat Med, 16(2):232 (2010)). Bone marrow stem cells were purchased from Cooperative Centers for Excellence in Hematology (CCEH) at the Fred Hutchinson Cancer Research Center.

CD34+ HSPCs expansion and harvest in ZTG and HYSTEM® hydrogel. HSPC-hydrogel constructs were prepared between Rain-X-treated glass slides spaced at a known distance (150 μm), and reacted for 30 min at 37° C. HSPCs were encapsulated in ZTG at various seeding densities. To encapsulate HSPCs, cells were suspended in HSPC expansion media including StemSpan SFEM II (StemCell Technologies) supplemented with human 50 ng/mL stem cell factor (SCF), 50 ng/mL FMS-like tyrosine kinase 3 ligand (FLT3), 50 ng/mL thrombopoietin (TPO), 50 ng/mL interleukin-6 (IL-6) and 10 ng/mL interleukin-3 (IL-3) (Invitrogen). Media aliquots containing zwitterionic polymer and peptide (JPT Innovative peptide solutions), with total polymer concentration of 4% (w/v) (polymer/peptide molar ratio: 1:2) to allow gelation. The slides were separated, and the cell-encapsulating hydrogels were equilibrated in media. During the first 5-hour equilibration, the media was refreshed every hour, and then refreshed every day. The resulting hydrogels embedded with cells were incubated at 37° C. in 5% CO₂ and 100% relative humidity to promote cell expansion. As control, selected CD34+ cells were cultured directly in tissue culture polystyrene flasks or in 2.5 μg/mL Delta1 ext-IgG coated flasks (Delaney et al, 2010, supra). Culture media and supplements were the same as above. HYSTEM® hydrogels (Sigma) were prepared and dissolved according to the manufacturers instruction. For the no-growth-factors experiment, CD34+ cells were cultured in either ZTG or control condition using SFEM II media without other supplements. Metalloproteinase PBS solution (1 μg/mL; 37° C., 1 hr incubation) was used to fully dissolve the cell-hydrogel construct at certain time point to harvest expanded cells. During the hydrogel culture, oxygen sensor patches were used (Presens, Regensburg, Germany) to measure the dissolved oxygen (DO) level in hydrogels (Shen et al., Biomaterials Science, 2:655 (2014).

Flow cytometric analysis. Cell phenotypes of fresh and expanded cells were measured using a combination of the following antibodies and fluorophores: Primitive panel: APC-anti-huCD34, PECY7-huCD45RA, AF700-huCD38, PE-huCD90, FITC-huCD49f; Differentiation panel: AF700-huCD34, PE-huCD7, PeCy7-huCD56, APC-Cy7-huCD14, FITC-huCD15, and APC-huCD19 antibodies. ROS, mitochondrial mass, and mitochondrial membrane potential panel: DCF-DA (20-70-dichlorofluorescein diacetate; Molecular Probes) for ROS level, DHE (dihydroethidium, Molecular Probes) for intracellular O₂⁻, MitoTracker Green FM (Molecular Probes) for mitochondrial mass and MitoTrackerRedFM (Molecular Probes) for mitochondrial membrane potential. Signaling pathway panel: APC-phospho p38MAPK (eBioscience), PE-phospho mTOR (eBioscience) and AF48813-Catenin (eBioscience). Cell events were collected with an LSR II Flow Cytometer (BD Biosciences), and flow data was analyzed using FlowJo software (TreeStar, Ashland, Oreg.).

Assessment of cell morphology. Cell morphology was assessed using slides prepared by Cytospin using a cytocentrifuge (Cytospin 2, Shandon Scientific) at 500 rpm for 3 min followed by Wright-Giemsa staining. The bright field slides were scanned with Aperio Scanscope AT. The images were recorded and analyzed using Aperio ImageScope v12.2.1.5005. Briefly, the diameters of 50 randomly selected cells from each group were averaged and then compared between the groups.

In vitro colony-forming unit assays. Frequencies of colony-forming cells were estimated by plating 200 or 500 fresh or expanded CD34+ cells in methylcellulose-containing media (MethoCult H4434, StemCell Technologies) supplemented with TPO, and FLT3, each at a final concentration of 50 ng/mL. After 14 days in culture, plates were visually scored for CFU-multilineage colonies.

In vivo repopulation studies. All experiments with animals were conducted under protocols approved for use by the Fred Hutchinson Cancer Research Center Institutional Animal Care and Use Committee (IACUC). NOD/LtSz-scid IL2rg^{(-/-)} (NSG) mice were obtained from the Jackson Laboratory (Bar Harbor, Me.) and bred in the animal care facility at the Fred Hutchinson Cancer Research Center. Fresh cord blood CD34+ cells or their progeny present in different cultures were injected into sub-lethally irradiated (275 cGy) 5-10 weeks old NSG mice one day prior to infusion via tail vein. Their repopulating ability was assessed at 4 weeks and 12-14 weeks after transplant with marrow removed from the knee joint of anesthetized mice. At 24-30 weeks after transplant, the mice were sacrificed and both femurs and tibias were assessed for the numbers and types of human cells. For secondary transplants, 50% of the bone marrow isolated from each recipient mouse was transplanted into one secondary sub-lethally irradiated NSG mouse. Human cell engraftment was monitored by flow cytometric analysis of bone marrow cells obtained at week-4 using PE.Cy5-anti-human CD45 and APC.Cy7-anti-mouse CD45.1 antibodies. The subsets of human CD45⁺ cells were further determined using PE.Cy7-anti-huCD33, APC-anti-huCD19, PE-anti-huCD56, APC, Cy7-anti-huCD3, PE-anti-huCD41, FITC-anti-huCD235a, AlexaFluor700-anti-huCD34 or APC-anti-huCD34 and AlexaFluor700-anti-huCD38 antibodies were used. The frequency of SCID-repopulating cell (SRC) was determined by LDA. HSPCs either from fresh isolated cord blood CD34+ cells or from cultured cells were diluted serially to the desired cell doses. The frequency of SRC were calculated using ELDA software provided by the Walter and Eliza Hall Institute (Hu & Smyth, J Immunol Methods, 347:70 (2009)).

Cell cycle analysis. For the combinatory staining of surface markers and cell cycle, HSPC were stained with APC-anti-CD34 as described above. After a washing step, the cells were fixed and permeabilized with 70% ethanol, then intracellularly stained with FITC conjugated Ki-67. After washing, cells were stained with Hoechst33342 for 10 min and washed before resuspension in 100 μL PBS. Cells were subsequently analyzed using the LSRII flow cytometer (BD Biosciences).

RNA extraction. Total RNA was isolated using RNeasy micro Kit (Qiagen, Hilden, Germany) per the manufacture's recommendations and treated with RNAse-free DNAse (Qiagen, Hilden, Germany) to eliminate any DNA contaminant. The RNA concentration was measured using a Nanodrop 2000 spectrophotometer (Thermo Fisher Scientific, Waltham, Mass.).

RNA QC. Total RNA integrity was checked using an Agilent 2200 TapeStation (Agilent Technologies, Inc., Santa Clara, Calif.) and quantified using a Trinean DropSense96 spectrophotometer (Caliper Life Sciences, Hopkinton, Mass.).

RNA-seq expression analysis. RNA-seq libraries were prepared from total RNA using the SMARTer Stranded Total RNA-Seq Kit—Pico Input Mammalian (Clontech Laboratories, Inc., Mountain View, Calif., USA). Library size distributions were validated using an Agilent 2200 TapeStation (Agilent Technologies, Santa Clara, Calif., USA). Additional library QC, blending of pooled indexed libraries, and cluster optimization were performed using a QUBIT®2.0 Fluorometer (Life Technologies-Invitrogen, Carlsbad, Calif., USA). RNA-seq libraries were pooled (6-plex) and clustered onto a flow cell lane. Sequencing was performed using an Illumina HiSeq 2500 in “rapid run” mode employing a paired-end, 50 base read length (PE50) sequencing strategy. Image analysis and base calling were performed using Illumina's Real Time Analysis v1.18 software, followed by ‘demultiplexing’ of indexed reads and generation of FASTQ files, using Illumina's bcl2fastq Conversion Software v1.8.4.

RNA-seq data analysis. Reads of low quality were filtered prior to alignment to the reference genome (UCSC hg38 assembly) using TopHat v2.0.14 (Trapnell, et al., Bioinformatics, 25:1105 (2009)). Counts were generated from TopHat alignments for each gene using the Python package HTSeq v0.6.1 (Anders, et al., Bioinformatics, btu638 (2014)). Non-protein-coding genes were omitted prior to employing the Bioconductor package HTS Filter (Rau, et al., Bioinformatics, 29:2146 (2013)) to discard genes with low counts across conditions. Samples were paired by culture and differentially expressed genes were detected using the Bioconductor package edgeR v3.12.0 (Robinson, et al., Bioinformatics, 26:139 (2010)). A false discovery rate (FDR) method was employed to correct for multiple testing (Reiner, et al., Bioinformatics, 19:368 (2003)). Differential expression was defined as |log₂ (ratio)|≥1 (±2-fold) with the FDR set to 5%. Overrepresented GO Biological Process terms for genes which were either up or down-regulated were identified using the Bioconductor package GOseq (Young, et al., Genome Biology, 11(2):R14 (2010)), and a FDR method was employed to correct for multiple testing. GO terms were determined to be significant at an FDR of 5%, and were summarized and clustered based on semantic similarity measures using the online tool REVIGO (Supek, et al., PloS One, 6(7):e21800 (2011)). A core analysis was performed using the Ingenuity Knowledge Base (Genes Only) as a reference set and employing default parameters. The analysis was used to determine enriched canonical pathways in the dataset. Enrichment was scored using the Fisher's Exact Test.

Accession codes. Gene Expression Omnibus: GSE85800.

As will be understood by one of ordinary skill in the art, each embodiment disclosed herein can comprise, consist essentially of or consist of its particular stated element, step, ingredient or component. “Includes” or “including” means “comprises, consists essentially of or consists of.” The transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would result in substantial increase in differentiation during ZTG expansion such that differentiation was not statistically significantly different from culturing using DXIₒₚₜ as a relevant control condition.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the present disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the disclosure are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Particular embodiments of this disclosure are described herein, including the best mode known to the inventors for carrying out the disclosure. Of course, variations on these described embodiments will become apparent to those of ordinary skill in the art upon reading the foregoing description. The inventors expect skilled artisans to employ such variations as appropriate, and the inventors intend for the disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

Furthermore, numerous references have been made to books, journal articles, treatises, patents, printed publications, etc. (collectively “references”) throughout this specification. Each of the above-cited references is individually incorporated by reference herein for its cited teachings.

In closing, it is to be understood that the embodiments of the disclosure are illustrative of the principles of the present disclosure. Other modifications that may be employed are within the scope of the disclosure. Thus, by way of example, but not of limitation, alternative configurations of the present disclosure may be utilized in accordance with the teachings herein. Accordingly, the present disclosure is not limited to that precisely as shown and described.

The particulars shown herein are by way of example and for purposes of illustrative discussion of the preferred embodiments of the present disclosure only and are presented in the cause of providing what is believed to be the most useful and readily understood description of the principles and conceptual aspects of various embodiments of the disclosure. In this regard, no attempt is made to show structural details of the disclosure in more detail than is necessary for the fundamental understanding of the disclosure, the description taken with the drawings and/or examples making apparent to those skilled in the art how the several forms of the disclosure may be embodied in practice.

Definitions and explanations used in the present disclosure are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Ed. Anthony Smith, Oxford University Press, Oxford, 2004).

What is claimed is:

1. A method of expanding hematopoietic stem cells (HSC), comprising
culturing a CD34+ hematopoietic cell population within a zwitterionic hydrogel (ZTG) in expansion medium comprising human stem cell factor (SCF), FMS-like tyrosine kinase 3 ligand (FLT3), thrombopoietin (TPO), interleukin-6 (IL-6), and interleukin-3 (IL-3), for a period sufficient to provide a ZTG-expanded HSC population comprising at least a 10-fold increase in the total number of HSC as compared to the starting CD34+ hematopoietic cell population.

2. A method of claim 1, wherein following the expansion the ZTG-expanded HSC population has a lower metabolic rate as compared to a control CD34+ hematopoietic cell population expanded in a relevant control condition comprising expansion within a hydrophobic polystyrene flask or with a Notch agonist substrate.

3. A method of claim 2, wherein the lower metabolic rate is demonstrated through a reduction in one or more of (i) glucose consumption; (ii) lactate secretion; and (iii) amino acid metabolism.

4. A method of claim 2, wherein the lower metabolic rate is demonstrated through a reduction in mitochondrial mass and/or mitochondrial membrane potential.

5. A method of claim 1, wherein following the expansion the ZTG-expanded HSC population has a higher proportion of HSC in a quiescent state as compared to a control CD34+ hematopoietic cell population expanded in a relevant control condition comprising expansion within a hydrophobic polystyrene flask or with a Notch agonist substrate.

6. A method of claim 5, wherein the higher proportion of HSC in a quiescent state is demonstrated through a reduced proportion of G₁ cells and an increased proportion of G₀ cells.

7. A method of claim 1, wherein the CD34+ hematopoietic cell population expanding within the ZTG has decreased production of reactive oxygen species (ROS) as compared to a control CD34+ hematopoietic cell population expanding within a relevant control condition comprising expansion within a hydrophobic polystyrene flask or with a Notch agonist substrate.

8. A method of claim 1, further comprising isolating the CD34+ hematopoietic cell population for expansion from umbilical cord blood, peripheral blood, mobilized peripheral blood, bone marrow, or embryonic or induced pluripotent stem cells.

9. A method of claim 1, wherein the HSC are human HSC.

10. A method of claim 1, wherein the HSC are enriched for CD34+ HSC prior to the ZTG-expansion.

11. A method of claim 1, wherein the ZTG comprises a zwitterionic polymer, polyethylene glycol, or a saccharide.

12. A method of claim 11, wherein the ZTG comprises an acrylamide polymer crosslinked by a biodegradable peptide.

13. A method of claim 12, wherein the biodegradable peptide comprises a poly(EK) crosslinker.

14. A method of claim 13, wherein the poly(EK) crosslinker comprises a bis(azide) di-functionalized polypeptide.

15. A method of claim 12, wherein the ZTG comprises a zwitterionic four-arm poly(carboxybetaine acrylamide) tetracyclooctyne polymer crosslinked by a biodegradable azide-GG-(KE)₂₀-GPQGIWGQ-(KE)₂₀GG-azide (SEQ ID NO: 1) peptide.

16. A method of claim 1, further comprising crosslinking the ZTG in a suspension comprising the ZTG, a crosslinker, and the CD34+ hematopoietic cell population.

17. A method of claim 1, further comprising forming the ZTG via a copper-free, strain-promoted azide-alkyne cycloaddition reaction between terminal difluorinated cyclooctyne and azide moieties.

18. A method of claim 1, wherein the culturing is conducted for at least 10 days.

19. A method of claim 1, further comprising recovering HSC from the ZTG-expanded HSC population for a research or clinical use or for further expansion.

20. A method of claim 19, wherein the recovering comprises degrading the ZTG with a metalloproteinase.

21. A method of claim 1, wherein the ZTG-expanded HSC population comprises at least a 10-fold increase in the total number of HSC having a CD34+phenotype as compared to the starting CD34+ hematopoietic cell population.

22. A method of claim 1, wherein the ZTG-expanded HSC population comprises at least a 10-fold increase in the total number of HSC having a CD34+, CD38−, CD45RA−, CD49f+, CD90+ phenotype as compared to the starting CD34+ hematopoietic cell population.

23. A method of claim 1, wherein at least 50% of the HSC in the ZTG-expanded HSC population are CD34+ and Lin− or at least 50% of the HSC in the ZTG-HSC population are CD34+ and CD45RA−.

24. A method of claim 23, wherein at least 90% of the HSC in the ZTG-expanded HSC population are CD34+ and Lin− or at least 70% of the HSC in the ZTG-HSC population are CD34+ and CD45RA−.

25. A method of claim 1, further comprising genetically modifying cells within the CD34+ hematopoietic cell population.

26. A method of claim 1, further comprising genetically modifying cells within the ZTG-expanded HSC population.

27. A method of expanding hematopoietic stem cells (HSC) comprising:
incorporating a CD34+ hematopoietic cell population into a zwitterionic hydrogel (ZTG) and culturing the incorporated cells under conditions that result in a ZTG-expanded HSC population,
wherein the ZTG is crosslinked by a peptide that is degraded by a product excreted by one or more cells within the expanding cell population.

28. A method of claim 27, wherein the ZTG-expanded HSC population has an increased proportion of HSC versus partially or fully differentiated cells as compared to a control-expanded CD34+ hematopoietic cell population expanded in a relevant control condition comprising expansion within a hydrophobic polystyrene flask or with a Notch agonist substrate.

29. A method of claim 28, wherein the increased proportion of HSC versus partially or fully differentiated cells is demonstrated through a difference in (i) a fold increase in HSC having a CD34+, CD38−, CD45RA−, CD49f+, CD90+ phenotype in the ZTG-expanded HSC population; (ii) a percentage increase of HSC that are CD34+ and Lin− in the ZTG-expanded HSC population; and/or (iii) a percentage increase in HSC that are CD34+ and CD45RA− in the ZTG-expanded HSC population.

30. A method of claim 27, wherein the ZTG-expanded HSC population has a reduced metabolic rate following expansion as compared to a control-expanded CD34+ hematopoietic cell population expanded in a relevant control condition comprising expansion within a hydrophobic polystyrene flask or with a Notch agonist substrate.

31. A method of claim 30, wherein the reduced metabolic rate is demonstrated through a reduction in one or more of (i) glucose consumption; (ii) lactate secretion; and/or (iii) amino acid metabolism.

32. A method of claim 30, wherein the reduced metabolic activity is demonstrated through a reduction in mitochondrial mass and/or mitochondrial membrane potential.

33. A method of claim 27, wherein following the expansion the ZTG-expanded HSC population has a higher proportion of HSC in a quiescent state as compared to a control CD34+ hematopoietic cell population expanded in a relevant control condition comprising expansion within a hydrophobic polystyrene flask or with a Notch agonist substrate.

34. A method of claim 33, wherein the higher proportion of HSC in a quiescent state is demonstrated through a reduced proportion of G1 cells and an increased proportion of G0 cells.

35. A method of claim 27, wherein the CD34+ hematopoietic cell population expanding within the ZTG has decreased production of ROS as compared to a control CD34+ hematopoietic cell population expanding within a relevant control condition comprising expansion within a hydrophobic polystyrene flask or with a Notch agonist substrate.

36. A method of claim 27, wherein the ZTG comprises a zwitterionic polymer, polyethylene glycol, or a saccharide.

37. A method of claim 27, wherein the ZTG comprises a zwitterionic polymer comprising a four-arm poly(carboxybetaine acrylamide) tetracyclooctyne.

38. A method of claim 27, wherein the peptide comprises a poly(EK) crosslinker.

39. A method of claim 38, wherein the poly(EK) crosslinker comprises a bis(azide) di-functionalized polypeptide.

40. A method of claim 39, wherein the bis(azide) di-functionalized polypeptide comprises Azide-GG-(KE)₂₀-GPQGIWGQ-(KE)₂₀GG-Azide (SEQ ID NO: 1).

41. A method of claim 27, wherein the product is metalloproteinase.

42. A method of claim 27, further comprising isolating the CD34+ hematopoietic cell population for expansion from umbilical cord blood, peripheral blood, mobilized peripheral blood, bone marrow, or embryonic or induced pluripotent stem cells.

43. A method of claim 27, wherein the CD34+ hematopoietic cell population for expansion comprises human HSC.

44. A method of claim 27, wherein the CD34+ hematopoietic cell population for expansion are isolated from umbilical cord blood.

45. A method of claim 27, further comprising forming the ZTG via a copper-free, strain-promoted azide-alkyne cycloaddition reaction between terminal difluorinated cyclooctyne and azide moieties.

46. A method of claim 27, further comprising crosslinking the ZTG in a suspension comprising a zwitterionic polymer, the peptide, and the CD34+ hematopoietic cell population for expansion.

47. A method of claim 46, further comprising suspending the CD34+ hematopoietic cell population for expansion in expansion media comprising SCF, FLT3, TPO, IL-6, and/or IL-3.

48. A method of claim 46, further comprising suspending the CD34+ hematopoietic cell population for expansion in expansion media comprising SCF, FLT3, TPO, IL-6, and IL-3.

49. A method of claim 27, wherein culturing is continued for at least 10 days.

50. A method of claim 27, further comprising further degrading the ZTG; recovering HSC from the ZTG-expanded HSC population; and encapsulating the recovered HSC within a second hydrogel comprising a zwitterionic polymer.

51. A method of claim 27, further comprising further degrading the ZTG after the culturing, such that the ZTG-expanded HSC population is no longer incorporated within the ZTG; and harvesting HSC from the ZTG-expanded HSC population for a use.

52. A method of claim 51, wherein the degrading utilizes metalloproteinase.

53. A method of producing an expanded hematopoietic stem cells (HSC) population with reduced metabolic activity comprising: expanding HSC within a hydrogel comprising a zwitterionic polymer crosslinked with a biodegradable Azide-GG-(KE)₂₀-GPQGIWGQ-(KE)₂₀GG-Azide (SEQ ID NO: 1) peptide, wherein following the expansion, the hydrogel-expanded HSC population has reduced metabolic activity as demonstrated through one or more parameters selected from (i) reduced glucose consumption, (i) reduced lactate secretion, and (iii) reduced amino acid metabolism, as compared to HSC expanded in a relevant control condition, wherein the relevant control condition comprises expansion using a hydrophobic polystyrene flask or with a Notch agonist substrate.

54. A method of claim 53, wherein the hydrogel-expanded HSC population has an increased proportion of HSC versus partially or fully differentiated cells as compared to the control-expanded HSC.

55. A method of claim 53, wherein the increased proportion of HSC is demonstrated through a difference in (i) a fold increase in HSC having a CD34+, CD38−, CD45RA−, CD49f+, CD90+ phenotype in the hydrogel-expanded HSC population; (ii) a percentage increase of HSC that are CD34+ and Lin− in the hydrogel-expanded HSC population; and/or (iii) a percentage increase in HSC that are CD34+ and CD45RA− in the hydrogel-expanded HSC population.

56. A method of claim 53, wherein the reduced metabolic activity is demonstrated through a reduction in mitochondrial mass and/or mitochondrial membrane potential.

57. A method of claim 53, wherein following the expansion the ZTG-expanded HSC population has a higher proportion of HSC in a quiescent state.

58. A method of claim 57, wherein the higher proportion of HSC in a quiescent state is demonstrated through a reduced proportion of G1 cells and an increased proportion of G0 cells.

59. A method of claim 53, wherein the zwitterionic polymer comprising a four-arm poly(carboxybetaine acrylamide) tetracyclooctyne.

60. A method of claim 53, further comprising isolating the HSC for expansion from umbilical cord blood, bone marrow, or embryonic or induced pluripotent stem cells.

61. A method of claim 53, wherein the HSC are human HSC.

62. A method of claim 53, further comprising forming the hydrogel via a copper-free, strain-promoted azide-alkyne cycloaddition reaction between terminal difluorinated cyclooctyne and azide moieties.

63. A method of claim 53, further comprising suspending the HSC for expansion in expansion media comprising SCF, FLT3, TPO, IL-6, and/or IL-3.

64. A method of claim 53, further comprising suspending the HSC for expansion in expansion media comprising SCF, FLT3, TPO, IL-6, and IL-3.

65. A method of claim 53, wherein the expanding occurs over a period of at least 10 days.

66. A method of claim 53, further comprising degrading the hydrogel; recovering hydrogel-expanded HSC from the hydrogel-expanded HSC population; and encapsulating the recovered hydrogel-expanded HSC within a second hydrogel comprising a zwitterionic polymer.

67. A method of claim 53, further comprising degrading the hydrogel such that the hydrogel-expanded HSC population is not incorporated within the hydrogel; and harvesting hydrogel-expanded HSC from the hydrogel-expanded HSC population for a use.

68. A method of claim 67, wherein the degrading utilizes metalloproteinase.

69. A method of claim 68, wherein the use is for long-term hematopoietic reconstitution in a subject in need thereof.

70. A method of repopulating an immune system in a subject in need thereof including: administering a therapeutically effective amount of a composition comprising a zwitterionic hydrogel (ZTG)-expanded hematopoietic stem cells (HSC) population to the subject, thereby repopulating the immune system of the subject in need thereof.

71. A method of claim 70, wherein the repopulating provides long-term hematopoietic reconstitution.

72. A method of claim 70, wherein the subject is a human subject.

73. A method of claim 70, wherein at least a portion of cells within the ZTG-expanded HSC population are genetically modified.

74. A method of claim 73, wherein the genetic modification results in expression of a therapeutic gene or gene product.

75. A method of claim 70, wherein the therapeutically effective amount provides a prophylactic treatment and/or a therapeutic treatment.

76. A method of claim 70, wherein the subject is in need thereof due to exposure to one or more of an alkylating agent, Ara-C, azathioprine, carboplatin, cisplatin, chlorambucil, clofarabine, cyclophosphamide, ifosfamide, mechlorethamine, mercaptopurine, oxaliplatin, taxanes, vincristine, vinblastine, vinorelbine, or vindesine.

77. A method of claim 70, wherein the subject is in need thereof due to exposure to a myeloablative regimen for hematopoietic cell transplantation.

78. A method of claim 70, wherein the subject is in need thereof due to exposure to acute ionizing radiation or exposure to drugs that cause bone marrow suppression or hematopoietic deficiencies including at least one of an antibiotic, penicillin, ganciclovir, daunomycin, a sulfa drug, a phenothiazine, a tranquilizer, meprobamate, an analgesic, aminopyrine, dipyrone, an anticonvulsant, phenytoin, carbamazepine, an antithyroid, propylthiouracil, methimazole, or a diuretic.

79. A method of claim 70, wherein the subject is in need thereof due to a viral infection, a microbial infection, or a parasitic infection as a result of treatment for renal disease or renal failure.

80. A method of claim 70, wherein the subject is in need thereof due to an immunodeficiency in at least one of T or B lymphocytes, or rheumatoid arthritis.

81. A method of claim 70, wherein the subject is in need thereof due to at least one of aplastic anemia, Chediak-Higashi syndrome, systemic lupus erythematosus (SLE), leukemia, myelodysplastic syndrome, myelofibrosis, or thrombocytopenia.

82. A method of claim 70, wherein the subject is in need thereof due to trauma-related blood loss.

83. A method of claim 70, wherein the subject receives at least a portion of the expanded HSC population before, at the same time, or after chemotherapy, radiation therapy, or a bone marrow transplant.

84. A method of claim 70, wherein the subject is in need thereof due to an immunodeficiency, a pancytopenia, a neutropenia, or a leukopenia.

85. A method of claim 73, wherein the genetic modification leads to expression of a therapeutic gene or gene product including soluble CD40; CTLA; Fas L; antibodies to CD4, CD5, CD7, CD52; antibodies to IL1, IL2, IL6; an antibody to TCR specifically present on autoreactive T cells; IL4; IL10; IL12; IL13; IL1Ra, sIL1RI, sIL1RII; sTNFRI; sTNFRII; antibodies to TNF; P53, PTPN22, and DRB1*1501/DQB1*0602; globin family genes; WAS; phox; FANC family genes; dystrophin; pyruvate kinase; CLN3; ABCD1; arylsulfatase A; SFTPB; SFTPC; NLX2.1; ABCA3; GATA1; ribosomal protein genes; TERT; TERC; DKC1; TINF2; CFTR; LRRK2; PARK2; PARK7; PINK1; SNCA; PSEN1; PSEN2; APP; SOD1; TDP43; FUS; ubiquilin 2; C9ORF72, IDUA or iduronidase, IDS, GNS, HGSNAT, SGSH, NAGLU, GUSB, GALNS, GLB1, ARSB, and HYAL1, 101F6, 123F2 (RASSF1), 53BP2, abl, ABLI, ADP, aFGF, APC, ApoAl, ApoAlV, ApoE, ATM, BAI-1, BDNF, Beta*(BLU), bFGF, BLC1, BLC6, BRCA1, BRCA2, CBFA1, CBL, C-CAM, CFTR, CNTF, COX-1, CSFIR, CTS-1, cytosine deaminase, DBCCR-1, DCC, Dp, DPC-4, E1A, E2F, EBRB2, erb, ERBA, ERBB, ETS1, ETS2, ETV6, Fab, FCC, FGF, FGR, FHIT, fms, FOX, FUS 1, FUS1, FYN, G-CSF, GDAIF, Gene 21 (NPRL2), Gene 26 (CACNA2D2), GM-CSF, GMF, gsp, HCR, HIC-1, HRAS, hst, IGF, IL-1, IL-2, IL-3, IL-4, IL-5, IL-6, IL-7, IL-8, IL-9, IL-10, IL-11 IL-12, ING1, interferon α, interferon β, interferon γ, IRF-1, JUN, KRAS, LCK, LUCA-1 (HYAL1), LUCA-2 (HYAL2), LYN, MADH4, MADR2, MCC, mda7, MDM2, MEN-I, MEN-II, MLL, MMAC1, MYB, MYC, MYCL1, MYCN, neu, NF-1, NF-2, NGF, NOEY1, NOEY2, NRAS, NT3, NT5, OVCA1, p16, p21, p27, p53, p57, p73, p300, PGS, PIM1, PL6, PML, PTEN, raf, Rap1A, ras, Rb, RB1, RET, rks-3, ScFv, scFV ras, SEM A3, SRC, TAL1, TCL3, TFPI, thrombospondin, thymidine kinase, TNF, TP53, trk, T-VEC, VEGF, VHL, VVT1, WT-1, YES, zac1, α2β1; ανβ3; ανβ5; ανβ63; BOB/GPR15; Bonzo/STRL-33/TYMSTR; CCR2; CCR3; CCR5; CCR8; CD4; CD46; CD55; CXCR4; aminopeptidase-N; HHV-7; ICAM; ICAM-1; PRR2/HveB; HveA; α-dystroglycan; LDLR/α2MR/LRP; PVR; PRR1/HveC; and laminin receptor.

86. A ZTG-expanded HSC population formed according to a method and including a feature of any of claims 1-68.

87. A ZTG-expanded HSC population of claim 86, wherein the feature includes a lower metabolic rate as compared to a control CD34+ hematopoietic cell population expanded in a relevant control condition comprising expansion within a hydrophobic polystyrene flask or with a Notch agonist substrate.

88. A ZTG-expanded HSC population of claim 87, wherein the lower metabolic rate is demonstrated through a reduction in one or more of (i) glucose consumption; (ii) lactate secretion; and (iii) amino acid metabolism.

89. A ZTG-expanded HSC population of claim 87, wherein the lower metabolic rate is demonstrated through a reduction in mitochondrial mass and/or mitochondrial membrane potential.

90. A ZTG-expanded HSC population of claim 86, wherein the feature includes a higher proportion of HSC in a quiescent state as compared to a control CD34+ hematopoietic cell population expanded in a relevant control condition comprising expansion within a hydrophobic polystyrene flask or with a Notch agonist substrate.

91. A ZTG-expanded HSC population of claim 90, wherein the higher proportion of HSC in a quiescent state is demonstrated through a reduced proportion of G₁ cells and an increased proportion of G₀ cells.

92. A ZTG-expanded HSC population of claim 86, wherein the ZTG-expanded HSC population comprises at least a 10-fold increase in the total number of HSC as compared to the starting CD34+ hematopoietic cell population from which it was formed.

93. A ZTG-expanded HSC population of claim 86, wherein the ZTG-expanded HSC population comprises at least a 10-fold increase in the total number of HSC having a CD34+, CD38−, CD45RA−, CD49f+, CD90+ phenotype as compared to the starting CD34+ hematopoietic cell population from which it was formed.

94. A ZTG-expanded HSC population of claim 86, wherein at least 50% of the HSC in the ZTG-expanded HSC population are CD34+ and Lin− or at least 50% of the HSC in the ZTG-HSC population are CD34+ and CD45RA−.

95. A ZTG-expanded HSC population of claim 86, wherein at least 90% of the HSC in the ZTG-expanded HSC population are CD34+ and Lin− or at least 70% of the HSC in the ZTG-HSC population are CD34+ and CD45RA−.

96. A ZTG-expanded HSC population of claim 86, wherein at least a portion of HSC within the ZTG-expanded HSC population are genetically modified.

97. A ZTG-expanded HSC population of claim 96, wherein the genetic modification includes insertion of an exogenous nucleotide sequence that leads to expression of a therapeutic gene or gene product comprising soluble CD40; CTLA; Fas L; antibodies to CD4, CD5, CD7, CD52; antibodies to IL1, IL2, IL6; an antibody to TCR specifically present on autoreactive T cells; IL4; IL10; IL12; IL13; IL1Ra, sIL1RI, sIL1RII; sTNFRI; sTNFRII; antibodies to TNF; P53, PTPN22, and DRB1*1501/DQB1*0602; globin family genes; WAS; phox; FANC family genes; dystrophin; pyruvate kinase; CLN3; ABCD1; arylsulfatase A; SFTPB; SFTPC; NLX2.1; ABCA3; GATA1; ribosomal protein genes; TERT; TERC; DKC1; TINF2; CFTR; LRRK2; PARK2; PARK7; PINK1; SNCA; PSEN1; PSEN2; APP; SOD1; TDP43; FUS; ubiquilin 2; C9ORF72, IDUA or iduronidase, IDS, GNS, HGSNAT, SGSH, NAGLU, GUSB, GALNS, GLB1, ARSB, and HYAL1, 101F6, 123F2 (RASSF1), 53BP2, abl, ABLI, ADP, aFGF, APC, ApoAl, ApoAlV, ApoE, ATM, BAI-1, BDNF, Beta*(BLU), bFGF, BLC1, BLC6, BRCA1, BRCA2, CBFA1, CBL, C-CAM, CFTR, CNTF, COX-1, CSFIR, CTS-1, cytosine deaminase, DBCCR-1, DCC, Dp, DPC-4, E1A, E2F, EBRB2, erb, ERBA, ERBB, ETS1, ETS2, ETV6, Fab, FCC, FGF, FGR, FHIT, fms, FOX, FUS 1, FUS1, FYN, G-CSF, GDAIF, Gene 21 (NPRL2), Gene 26 (CACNA2D2), GM-CSF, GMF, gsp, HCR, HIC-1, HRAS, hst, IGF, IL-1, IL-2, IL-3, IL-4, IL-5, IL-6, IL-7, IL-8, IL-9, IL-10, IL-11 IL-12, ING1, interferon α, interferon β, interferon γ, IRF-1, JUN, KRAS, LCK, LUCA-1 (HYAL1), LUCA-2 (HYAL2), LYN, MADH4, MADR2, MCC, mda7, MDM2, MEN-I, MEN-II, MLL, MMAC1, MYB, MYC, MYCL1, MYCN, neu, NF-1, NF-2, NGF, NOEY1, NOEY2, NRAS, NT3, NT5, OVCA1, p16, p21, p27, p53, p57, p73, p300, PGS, PIM1, PL6, PML, PTEN, raf, Rap1A, ras, Rb, RB1, RET, rks-3, ScFv, scFV ras, SEM A3, SRC, TAL1, TCL3, TFPI, thrombospondin, thymidine kinase, TNF, TP53, trk, T-VEC, VEGF, VHL, WT1, WT-1, YES, zac1, α2β1; ανβ3; ανβ5; ανβ63; BOB/GPR15; Bonzo/STRL-33/TYMSTR; CCR2; CCR3; CCR5; CCR8; CD4; CD46; CD55; CXCR4; aminopeptidase-N; HHV-7; ICAM; ICAM-1; PRR2/HveB; HveA; α-dystroglycan; LDLR/α2MR/LRP; PVR; PRR1/HveC; and laminin receptor.

98. A composition comprising a ZTG-expanded HSC population of claim 86.

99. A composition comprising a therapeutically effective amount of a ZTG-expanded HSC population of claim 86.
